(19) United States
(12) Patent Application Publication
(10) Pub. No.: US 2007/0207952 A1
(43) Pub. Date: Sep. 6, 2007

(54) Long Acting Biologically Active Conjugates

(75) Inventors: Abelardo Silva, Ellicott City, MD (US); John E. Erickson, Potomac, MD (US); Michael Eissenstat, Frederick, MD (US); Elena Afonina, Frederick, MD (US); Sergei Gulnik, Frederick, MD (US)

Correspondence Address: PROSKAUER ROSE LLP, 1001 PENNSYLVANIA AVE, N.W.,, SUITE 400 SOUTH, WASHINGTON, DC, 20004, US

(73) Assignee: SEQUOIA PHARMACEUTICALS, Gaithersburg, MD (US)

(21) Appl. No.: 10/550,715

(22) PCT Filed: Mar. 24, 2004
(86) PCT No.: PCT/US04/08847

§ 371 Date: Aug. 16, 2006

Related U.S. Application Data
(60) Provisional application No. 60/456,472, filed on Mar. 24, 2003.
(60) Provisional application No. 60/456,952, filed on Mar. 25, 2003.
(60) Provisional application No. 60/518,892, filed on Nov. 10, 2003.

(51) Int. Cl. A61K 38/16; A61K 38/10; C07K 14/47; C07K 7/08

(52) U.S. Cl. 514/12; 530/326; 530/324; 530/325; 514/13; 424/78.27

(57) ABSTRACT

The invention provides biologically active compounds that may be reacted with macromolecules, such as albumin, to form covalent linked complexes wherein the resulting complexes exhibit a desired biological activity in vivo. More specifically, the complexes are isolated complexes comprising a biologically active moiety covalently bound to a linking group and a protein. The complexes are prepared by conjugating a biologically active moiety, for example, a renin inhibitor or a viral fusion inhibitor peptide, with purified and isolated protein. The complexes have extended lifetimes in the bloodstream as compared to the unconjugated molecule, and exhibit biological activity for extended periods of time as compared to the unconjugated molecule. The invention also provides anti-viral compounds that are inhibitors of viral infection and/or exhibit anti-fusiogenic properties. In particular, this invention provides compounds having inhibiting activity against viruses such as human immunodeficiency virus (HIV), respiratory syncytial virus (RSV), human parainfluenza virus (HPV), measles virus (MeV), and simian immunodeficiency virus (SIV) and that have extended duration of action for the treatment of viral infections.

This application claims priority to provisional applications 60/518,892, filed Nov. 10, 2003, Ser. No. 60/456,472, filed Mar. 24, 2003, and Ser. No. 60/456,952, filed Mar. 25, 2003, the contents of each of which are hereby incorporated by reference in their entireties.

### FIELD OF INVENTION

The invention relates to biologically active compounds that may be used to react with proteins, such as albumin, to form covalent linked complexes wherein the resulting complexes exhibit a desired biological activity in vivo. More specifically, the complexes are isolated complexes comprising a biologically active moiety covalently bound to a linking group and a protein. In one embodiment, the protein is a blood protein such as albumin, or HSA. In another embodiment, the protein is recombinant HSA. The complexes are prepared by conjugating a biologically active moiety, for example, a renin inhibitor or a viral fusion inhibitor peptide, with purified and isolated protein. The complexes have extended lifetimes in the bloodstream as compared to the unconjugated molecule, and exhibit biological activity for extended periods of time as compared to the unconjugated molecule. Optionally, the compounds and complexes of the present invention are isolated and purified. The invention also provides methods for achieving a desired biological effect in vivo, comprising administering to the bloodstream of a mammalian host the novel isolated complexes of the present invention.

The invention also provides compounds, including nucleosides, nucleoside analogs, nucleotides, nucleotide analogs, polypeptides, polypeptide derivatives, peptidomimetic compounds and their bioconjugated forms that are inhibitors of virus infections. The invention also provides methods for administering bioconjugated forms of these inhibitors having an extended duration of action for the treatment of virus infections, including multidrug-resistant virus infections.

In particular, the invention provides compounds and their bioconjugated forms that inhibit human immunodeficiency viruses (HIV), and methods for administering bioconjugated forms of inhibitors that provide a prolonged duration of action for the treatment of HIV infections, including multidrug-resistant HIV infections (mdrHIV).

### BACKGROUND OF THE INVENTION

Certain active peptide and protein therapeutics useful for administration to a mammalian host exhibit poor pharmacokinetic profiles, and are often rapidly metabolized and cleared by the mammalian system before the peptide or protein can bind to a specific target. Typically, once administered, the biologically active agents are susceptible to enzyme degradation and clearance. As a consequence, certain active agents must be administered more frequently which result in undesired large fluctuations in the blood plasma levels of the agent can lead to a variety of adverse side reactions and/or diminished efficacy. For an effective therapeutic, the active agent must be able to be transported to the active site or must be administered directly to the target site without significant loss of biological activity.

The majority of drugs are administered orally. Typically, the administered dosage requires that the drug be administered repetitively to maintain a therapeutic level and the rapid decrease in blood levels over time often results in initial levels that exceeds the desired therapeutic levels. Various technological approaches have been designed to avoid these problems, including the administration of biologically active agents by mechanical systems such as pumps, controlled release or slow release tablets and capsules, depots and related technologies.

Therapeutic agents that are administered by injections encounter similar problems relating to their limited lifetime in vivo. Moreover, repetitive injections are inconvenient and highly undesirable. Therefore, there is a need for new methods that allow for ease of administration of biologically active agents into the bloodstream and that maintain effective levels of the therapeutic agents for an extended period of time in vivo.

HIV/AIDS

Acquired immune deficiency syndrome (AIDS) is a fatal disease caused by infection with HIV-1. By the end of 2002, over 42 million people will be infected with HIV-1 worldwide, and over 20 million individuals will have died of HIV/AIDS. Drug resistant strains of HV are prevalent on the patient population. Current estimates are that up to 50% of drug-treated HIV-infected patients harbor a drug-resistant strain of HIV. Transmission rates of dmg-resistant HIV are between 10-15% in the US alone. Estimates of reported cases in the very near future also continue to rise dramatically. Consequently, there is a great need to develop drugs and vaccines to combat AIDS.

The AIDS virus was first identified in 1983. It has been known by several names and acronyms. It was originally the third known T-lymphocyte virus (HTLV-III), and it has the capacity to replicate within cells of the immune system, causing profound cell destruction and impairment of immunity. The AIDS virus is a retrovirus, which is family of viruses that use reverse transcriptase during their replication. This particular retrovirus is also known as lymphadenopathy-associated virus (LAV), AIDS-related virus (ARV) and, most recently, as human immunodeficiency virus (HIV).

Viral Diversity:

HIV is a member of the lentivirus family of retroviruses, which includes simian immunodeficiency virus (SIV), and numerous other retroviruses that cause immunodeficency diseases in mammals. Two distinct types of HIV have been described to date, namely HIV-1 and HIV-2, although infection with HIV-1 is more common worldwide. The acronym HIV will be used herein to refer to all HIV-1 viruses generically, unless otherwise noted. HIV-1 is further divided into three groups: major (M), outlier (O), and new (N). Most HIV-1 isolates to date belong to one of ten distinct clades, or subtypes, of the M group. The M group subtypes are represented by the letters A-J. Subtype B is the most common in the US and Europe. However, subtype C accounts for almost 50% of HIV worldwide, and is most common in Africa. All subtypes are present in Africa, with non-C clades tending to be cluster in distinct geographical regions. Subtype identification is usually determined by sequencing of the env gene, and comparison of the gp41 sequences, which give a subtype “fingerprint”.

Viral Life Cycle:

HIV primarily infects CD4-bearing helper/inducer T-cells, and can also infect other cells that express the CD4 glycoprotein at the membrane surface. Recent evidence has shown that the co-localization of certain chemokine receptors at the cell surface is essential for efficient viral infection. HIV is cytopathic to CD4+ lymphocytes, and their numbers steadily decline of over a period of years, resulting in a severely compromised immune system. HIV infection can also result in neurological deterioration and dementia. Unless treated with effective chemotherapy, HIV infection is almost always fatal, and leads to death from opportunistic infections, cancer or neurodegenerative disease.

The HIV-1 genome contains at least nine different genes. Ihe largest genes are gag (coding for structural proteins), pol (coding for the viral enzymes—protease, reverse transcriptase and integrase) and env (coding for the envelope glycoproteins). Homologues of the gag, pol and env genes are found in all retroviruses

The gag and pol regions of the genome encode polycistronic messenger RNAs which are translated into large polyprotein precursors. The viral polyproteins are subsequently cleaved into mature structural proteins and enzymes by a viral-encoded protease that is, itself, a product of the pol gene. The two Env proteins, gp120 and gp41, are cleaved from a larger precursor (gp160) by a cellular enzyme.

Other HIV-1 gene products, e.g., Tat, Rev, Vpr, and Nef, intervene to regulate the virus life cycle. Nef also affects particle infectivity. The gene products Vif and Vpu function in virus infectivity and virus particle maturation, respectively. The viral genome is flanked at each end by long terminal repeat sequences (LTRs). The LTRs contain binding sites for cellular proteins that are able to activate transcription and are also under the control of viral signals. The complex regulation of HIV allows the virus to establish latency, then respond rapidly to various signals and synthesize high levels of viral proteins and virions, leading to the production and release of large numbers of progeny virus, the subsequent destruction of the infected cell, and the re-infection of large numbers of healthy CD4+ lymphocytes.

Antiretroviral Agents:

The field of antiretroviral chemotherapeutics developed in response to the need for agents effective against retroviruses, in particular EV. By the end of 2002, sixteen antiretroviral agents were approved by the FDA for treatment of HIV/AIDS. While there are many ways, in principle, in which an agent can exhibit anti-retroviral activity, all of these agents inhibit either the viral reverse transcriptase, or the viral protease. Highly active antiretroviral therapy (HAART) refers to a variety of drug ‘cocktails’, or combinations of three or more antiretroviral agents, that can potently suppress viral replication and prevent or delay the onset of AIDS (Mitsuya, H., and J. Erickson. 1999. Discovery and development of antiretroviral therapeutics for HIV infection., p. 751-780. In T. C. Merigan and J. G. Bartlet and D. Bolognesi (ed.), Textbook of AIDS Medicine. Williams & Wilkins, Baltimore). However, the ability to provide effective long-term antiretroviral therapy for HIV-1 infection has had only partial success, since 40 to 50% of those who initially achieve favorable viral suppression to undetectable levels eventually experience treatment failure (Grabar et al., 2000. Factors associated with clinical and virological failure in patients receiving a triple therapy including a protease inhibitor. Aids. 14:141-9; Wit et al., 1999. Outcome and predictors of failure of highly active antiretroviral therapy: one-year follow-up of a cohort of human immunodeficiency virus type 1-infected persons. J Infect Dis. 179:790-8). Moreover, 10 to 40% of antiviral therapy-naive individuals infected with HIV-1 have persistent viral replication (plasma HIV RNA>500 copies/ml) under HAART (Gulick et al., 1997. Treatment with indinavir, zidovudine, and lamivudine in adults with human immunodeficiency virus infection and prior antiretroviral therapy. N Engl J Med. 337:734-9; Hammer et al. 1997. A controlled trial of two nucleoside analogues plus indinavir in persons with human immunodeficiency virus infection and CD4 cell counts of 200 per cubic millimeter or less. AIDS Clinical Trials Group 320 Study Team. N Engl J Med. 337:725-33; Staszewski et al., 1999. Efavirenz plus zidovudine and lamivudine, efavirenz plus indinavir, and indinavir plus zidovudine and lamivudine in the treatment of HIV-1 infection in adults. Study 006 Team. N Engl J Med. 341:1865-73) possibly due to transmission of drug-resistant HIV-1 variants (Wainberg, M. A., and G. Friedland. 1998. Public health implications of antiretroviral therapy and HIV drug resistance. JAMA. 279:1977-83). In addition, it is evident that with these anti-HIV drugs only partial immunologic reconstitution is attained in patients with advanced HIV-1 infection.

Drug Resistance:

The rapid emergence and spread of drug-resistant mutant strains of HIV is rendering current drugs ineffective, and is one major cause of treatment failure. Recent estimates are that over 75% of drug-experienced patients in North America harbor HIV that is resistant to one or more of the 16 FDA-approved antiretroviral agents used in multi-drug ‘cocktails’. Drug-resistant HIV accounts for up to 12% of new infections. Drug-resistant HIV strains emerge in individuals who are infected with a wild type strain of HIV and who are exposed to suboptimal doses of one or more antiretroviral agents (Burger, et al, Antivir. Ther., 1998). There are three major classes of antiretroviral agents: nucleoside reverse transcriptase inhibitors (NRTIs), non-nucleoside reverse transcriptase inhibitors (NNRTIs), and protease inhibitors (PIs). The initial strain of drug resistant HIV that is selected depends on the particular drug regimen, and often requires the replacement of one drug by another of the same class. However, over time the continued selection of new strains with multiple mutations often leads to class-specific drug-resistance and, eventually, to complete treatment failure. Cross-resistance to drugs of the same class is spreading at an alarmingly high rate (Erickson, et al, AIDS, 13:S189, (1999); Gulnik, et al, Vitam. Horm., 58:213 (2000); Menendez-Arias, et al, Trends Pharmacol. Sci., 23:381 (2002)).

Drug Side Effects:

Based on the well-accepted theory that drug resistance emerges as a result of low level replication in the presence of sub-optimal levels of a drug, it has become common practice in antiretroviral therapy to prescribe the maximum tolerable dose of every drug in the cocktail. Since HIV is a chronic and incurable infection, the requirement for daily dosing of antiretroviral drug cocktails at maximum dosages results in very high peak drug levels. This practice has led to an alarmingly high rate of life-threatening side effects due to the chronic toxicities of many of these drugs (for review see Tozser, et al, Ann. NY Acad. Sci. 946:145 (2001)). Some of the more serious side effects associated with HAART toxicity include liver problems, heart disease, and lipodystrophy (Chen, et al, J. Clin. Endocrinol. Metab., 87:4845 (2002); Holstein, et al, Exp. Clin. Endocrinol. Diabetes 109:389 (2001)). The combination of resistance and side effects result in poor adherence to drug regimens and, ultimately, to treatment failure rates of between 40-45% (Wit, et al, J. Infect. Dis. 179:790 (1999); Fatkenheuer, et al, AIDS 11:F113 (1997); Lucas, et al, Ann. Intern. Med. 131:81 (1999); Chen, et al, 41st Intl Conf Antimicrob. Agents Chemother., Abstract I-1914 (2001)). Thus, a substantial number of patients currently taking HAART will soon run out of therapeutic options.

The long-term benefits of HAART are limited by the dual problems of poor adherence and drug resistance. In addition to these problems, the prohibitively high costs of drug have severely limited access of the global HIV-infected population to HAART. Thus, there is an urgent need for new therapeutics that are 1) effective against wild type and drug resistant viruses, 2) safe and non-toxic, and, 3) relatively inexpensive to produce or, at least, to deliver. These requirements pose formidable challenges when added to the conventional issues of potency, pharmacology, safety, and mechanism of drug action (De Clercq, Clin Microbiol Rev. 10:674-93 (1997); Erickson et al., AIDS 13:S189-204 (1999)).

Fusion Inhibitors and their Limitations for Prolonged Therapy:

One attractive solution to the drug resistance problem is to develop drugs with different mechanisms of action than those currently on the market. There are many ways, in principle, in which an agent can exhibit anti-retroviral activity in cell culture. Inhibitors of EV with novel mechanisms of action have been reviewed by DeClerq, Curr Med Chem. 8:1543-72 (2001). Among these compounds, polypeptide inhibitors of HIV fusion (“anti-fusiogenic peptides”) have been shown to be effective in human clinical trials. HIV infects human lymphocytes and other cell types bearing the membrane-bound CD4 glycoprotein and a chemokine receptor. The initial step in HIV infection of a CD4-bearing cell is the recognition of the CD4 receptor by the HIV gp120 envelope protein, which is non-covalently associated with the viral membrane through the viral membrane-bound HW gp41 envelope protein. The gp41 protein, or “fusion protein”, contains several “fusiogenic” domains, including a fusion peptide and two self-associating helix-forming segments (the “N-helix” and “C-helix”).

Recognition and binding of gp120 protein to the CD4 and chemokine receptors triggers the unmasking of the fusiogenic domains, the insertion of gp41 into the cell membrane, and the self-association of the two helix-forming segments into a “hairpin” structure. The formation of the hairpin structure of gp41 is believed to be an essential step in the fusion of the viral and cell membranes, and is a slow process, requiring up to 30 min to complete. Membrane fusion events, while commonplace in normal cellular processes, are also involved in a variety of disease states, including, for example, the entry of enveloped viruses into cells, and the aberrant fusion of virus-infected cells with healthy cells, leading to the formation of syncytia, and the subsequent clearance, or death, of the cells. Peptides and small molecules are known to inhibit or otherwise disrupt membrane fusion-associated events, including, for example, inhibiting retroviral infection of target cells.

Numerous polypeptides have been described which inhibit the HIV infection of CD4 cells by interfering with the fusion reaction. Several of these so-called “anti-fusiogenic peptides” are derived from the native amino acid sequence of either of the two helix-forming segments of gp41 (Jiang et al, Curr. Pharmaceut. Design 8:563 (2002)). Polypeptides consisting of sequences from either the N- or C-helix-forming regions of gp41 exhibit antiviral activity in cell culture assays. X-ray crystal structures and NMR solution structures of various isolated, recombinant forms of HIV-1, HIV-2 and SIV fusion proteins show that they all form trimers that with an anti-parallel, helical bundle-type fold. The bundles consist of three sets of hairpins, each of which is formed by the antiparallel association between the two helix-forming segments from a single protein chain. The hairpins are arranged in such a way that the first, or N-terminal, helical segments are associated in a trimeric inner bundle, and the second, or C-terminal, segments interact with the grooves formed by two adjacent N-terminal helices. Thus, the hairpin structure appears to be a consequence of assembly into the quaternary structure of the trimer, as opposed to being the fundamental building block of the trimer.

Peptides from the C-terminal and N-terminal heptad repeat regions, including DP178 (Wild, et al., Proc. Natl. Acad. Sci. USA, 91:9770 (1994)), also known as T-20, C34 (Chan, et al, Proc. Natl. Acad. Sci. USA, 95:15613 (1998)), and DP 107 exhibit potent antiviral activity.

U.S. Pat. Nos. 6,013,263, 6,017,536 and 6,020,459 incorporated herein in their entirety, likewise disclose that the 36 amino acid peptide DP178 corresponding to amino acids 638 to 673 of gp41 from the HIV-1 isolate LAI (HIV-1 LAI), and the 38 amino acid peptide DP107 corresponding to amino acids 558-595 of gp41 from the HIV-1 LAI, both exhibit potent anti-HIV-1 activity. WO 00/06599 teaches the use of C34 to inactivate gp41, and thus, prevent or reduce HIV-1 entry into cells. They are postulated to bind to the trimeric coiled-coil, or core, structure of gp41 during the transient state, and thereby prevent binding of the endogenous C-helices. T-20, a 34-residue peptide, has been shown to effectively lower viral load in drug-experienced patients. This validates gp41 as a promising target for the development of new anti-HIV drugs. Unfortunately, the therapeutic delivery of T-20 is limited by its peptidic nature. T-20 has a short half-life of 1.8 hours (Kilby, et al, Nat. Med., 4:1302 (1998)), and needs to be administered by subcutaneous injection, twice a day. Injection-site inflammation is a common side-effect reaction, and the drug formulation and manufacturing challenges result in high cost of treatment. T-20 is also rendered ineffective through the selection of a number of single mutations that lead to drug resistance both in vitro and in vivo.

A backup FI, T-1249, is in Phase II clinical trials. This compound is an even longer peptide than T-20. Its chief advantage is that it is more potent and has a longer half-life than T-20. However, T-1249 still suffers from the requirement for daily injection, and drug resistant mutants are readily selected using this drug.

Like many polypeptides, both T-20 and T-1249 must be administered intravenously or subcutaneously, and, both exhibit a short half-life in vivo, primarily due to rapid serum clearance and peptidase and protease activity. These pharmacological limitations reduce the therapeutic effectiveness of these agents, while at the same time resulting in a high cost of treatment.

C34, like T-20 and T-1249, also suffers from a short half-life iin vivo, primarily due to rapid serum clearance and peptidase and protease activity. This in, turn greatly reduces its effective anti-viral activity.

It can be generally assumed that many of the anti-fusiogenic polypeptides and peptidomimetics described in the art will suffer from the same limitations as found with T-20 and T-1249 to the extent that they are of a similar size, on the order of 30-40 amino acids.

Current antiretroviral therapy is a trade-off between the development of life-threatening side effects and life-threatening drug resistance. There is, therefore, a general need of a method for providing antiretroviral agents at drug levels that will reduce the chronic toxicity of these agents without compromising their therapeutic effectiveness. As an example, there is a need for a method of prolonging the half-life of peptides like C34 in vivo without substantially affecting its anti-fusiogenic activity. There is also a need for developing an inhibitor that will be effective in the treatment of drug-resistant HIV infections, particularly infections due to viral strains that are resistant to T-20 and T-1249. Further, there is a need to develop agents that can prevent, or retard, the emergence of drug resistant HIV in the therapeutic setting of a wild type infection.

There is also a need for a method of prolonging the half-life of reverse transcriptase inhibitors and protease inhibitors, for example, such that these agents can be administered in dosages that will be less toxic on a long term basis. Such methods are likely to result in less expensive therapies since cumulative drug quantities required per patient per year will be lower than for current therapies.

In view of the foregoing problems, there exists a need for inhibitors against drug resistant HIV strains. Further, there exists a need for inhibitors against drug resistant HIV gp41. Further still, there exists a need for inhibitors of HIV that can prevent or slow the emergence of drug resistant HIV strains in infected individuals. Inhibitors with the ability to inhibit drug resistant HWV strains, and to slow the emergence of drug resistant strains in the setting of wild type HIV infections, are defined as “resistance-repellent” inhibitors.

There also exists a need for HIV fusion inhibitors with prolonged duration of action. Inhibitors with prolonged in vivo half-lives that possess durable suppression of viral replication in vivo are defined as “long-lasting” inhibitors.

It should be recognized that resistance-repellent inhibitors and long-lasting inhibitors each represent clear and unique advantages in the treatment of HIV/AIDS. It should also be recognized that the combination of these two properties in a single agent would represent a revolutionary advance in antiviral therapy. Inhibitors that are both resistance-repellent and long-lasting are defined as broad spectrum durable inhibitors.

Serum Albumin as a Prodrug:

A doxorubicin-albumin conjugate has been disclosed as an antineoplastic prodrug agent. (F. Kratz et al, J. Med. Chem. 2000, 43, 1253-1256). However, the conjugate was prepared with an acid sensitive linker that allows the drug to be released at the low pH values present in lysosomes and indosomes of tumor cells. The preparation of the conjugate was designed to avoid the ex vivo synthesis and characterization of drug albumin conjugate which was considered to be costly.

### SUMMARY OF THE INVENTION

The present invention relates to biologically active compounds that may be used to react with proteins to form covalently linked complexes wherein the resulting complexes are found to exhibit desirable biological activities in vivo. More specifically, the complexes are isolated complexes comprising a compound, such as an antiviral compound and a linking group, and the blood component is a protein such as albumin. The present invention also provides methods for achieving a desired activity in vivo, such as anti-viral activity, comprising administering to the bloodstream of a mammalian host the novel isolated complexes of the present invention.

In one embodiment, a pharmaceutical composition is provided that comprises a purified conjugate, such as an anti-viral complex, according to the present invention as an active ingredient. Pharmaceutical compositions according to the invention may optionally comprise 0.001%-100% of one or more conjugates, such as anti-viral complexes, of this invention. These pharmaceutical compositions may be administered or coadministered by various methods known in the art for administering biologically active agents to the bloodstream. In a preferred aspect of the invention, the compositions may be administered by injection. In another preferred aspect, the compositions may be administered by infusion. The composition may advantageously comprise a buffered saline solution of the conjugate.

In another embodiment, methods and compositions are provided for delivery of isolated conjugated complexes comprising biologically active agents, particularly therapeutic agents such as anti-viral agents, where the complexes comprising the agents have an extended half-life in the bloodstream as compared to non-conjugated agents.

The invention comprises using a biologically active compound covalently attached or linked to a linking group, the linking group comprising at least one chemically reactive moiety which is capable of forming covalent bonds with functionalities present on the protein. By preparing the isolated complexes before administration of the complexes into the blood of the host, particularly the bloodstream of the host, a biologically active complex is generated that maintain an effective therapeutic effect in the bloodstream for an extended period of time as compared to a non-conjugated biologically active agent.

In particular, the present invention provides for resistance-repellent, long-lasting and broad spectrum durable inhibitors of HIV gp41 and HIV, their compositions, methods of design, and uses thereof for treating drug-resistant HIV and wt infections in both salvage therapy and first-line therapy modalities.

In one embodiment, the invention provides resistance-repellent inhibitors of HIV gp41 that target wild type and drug-resistant mutant gp41 proteins, and that have antiviral activity against wild type and drug-resistant HIV strains. In particular, these compounds are active against wild type HIV strains that contain naturally-occurring polymorphisms in the sequence of gp41, and that contain mutations that confer resistance to T-20 and/or T-1249. In one embodiment, these inhibitors are peptide sequences that are related to peptide sequences of the N and C-terminal helical repeat regions of gp41.

In another embodiment, this invention relates to the design of broad spectrum durable (persistent) inhibitors of HIV gp41 that target wild type and drug-resistant mutant gp41 proteins, and that have antiviral activity against wild type and drug-resistant HIV strains. In particular, these compounds are active against wild type HIV strains that contain naturally-occurring polymorphisms in the sequence of gp41, and that contain mutations that confer resistance to T-20 and/or T-1249. The design of broad spectrum durable inhibitors of HIV gp41 relates to chemically reactive modifications of peptides exhibiting anti-viral and/or anti-fusiogenic activity such that the modified peptides can react with available functionalities on blood components to form stable covalent bonds. In one embodiment of the invention, the modified peptides comprise a reactive group which is reactive with amino groups, hydroxyl groups, or thiol groups on blood components to form stable is covalent bonds. In another embodiment of the invention, the reactive group can be a moiety, such as a maleimide, which is reactive with a thiol group on a blood protein, including a mobile blood protein such as albumin.

More specifically, the present invention provides broad spectrum durable gp41 inhibitors which are capable of reacting with thiol groups on a blood component, either in vivo or ex vivo, to form a stable covalent bond. In addition, the complexes formed from the methods disclosed herein are in themselves more stable and are longer acting than the un-modified compounds. These complexes formed from the present invention have an extended iii vivo half-life when compared with the corresponding un-modified compounds. The complexes of the invention is stable toward hydrolytic cleavage or degradation for a period of about 4 hours to about 120 days.

In a further embodiment, this invention relates to the design of bioconjugated compositions of broad spectrum durable inhibitors of HIV gp41 that are covalently linked to a mobile blood protein such as serum albumin in a manner such that the bioconjugated form of the inhibitor has antiviral activity against both wild type and drug-resistant HIV strains. In particular, these compounds are active against wild type HIV strains that contain naturally-occurring polymorphisms in the sequence of gp41, and that contain mutations that confer resistance to T-20 and/or T-1249. In one embodiment ofthis invention, the bioconjugates are formed using modified peptides that comprise a reactive group which is reactive with amino groups, hydroxyl groups, or thiol groups on blood components to form stable covalent bonds. In another embodiment of the invention, the reactive group can be a moiety, such as a maleimide, which is reactive with a thiol group on a blood protein, including a mobile blood protein such as albumin.

The invention also provides the compounds described above bound in a complex with wild type or drug resistant mutant forms of HIV-1 gp41.

The invention further provides pharmaceutical compositions, comprising an inhibitor as described above, together with a pharmaceutically acceptable additive, excipient, or diluent. The composition may further comprise an additional HIV gp41 inhibitor and/or an HIV protease inhibitor and/or an HIV reverse transcriptase inhibitor.

The invention fuirther provides methods of treating a patient suffering from HIV infection, comprising administering to the patient a pharmaceutical composition as described above.

In further embodiments, the present invention relates to biologically active compounds that may be used to react with proteins to form covalently linked complexes wherein the resulting complexes are found to exhibit renin inhibition activities in vivo. More specifically, the complexes are isolated complexes comprising a renin inhibitor and a linking group, and the blood component is a protein such as albumin. The present invention also provides methods for inhibiting renin activity in vivo comprising administering to the bloodstream of a mammalian host the novel isolated complexes of the present invention.

In one embodiment, a pharmaceutical composition is provided that comprises a purified renin inhibitor complex according to the present invention as an active ingredient. Pharmaceutical compositions according to the invention may optionally comprise 0.001%-100% of one or more renin inhibitors complexes of this invention. These pharmaceutical compositions may be administered or coadministered by various methods known in the art for administering biologically active agents to the bloodstream. In a preferred aspect of the invention, the compositions may be administered by injection. In another preferred aspect, the compositions may be administered by infusion.

In another embodiment, methods and compositions are provided for delivery of isolated conjugated complexes comprising biologically active agents, particularly therapeutic agents such as renin inhibitors, where the complexes comprising the agents have an extended half-life in the bloodstream as compared to non-conjugated agents.

The invention comprises using a biologically active compound covalently attached or linked to a linking group, the linking group comprising at least one chemically reactive moiety which is capable of forming covalent bonds with functionalities present on the protein. By preparing the isolated complexes before administration of the complexes into the blood of the host, particularly the bloodstream of the host, a biologically active complex is generated that maintain an effective therapeutic effect in the bloodstream for an extended period of time as compared to a non-conjugated biologically active agent.

Definitions:

Unless otherwise stated, the following terms used in the specification and claims shall have the following meanings for the purposes of this Application.

A “complex” as used herein, is a compound comprising a biologically active agent such as an anti-viral compound or a renin inhibitor, a linking group and a protein such as albumin.

“Derivative” means a compound that is derived from some other compound and usually maintains its general structure.

“Isolated” such as an isolated compound is a compound, such as a naturally occurring compound such as albumin, that is substantially separated from other components which accompany the compound in its natural state. “Isolated” as applied to a compound obtained from blood or blood plasma, means a compound, such as a particular biological component from blood protein or blood plasma, that is purified or isolated from other biological compounds or components in the blood or blood plasma before the compound is further conjugated with a biologically active agent such as an anti-viral agent or renin inhibitor or the like. The isolated compound exists in a physical milieu distinct from that in which it occurs in nature and/or has been completely or partially separated or purified from other components in nature prior to submitting the compound to a reaction with the biologically active agent. The isolated compounds or complex of the invention has the advantage of allowing more selective reaction or conjugation with the biologically active agents, such as an anti-viral agent or renin inhibitor or the like, of the present invention with minimum interference from reactions with undesired components of the blood or blood plasma.

“Linker” as used herein, refers to a linking group which links or attaches a biologically active compound AV with a protein Pr, such as albumin, to form a covalently bound complex comprising the biologically active compound, the linker, and the protein.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of inhibitors of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Protected derivatives” means derivatives of inhibitors in which a reactive site or sites are blocked with protecting groups. Protected derivatives are useful in the preparation of inlnbitors o anti-viral agents or in themselves may be active as inhibitors or anti-viral agents. A comprehensive list of suitable protecting groups can be found in T. W. Greene, Protecting Groups in Organic Synthesis, 3rd edition, John Wiley & Sons, Inc. 1999.

“Therapeutically effective amount” means that amount which, when administered to an animal for treating a disease, is sufficient to effect such treatment forthedisease.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the disease (i.e., arresting irther development of the pathology and/or symptomatology), or

(3) ameliorating the disease in an animal that is experiencing or displaying the pathology or symptomatology of the disease (i.e., reversing the pathology and/or symptomatology).

“Stable”: a conjugate is stable when it is not cleaved prior to binding to a target, and where the macromolecular component of the conjugate, such as albumin, is not substantially degraded prior to target binding. The macromolecule is not substantially degraded when, even though some protease cleavage may occur, the conjugate retains a molecular weight greater than about 50 kDa. The conjugate is considered intact when it retains a molecular weight of at least about 50 kDa.

“Substantially retains”: a conjugate substantially retains the activity ofthe pharmacologically active moiety when its activity is at least about 10% of the non-conjugated pharmacologically active moiety (and may be higher on a molar ratio). Typically the activity of the conjugate is 0.1 to 10 times the activity of the non-conjugated pharmacologically active, though further enhancements of activity may be observed.

“Pharmacologically inert”: with respect to a macromolecule used in a conjugate means that the molecule is non-toxic. The molecule may or may not have biological activity distinct from that of conjugate, thought it typically does not. “No biological activity” means that administration of non-conjugated carrier to subject does not produce any substantial perturbation in normal physiology of the subject.

“Pseudo-peptides” or “peptide mimetics” or “peptidomimetics” means modified peptides that are structural analogues of the peptide that are designed to mimic the structure, properties and activities of the peptide. The modified peptides have improved biological and finctional activities compared to the unmodified peptide due to their higher level of resistance to enzymatic degradation while exhibiting the same or improved biological activities.

For all peptides described herein, except where specifically indicated otherwise, the peptide sequence will be understood to indicate N- protected derivatives such as N-acetyl compounds, and C-amide derivatives, as well as the free amino and free carboxy compounds.

### BRIEF DESCRIPTION OF THE DRAWINGS

FIG. 1 shows fusion inhibitor peptides of the invention.

FIG. 2 shows the pharmacolinetics of unconjugated (SPI-30014Q) vs HSA-conjugated (SPI30014HSA) fusion inhibitor peptide in Sprague-Dawley rats.

FIG. 3 shows the pharmacokinetics of unconjugated (SPI-70038Q) vs HSA-conjugated HIV (SPI-70038HSA) fusion inhibitor peptide in Sprague-Dawley rats.

FIG. 4 shows the pharmacokinetics of Reactive peptide (SPI-30014) vs HSA-peptide conjugates (SPI-30014HSA) in Sprague-Dawley rats.

FIG. 5 shows the pharmacokinetics of reactive peptide (SPI-70038) vs HSA peptide conjugate (SPI-70038HSA) in Sprague-Dawley rats

### DETAILED DESCRIPTION OF TEHE INVENTION

The invention provides conjugates, including purified conjugates, of a biologically or pharmacologically active moiety and a macromolecule, that have superior pharmacological properties and that produce sustained biological activity when administered to a mammalian subject. In particular, the invention provides an isolated compound where a pharmacologically active moiety is covalently conjugated to a pharmacologically inert macromolecular carrier, where the linkage between the pharmacologically active moiety and the carrier is stable iin vivo, where the intact compound substantially retains the pharmacological activity of the pharmacologically active moiety, and where the active half-life of the compound when administered to a mammal is at least about twice that of the unconjugated pharmacologically active moiety. The carrier advantageously is HSA and the conjugates are used for methods of human therapy and prophylaxis.

Previous work has described conjugates that contain biologically active molecules linked to macromolecular moieties. A significant body of work describes, for example, conjugates of the cytotoxic agents doxorubicin and methotrexate to human serum albumin (HSA) . In these methodologies the rationale has been to link the cytotoxic agent to the HSA via a labile linkage that is severed upon uptake of the conjugate at the desired site iin vivo. Typically, the labile linkage is acid sensitive and is severed upon cellular uptake into the acidic environment of the endosome. In this manner, therefore, the conjugate was viewed in essence as a prodrug moiety that was required to be degraded to release the biologically active molecule.

Other work has described methods of injecting “activated” biologically active molecules into the bloodstream of subjects, where the activated moiety is assumed to bind to one or more blood proteins, such as HSA, preparing a conjugate in situ. This method has numerous drawbacks, including inability to control the composition and yield of the conjugate with concomitant uncertainty regarding the dosing of the conjugate. Moreover, many activated biologically active molecules have limited aqueous solubility and are chemically unstable, which not only makes handling and administration of the activated moiety problematic, but results in further uncertainty regarding in vivo reactivity and dosing.

Still other work has described preparation of fusion proteins containing HSA and a protein of interest. These methods are, of course, limited to conjugates of molecules that can be made by recombinant DNA methods. Also, the site of attachment of the peptide or protein to the HSA is limited to either the C- or N-terminus of the HSA and the nature of the attachment is necessarily via a peptide bond.

Typically, the non-covalent binding or adsorption of a drug to a blood protein component is viewed as a disadvantage to the extent that protein binding reduces the concentration of free drug available for pharmacological activity. However, the present inventors surprisingly have found that conjugates of peptide and non-peptide biologically active molecules linked to macromolecular moieties that are prepared ex vivo and that carry non-labile linkers provide valuable advantages over methods and compositions that previously have been described. In particular, such “cloaked” compositions (where the macromolecule “cloaks” the biologically active moiety) prepared ex vivo in which biologically active molecules are covalently linked to HSA have been found to have unexpectedly superior pharmacological and, in particular, pharmacokinetic properties, to previously known compositions.

Specifically, the present inventors have found that ex vivo conijugation of a biologically active moiety to a macromolecule such as HSA produces a highly soluble conjugate that can be purified and administered in tightly controlled dosage. The cloaked conjugate is biologically active as the conjugate, i.e. it does not act as a prodrug that releases the biologically active moiety from the conjugate and cleavage of the conjugate is not required for biological activity. Moreover, once administered to a subject the conjugate has a surprisingly long ini vivo half-life, has excellent tissue distribution and produces sustained activity corresponding to the activity of the biologically active moiety of the conjugate. In addition, assays using radiolabeled conjugate show that essentially all of the administered conjugate can be accounted for in vivo following administration to the subject, In comparison, in assays using radiolabeled active moiety, where the conjugate presumably is formed in situ, up to 50% of the active moiety administered to the subject cannot be accounted for. In addition, chemical conjugation between the biologically active moiety and the macromolecule permits variation in the length and nature of the linker.

Advantageously, the biologically active moiety and the macromolecule are linked in an approximately 1:1 ratio, to avoid “haptenization” of the biologically active moiety and generation of an immune response to the conjugate. Moreover, the biologically active moiety is advantageously appended to a single site in the macromolecule. For example, selective linkage to the unusually reactive cysteine 34 (C34) of HSA may be used. Methods for selective linkage to C34 using, for example, a maleimide containing linker, are known in the art. Suitable linkers are commercially available from, for example, Pierce (Rockford, Ill.).

In the event that more than one molecule of biologically active moiety is linked to the macromolecule, this is advantageously achieved via a “multivalent” linker that is attached to a single point of the macromolecule. For example, a linker can be appended to C34 of HSA that permits attachment of a plurality of biologically active moieties to the linker. Multivalent linkers are known in the art and can contain, for example, a thiophilic group for reaction with C34 of HSA, and multiple nucleophilic (such as NH or OH) or electrophilic (such as activated ester) groups that permit attachment of a plurality of biologically active moieties to the linker.

Advantageously the biologically active moiety is relatively small in size compared to the macromolecule to maximize the “cloaking” effect of the macromolecule, such as HSA. Although the skilled artisan will recognize that precise upper limits cannot be placed on the size of the biologically active moiety, it is believed that molecules with molecular weights less than 50 kD, less than 10 kD, and advantageously less than 7.5 kD or 5 kD can be used.

Methods of linking the biologically active moiety to the macromolecule are known in the art and are discussed, for example, in WO00/76550, which is hereby incorporated by reference in its entirety. Such methods are also discussed in more detail below with respect to conjugates of fusion inhibitor peptides and renin inhibitors. The skilled artisan will recognize that the conjugation methods discussed for the viral fusion inhibitors and renin inhibitors are generally applicable to a panoply of biologically active moieties, and are merely illustrative of the present technology. Similarly, methods for purifying the conjugates (if necessary) are known in the art. For example, excess biologically active moiety can be removed by dialysis of the conjugate, which can be further purified by reversed-phase HPLC, ion exchange chromatography, and/or size exclusion chromatography or the like.

Biologically Active Compounds

The present invention encompasses a wide variety of biologically and/or pharmacologically active moieties that may be “cloaked” using the methods described herein. In addition to the renin inhibitors and viral fusion inhibitors exemplified below, essentially any molecule for which enhanced pharmacological properties, and in particular, sustained activity, are desirable, can be cloaked. Examples of groups that can be cloaked include peptide and non-peptide molecules. Specific examples include compounds having metabolic effects, such as cholesterol lowering and blood-pressure lowering compounds, compounds for treatment of neurological disorders (where the conjugate can be optionally administered directly into the CNS) including wound healing agents, antibiotics (incuding anti-infectives), anti-oxidants, chemotherapeutic agents, anti-cancer agents, anti-inflammatory agents, and antiproliferative drugs. Examples of these molecules are well known in the art and include, merely for illustrative purposes:

Inhibitors of matrix metalloproteinases (MMPs), antagonists of the urokinase receptor, inhibitors of urokinase, erb-2 receptor antagonists, TRAIL receptor antagonists, antiangiogenic peptides, opiods and anti-nociceptive analogs for pain, antihypertensives such as renin inhibitors; angiotensin receptor antagonists;

natriuretic peptide derivatives, antivirals such as interferons (including alpha and beta interferon for treatment of hepatitis C); cyanovirin derivatives, compounds for treatment of metabolic disorders such as insulin, bacterial and yeast extracellular virulence factors such as proteinases, bacteriophage lysins, viral entry and fusion inhibitors (for viruses such as herpes viruses, such as HSV-2-genital herpes, viral glycoprotein D-nectin-2 interaction, HCV-E1,E2 glycoprotein interaction with CD81, LDL receptor and other cell-specific and liver specific cofactors, malarial plasmepsins, schistosomal aspartic proteinases, chaperones that stabilize proteins 10 causing protein-misfolding diseases or drugs that downregulate production of these proteins and that may be used for treatment of diseases such as Alzheimer's disease (for example secretase inhibitors).

ACE-inhibitors, α- and β-adrenergic agonists agonists and antagonists, adrenocorticoids, hormones, aldose reductase inhibitors, aldosterone antagonists, 5-αreductase inhibitors, analgesics, anesthetics, anorexics, anthelnintics, antiacne agents, antiallergic agents, antialopecia agents, antiamebic agents, antiandrogen agents, antianginal agents, antiarrhythmic agents, antiarteriosclerotic agents, antiarthritic/antirheumatic agents, antiasthmatic agents, antibacterial agents, aminoglycoside antibiotics, ansamycins, antibiotics and antobacterials such as β-lactams, lincosamides, macrolides, polypeptides, tetracyclines, 2,4-diaminopyrimidines, nitrofurans, quinolones and analogs, sulfonamides, sulfones, antibiotics, anticholelithogenic agents, anticholesteremic agents, anticholinergic agents, anticoagulant agents, anticonvulsant agents, antidepressant agents, hydrazides/hydrazines, pyrrolidones, tetracyclics, antidiabetic agents, biguanides, hormones, sulfonylurea derivatives, antidiarrheal agents, antiduretic agents, antidyskinetic, antieczematic, antiemetic agents, antiepileptic agents, antiestrogen agents, antifibrotic agents, antifiatulent agents, antifungal agents, antiglaucoma agents, blood brain barrier peptides (BBB peptides), RGD peptides, glucagon-like peptides, antigonadotropin, antigout, antihemorrhagic and antihistaminic agents; tricyclic antidepressants, antihypercholesterolemic, antihyperlipidemic, anthyperlipidemic and antihyperlipoproteinemic agents, aryloxyalkanoic acid derivatives, bile acid sequesterants, HMG-CoA reductase inhibitors, nicotinic acid derivatives, thyroid hormones/analogs, antihyperphosphatemic, antihypertensive agents, arylethanolamine derivatives, arloxypropanolamine derivatives, benzothiadiazine derivatives, n-carboxyalkyl derivatives, dihydropyridine derivatives, guanidine derivatives, hydrazines/phthalazines, imidazole derivatives, quaternary ammonium compounds, quinazolinyl piperazine derivatives, reserpine derivatives, sulfonamide derivatives, antihyperthyroid agents, antihypotensive agents, antihypothyroid agents, anti-inflammatory agents, aminoarylcarboxylic acid derivatives, arylacetic acid derivatives, arylbutyric acid derivatives arylcarboxylic acids (including arylpropionic acid derivatives), pyrazoles, pyrazolones, salicylic acid derivatives, thiazinecarboxamides, antileprotic, antileukemic, antilipemic, antilipidemic, antimalarial, antimanic, antimethemoglobinemic, antimigraine, antimycotic, antinauseant, antineoplastic and alkylating agents, antimetabolites, enzymes, androgens, antiadrenals, antiandrogens, antiestrogens, progestogens, uroprotective, antiosteoporosis agents, antipagetic, antiparkinsonian, antiperistaltic, antipheochromocytoma, antipneumocystis, antiprostatic hypertrophy, antiprotozoal, antiprozoal, antipruritic, antipsoriatic and antipsychotic agents, butyrophenes, phenothiazines, thioxanthenes, antipyretic, antirheumatic, antirickettsial, antiseborreheic and antiseptic/disinfectant agents, antispasmodic,antisyphilitic, antithrombotic, antitubercular, antitumor, antitussive, antiulcerative, antiurolithic, antivenin, and antivertigo agents, purines/pyrimidinomes, antianxiolytics, arylpiperazines, benzodiazepine derivatives, carbamates, astringent, benzodiazepine antagonist, beta-blocker, bronchodilator, ephedrine derivatives, calcium channel blockers, arylalkylamines, dihydropyridine derivatives, piperazine derivatives, calcium regulators, calcium supplements, cancer chemotherapy agents, capillary protectants, carbonic anhydrase inhibitors, cardiac depressants, cardiotonic, cathartic, cation-exchange resin, cck antagonists, central stimulants,cerebral vasodilators, chelating agents, cholecystokinin antagonists, choleitholytic agents, choleretic agents, cholinergic agents, cholinesterase inhibitors, cholinesterase reactivators, cns stimulants, cognition activators, contraceptives, agents to control intraocular pressure, coronary vasodilators, cytoprotectants, dopamine receptor antagonists, ectoparasiticides, emetics, enzymes, digestive agents, mucolytic agents, penicillin inactivating agents, proteolytic agents, enzyme inducers, estrogen antagonists, gastric proton pump inhibitors, gastric secretion inhibitors, α-glucosidase inhibitors, gonad-stimulating principles, gonadotrophic hormones, growth hormone inhibitor, growth hormone releasing factor, growth stimulant, hematinic, hemolydic, demostatic, heparin antagonist, hepatoprotectant, histamine h.sub.1-receptor antagonists, histamine H2-receptor antagonists, immunomodulators, immunosuppressants, inotrophic agents, keratolytic agents, lactation stimulating hormone, lipotrophic agents, mineralocorticoids, minor tranquilizers, miotic agents, monoamine oxidase ihibitors, mucolytic agents, muscle relaxants, mydriatic agents, narcotic agents; narcotic antagonists, neuroleptic agents, neuromuscular blocking agents, neuroprotective agents, NMDA antagonists, nootropic agents, NSAID agents, ovarian hormones, oxytocic agents, GP-41 peptides, insulinotropic peptides parasympathomimetic agents, pediculicides, pepsin inhibitors, peripheral vasodilators, peristaltic stimulants, pigmentation agents, plasma volumne expanders, potassium channel activators./openers, pressor agents, progestogen, prolactin inhibitors, prostaglandin/prostaglandin analogs, protease inhibitors, proton pump inhibitors, reverse transcriptase inhibitors, scabicides, sclerosing agents, sedative/hypnotic agents, serotonin receptor agonists, serotonin receptor antagonists, serotonin uptake inhibitors, skeletal muscle relaxants, somatostatin analogs, spasmolytic agents, stool softeners, succinylcholine synergists, sympathomimetics, thrombolytics, thyroid hormone, thyroid inhibitors, thyrotrophic hormone, uricosurics, vasodilators, vasopressors, and vasoprotectants.

Antiviral Compounds and Complexes:

The present invention also provides anti-viral compounds that have prolonged or sustained activity for the treatment of viral disease. The invention also provides methods of treating viral diseases using these compounds. The compounds of the present invention have increased stability in vivo and a reduced susceptibility to degradation, for example by peptidase or protease degradation. As a result, the compounds of the present invention may be administered less frequently than presently available anti-viral compounds. The compounds can be used, e.g., as a prophylactic against and/or treatment for infection of a number of viruses, including human immunodeficiency virus (HIV), human respiratory syncytial virus (RSV), human parainfluenza virus (HPV), measles virus (MeV) and simian immunodeficiency virus (SIV).

The compounds of the invention achieve their sustained activity by covalent linkage to at least one blood component, or to a variety of different blood components. This linkage can be carried out inl vivo or in vitro. When the linkage is carried out in vitro, the compound may optionally be further purified, by filtration for example, prior to administration to a patient. For peptide compounds composed of naturally occurring amino acids, the covalent linkage can be achieved either by chemical means, for example by using a suitable cross-linking agent, or by preparation of a fusion protein with the blood component. For non-peptide compounds, or compounds containing non-naturally occurring amino acids, the linkage may be achieved by chemical means. The skilled artisan will be aware of blood components that are suitable for use in the present invention. In a particular embodiment, the blood component is human serum albumin, and in another embodiment the blood component is a human or humanized antibody, antibody fragment or antibody derivative. The antibody, antibody fragment or antibody derivative may optionally be an antibody, antibody fragment or antibody derivative that specifically binds a blood component, such as human serum albumin.

Anti-viral Compounds

The compounds of the present invention include compounds having anti-viral activity that can be conjugated to a blood component without a significant loss of anti-viral activity. In the context of the present invention, a significant loss of anti-viral activity refers to the situation where the anti-viral activity of the conjugated compound is reduced to the extent that the dosage of the compound must be increased by at least a factor of 10 in molar terms in order to obtain suitable in vivo activity.

Compounds suitable for use in the present invention include, but are not limited to: peptide inhibitors of viral fusion, nucleoside and nucleoside analog, non-nucleoside and non-nucleoside analog and nucleotide and nucleotide analog inhibitors of viral enzymes, inhibitors of viral proteases, and chemokine co-receptor blockers that inhibit viral entry into cells. Examples of each of these compounds are known in the art.

Non-limiting representative compounds include, for example:

nucleoside analogs, such cytosine-arabinoside, adenine-arabinoside, iodoxyuridine and acyclovir:

nucleoside and nucleotide reverse transcriptase inhibitors (NRTIs), such as AZT, ddI, ddC, d4T, 3TC, abacavir, tenofovir, emtricitabine, amdoxovir, dOTC, and d4TMP;

non-nucleoside reverse transcriptase inhibitors (NNRTIs), such as nevirapine, delaviridine, efavirenz; thiocarboxanilide UC-781, capravirine, SJ-3366, DPC 083, and TMC 125/R165335;

protease inhibitors (Pls), which include saquinavir, ritonavir, indinavir, nelfinavir, amprenavir, lopinavir, atazanavir, mozenavir, tipranavir, and TMC-114;

virus adsorption inhibitors, such as cosalane derivatives and cyanovirin-N co-receptor antagonists, for example, TAK-779 and AMD3100

viral fusion inhibitors, for example pentafuside T-20, betulinic acid, R170591, VP-14637, and NMS03

and viral uncoating inhibitors such as azodicarbonamide.

Other antiviral compounds that can be used include lamivudine, famciclovir, lobucavir and adefovir, Ribavirin, integrase inhibitors (diketo acids), transcription inhibitors (temacrazine, flavopiridol), viral uncoating inhibitors (pleconaril); RNA replicase inhibitors (VP-32947); DNA polymerase inhibitors (A-5021, L- and D-cyclohexenylguanine); bicyclic furopyrimidine analogues; cidofovir; neuraminidase inhibitors (zanamivir, oseltamivir, RWJ-270201); adefovir dipivoxil; N-glycosylation inhibitors (N-nonyl-deoxynojirimycin); and, IMP dehydrogenase inhibitors and S-adenosylhomocysteine hydrolase inhibitors.

For the treatment of HIV infection in particular, compounds can be used that inhibit:

viral adsorption, through binding to the viral envelope glycoprotein gp120 (polysulfates, polysulfonates, polycarboxylates, polyoxometalates, polynucleotides, and negatively charged albumins);

viral entry, through blockade of the viral coreceptors CXCR4 (i.e., bicyclam (AMD3100) derivatives) and CCR5 (i.e., TAK-779 derivatives);

virus-cell fusion, through binding to the viral envelope glycoprotein gp41 (T-20, T-1249);

viral assembly and disassembly, through NCp7 zinc finger-targeted agents (2,2′-dithiobisbenzamides (DIBAs), azadicarbonamide (ADA));

proviral DNA integration, through integrase inhibitors such as 4-aryl-2,4-dioxobutanoic acid derivatives; and

viral MRNA transcription, through inhibitors of the transcription (transactivation) process (flavopiridol, fluoroquinolones).

The Linkers L1 and L2:

A variety of different linkers or linking groups L1 and L2 may be used to link the blood component with the anti-viral agent. The linking groups may be divalent or polyvalent. For example, in the complex of Formula I, L1 may be n-valent where it is attached to Pr, and m-valent where it attaches to AV where m and n are integers as defined above. Similarly, in the complex of Formula U, L2 may be o-valent where it is attached to Pr and p-valent where it is attached to AV, where o and p are as defined above. Non-exclusive examples of functional groups that may be present in a linking group include compounds that have a hydroxyl groups, such as N-hydroxysuccinimide, N-hydroxysulfosuccinimide, and other compounds such as maleimide-benzoyl-succinimide, maleimido-butyryloxy succinimide ester, maleimidopropionic acid, N-hydroxysuccinimide, isocyanate, thioester, thionocarboxylic acid ester, imino ester, carbodiimide, anhydride, or ester.

In addition, certain linking groups having functional groups such as carboxylate, acid halide, azido, diazo, carbodiimide, anhydride, hydrazine, aldehydes, thiols, or amino group may be used to form amides, esters, imines, thioethers, disulfides, substituted anines, or the like. Other specific examples of functional groups that may be employed include acyloxymethylketones, aziridines, diazomethyl ketones, epoxides, iodo-, bromo- or chloroacetamides, α-haloesters, chaloketones, sulfoniums, chloroethylsulfides, O-alkylisoureas, alkyl halides, vinylsulfones, acrylamides, vinylpyridines, organometallic compounds, aryldisulfides, thiosulfonates, aldehydes, nitriles, α-diketones, α-ketoamides, α-ketoesters, diaminoketones, semicarbazones, and dihydrazides.

The nature and type of compounds that may be selected as the linker depends on the type of reactions, the relative reactivities, selectivities, reversibility and stability characteristics that are desired among the anti-viral agents, the linker and the functional groups on albumin or the blood component. For example, certain reactions that form the conjugate complex arise from an alkylation reaction, a Michael type reaction, an addition-elimination reaction, an addition to sulfur, carbonyl, or cyano groups, or the formation of a metal bond.

Typically, the covalent bond that is formed from these reactions are stable during the active lifetime of the anti-viral agent. In one embodiment, the covalent bond that is formed in these complexes remain stable unless the biologically active subunit is intended to be released at the active site.

The linkers may comprise of compounds having bifunctional or polyfunctional groups that are available for linking a protein such as albumin to multiple anti-viral agents or for linking multiple albumins to a single anti-viral agent. In a particular preferred embodiment, the linker comprises polyfunctional groups that link a HSA to one or more anti-viral agents. In one embodiment, linking compounds as used herein include any compounds that can link the anti-viral agent to the protein in a single step. In another embodiment, the linking compounds are linked to the anti-viral agent first to form a inhibitor-linker intermediate that can be further reacted with the protein. In another embodiment, the linking compounds are reacted with the protein first to form a protein-linker intermediate that can be further reacted with the anti-viral agent. In each of the above permutations, optionally, the linked compounds may be further purified and/or isolated before submitting to further reactions to form the complex of Formula I or Formula II.

Non-exclusive examples of such polyfinctional compounds include compounds having at least one functional group selected from the group consisting of azidobenzoyl hydrazide, N-[4-(p-azidosalicylamino)butyl]-3′-[2′-pyridyldithio)propionamide), bis-sulfosuccinimidyl suberate, dimethyl adipimidate, disuccinimidyl tartrate, N-y-maleimidobutyryloxysuccinimide ester, N-hydroxy sulfosuccinimidyl-4-azidobenzoate, N-succinimidyl [4-azidophenyl]-1,3′-dithiopropionate, N-succinimidyl[4-iodoacetyl]aminobenzoate, glutaraldehyde, and succinimidyl 4-[N-maleimidomethyl]cyclohexane-1-carboxylate.

Any linker or linking group that is convenient for use and subject to standard chemical transformations, or linkers that form compounds that are physiologically acceptable at the desired dosages, and are stable in the bloodstream for the desired period of time, may be employed. The linking group may be aliphatic, alicyclic, aromatic, heterocyclic, or combinations thereof. Examples of groups that may be employed as a linking group include alkylenes, arylenes, aralkylenes, cycloalkylenes, polyethers and the like. In a particular embodiment, polyfunctional polyethylene glycol (PEG) and their derivatives may also be employed as linkers.

The linking groups may have at least one atom in the linking chain, more preferably between 1 and 200 atoms in the chain, most preferably between 2 and 50 atoms in the chain. The atoms in the chain can be linear or the chain can be part of one or more rings, each substituted or unsubstituted, and the chain may include carbons or heteroatoms selected from the group consisting of O N, P and S. The rings may be aliphatic, heterocylic, aromatic or heteroaromatic or mixtures thereof, each substituted or unsubstituted. In some embodiments, amino acids or peptides or amino acids employed with mixtures of the above may be used as a linking group.

In one embodiment, L1 is absent and AV is attached directly to Pr. In another embodiment, L2 is absent and AV is attached directly to Pr.

In another embodiment for the complex of Formula I, L1 is a linking group that is capable of linking more than one AV to one Pr, for example, where m is 2 or more. In one embodiment, m is 1, 2 or 3 and n is 1-30. In one preferred embodiment for the complex of Formula I, Pr is albumin and n is 1. In another particular embodiment, Pr is albumin, AV is an anti-viral agent, and n is 2 - 25.

In another embodiment for the complex of Formula II, L2 is a linking group that is capable of linking more than one Pr to one AV, for example, in the case where o is 2 or more. In one embodiment, Pr is albumin, AV is an anti-viral agent, o is 1, 2 or 3 and p is 1-5.

In another embodiment, the linking group may be absent in cases where the inhibitor, such as an anti-viral agent, can be reacted directly with a protein, optionally using a catalyst or coupling agent, such that the complex that is formed comprises only of the anti-viral agent that is directly attached to the protein. An example of such a direct coupling reaction is a mixed anhydride activated coupling reaction of a carboxylic acid followed by the coupling reaction of the intermediate mixed anhydride.

The Protein Component Pr:

Various blood components may be used to prepare the isolated complexes of the present invention. Naturally occurring blood components include blood proteins, which include red blood cells, and immunoglobulins, such as IgM and IgG, serum albumin, transferrin, p90 and p38. In a preferred embodiment, the blood component or blood protein is albumin. More preferably, the albumin is a protein human serum albumin (HSA).

The albumin used in the present invention may also be recombinant albumin. For example, the recombinant human albumin may be produced by transforming a microorganism with a nucleotide coding sequence encoding the amino acid sequence of human serum albumin.

Generally, there exists a very broad range of different methods available for the isolation of compounds from blood or blood plasma that provide a very broad range of final purities, and yields of the product. Albumin is the main protein present in blood plasma, and may be extracted from blood, for examples as disclosed in JP 03/258 728, EP 428 758, EP 452 753, and U.S. Pat. No. 6,638,740 and references cited therein. Further examples of non-exclusive methods for the isolation of various compounds may be based on selective reversible precipitation, ion exchange chromatography, protein affinity chromatography, hydrophobic chromatography, thiophilic chromatography (J. Porath et al; FEBS Letters, vol. 185, p.306, 1985; K. L. Knudsen et al, Analytical Biochemistry, vol 201, p.170, 1992), and various resin matrices (WO 96/00735; WO 96/09116). Certain blood components of established purity are commercially available.

Preparation of Linked Compounds AV-L1 and AV-L2:

In one embodiment, the linked compounds AV-L1 or AV-L2 of the present invention may be prepared and used in the conjugation with albumin without further purification and/or isolation. The purity of the linked compounds will depend on the nature of the linker, the nature of AV, and the type of reaction and reaction conditions employed to attach AV to the linker. In another particular embodiment, the unpurified linked compounds are prepared and obtained with a purity of at least 90%, preferably at least 95%, more preferably at least 97%, and most preferably at least 98%.

In a particular embodiment, the present invention relates to methods for the preparation of the isolated linked compounds, that is, AV-L1 or AV-L2. In a preferred embodiment, the isolated linked compounds AV-L1 and AVL2 are anti-viral agents that are attached to a linker. In one embodiment, the isolated linked compounds may be purified before conjugating with Pr. In another particular embodiment, the linked compounds AV-L1 or AV-L2 are isolated and purified to a purity of at least 95%, preferably at least 97%, more preferably at least 98%, and most preferably at least 99% or more.

The linked compounds may be prepared using standard methods known in the art of chemical synthesis. The compounds may be purified using standard methods known in the art, such as by column chromatography or HPLC to provide purified products suitable for in vivo applications. The linked compounds may be further conjugated with a protein, such as albumin to form the complex of Formulae I and II.

Covalent Linkage to Blood Components

Suitable blood components for use in the present invention are known in the art. Human serum albumin (“HSA”) is a predominant component of human blood and is particularly suited for use in the present invention. In particular, HSA has an exposed surface cysteine residue that provides a reactive thiol moiety for covalent linkage of anti-viral compounds to the protein. Activated linkers that are particularly suited for linkage to thiols include unsaturated cyclic imides such as maleimides, cLhalo esters, such as α-iodo- and α-bromo acetates, and vinyl pyridine derivative. Suitable activated linkers are commercially available from, for example, Pierce Chemical (Rockford, Ill.). Methods for preparing suitable activated compounds for linking to HSA are known in art. See for example, U.S. Pat. No. 5,612,034, which is incorporated herein in its entirety.

In one variation of the present invention, the linker is specifically linked to the thiol group of cysteine 34, and maybe formed via a nucleophilic reaction of the thiol group on an electrophilic group of the linker.

Moreover, the gene for HSA has been cloned, which permits the ready preparation of fusion proteins containing HSA. These fusion proteins, which have therapeutic applications, include, but are not limited to a polypeptide, an antibody, or a peptide, or fragments and variants thereof, filsed to a blood component. The fusion proteins exhibit extended shelf-life andlor extended or therapeutic activity. Methods of making fusion proteins are known in the art. See, for example, WO01/79271 and WO01/79258, the contents of which are hereby incorporated by reference in their entirety. The preparation of fusion proteins is useful for preparing persistent derivatives of anti-viral peptides.

Another blood component that is suitable for linkage to the anti-viral compounds is an immunoglobulin (“Ig”) molecule. Igs are persistent and are present in relatively high concentration in the blood. For inz vitro coupling, Igs have the advantage of being readily stable and readily isolated, and methods of making Ig conjugates are well known in the art. Moreover, Ig genes may readily be cloned and recombinant Ig and Ig fusion proteins prepared. Methods for obtaining fully human Igs are well known in the art. See for example, U.S. Pat. Nos. 5,969,108 and 6,300,064, the contents of which are hereby incorporated by reference in their entirety. In addition, phage display methods for selecting Igs having a particularly desired binding activity, for example, for binding to HSA, are well known in the art. See U.S. Pat. Nos. 5,885,793, 5,969,108 and 6,300,064. In the context of the present invention, an Ig refers to any suitable immunoglobulin or immunogolobulin derivative known in the art, and includes, for example, whole IgG, IgM, Fab fragments, F(ab′)2 fragments, and single chain Fv fragments.

Other blood components suitable for use in the present invention include transferrin, ferritin, steroid binding proteins, thyroxin binding protein, and α-2-macroglobulin.

For peptides, activated linkers may be coupled to reactive side chain residues, such as lysine side chains. For example, a linker containing an active ester moiety and a maleimide moiety can be selectively reacted at the active ester (such as an N-hydroxysuccinimidyl ester) via lysine side chains or at the N-terminus of the peptide. For non-peptidyl anti-viral compounds, the skilled chemist readily can recognize nucleophilic (or electrophilic) atoms or groups on the compound that can selectively react with a suitable linking moiety, such as an active ester. Suitable nucleophilic moieties include, but are not limited to, amino and hydroxyl groups. For example, for nucleoside and nucleotide analogs, hydroxyl groups can act as nucleophiles for the coupling reaction. For nucleotides, coupling also can be achieved by formation of, for example, phospho esters. Similar strategies can be used in other anti-viral compounds, for example in protease inhibitors and other enzyme inhibitors, coupling can be achieved using nucleophilic groups that are distal from the enzyme active site.

Both natural and recombinant HSA and human Igs are commercially available and are suitable for use in the present invention.

The antiviral compounds can be prepared using synthetic methods that are well known to the skilled chemist. For example, peptides can be prepared using well-known techniques of solid-phase peptide synthesis. See, for example, Solid Phase Peptide Synthesis, 2nd Ed., Pierce Chemical Company, Rockford, Ill., (1984). Similarly, peptides fragments may be synthesized and subsequently combined or linked together to form the desired sequence.

Other compounds may be prepared using methods known in the art, or by straightforward variations on known methods.

Preparation of Linked Compounds Pr-L1 and Pr-L2:

For certain applications of the present invention, the compounds as represented by Pr may be albumin, may be used as obtained from commercial sources without further purification or isolation, to prepare the linked compounds Pr-L1 and Pr-L2. In a particular embodiment, Pr is HSA. In another embodiment, the albumin may be further purified using various methods known in the art as disclosed herein.

In one embodiment, the linked compounds Pr-L1 and Pr-L2 may be prepared by treating a linker L1 or L2, which may be derivatized or activated, with Pr, in a solution and monitoring the reaction mixture until the reaction is substantially complete. In a particular preferred embodiment, Pr is a protein. In another preferred embodiment, the protein is HSA or recombinant HSA.

In another preferred embodiment, the linked compounds Pr-L1 or Pr-L2 obtained are substantially pure; that is, the linked compounds are obtained with a purity of at least 10%, preferably at least 30%, and more preferably at least 50%. Where the Pr is HSA or recombinant HSA, components that may be present with the linked compounds may comprise of unreacted HSA and various biological components that are present in the HSA starting material. Preferably, the HSA or recombinant HSA is at least 10% pure on a dry matter basis.

An excess of HSA or HSA related biologically materials present with the linked compounds will not significantly interfere with the subsequent conjugation step with AV. In addition, the related biological materials and the conjugated complexes will also be pharmacologically safe for use in vivo.

However, in certain embodiments, the purity of the linked compounds Pr-L1 or Pr-L2 may be at least 10% on a dry matter basis to enable the selective reaction of the compounds with AV without a significant amount of interferences or without the formation of undesirable by-products obtained from the conjugate reaction with other undesired blood components. However, the desired purity of Pr, such as HSA or recombinant HSA, for example, will depend on the nature of the functional groups on Ih as well as the functional groups employed on the linker. Typically, higher purities of HSA or recombinant HSA is required if the functional groups on the linker are more reactive and may form undesired by-products than functional groups on the linker that are less reactive.

The albumin may be obtained from plasma or blood albumin from a host, purified to a desired level of purity, and linked with the linker. Purification of the albumin from blood or blood plasma may be performed using well established standard methods known in the art for the purification of albumin. Using purified blood albumin, the isolated complexes of the present will comprise of a relatively homogeneous population of functionalized proteins.

Preparation of the Complexes of Formula I or Formula II:

In one embodiment, the complexes of Formula I or Formula II may be prepared by the conjugation of AV-L1 or AV-L2 with Pr, the conjugation of Pr-L1 or Pr-L2 with AV, or the conjugation of AV with Pr to form a complex wherein the linker is absent.

In one embodiment, a solution of AV-L1 or AV-L2 is combined with Pr under conditions such that the conjugation reaction is deemed to be complete. In a particular embodiment, the linked compound is an anti-viral agent that is attached to a linker, and the linked compound is added to an aqueous solution of HSA. The resulting solution is incubated until the reaction is substantially complete.

In one embodiment, the AV-L1 or AV-L2 is combined with an excess of HSA to ensure that the conjugation reaction proceeds selectively to a single site on the HSA. For example, the formation of AV-L1 on a single site on HSA may permit ease of identification of a single complex of Formula I, for example, where n is 1. In one particular embodiment, the conjugate reaction of AV-L1 or AV-L2 with HSA occurs on a single cysteine of HSA. Without being bound by any particular theory, for some reactions, it is believed that the conjugate reaction may also occur initially with a cysteine —SH group to form a kinetic product that is then rearranged to another amino acid finctional group, such as a lysine, to form the thermodynamic product.

In another embodiment, the conjugate reaction may form the complex of Formula I, for example, wherein more than one AV is linked to a single HSA to form the complex of Formula I; that is, wherein n is greater than 1. Optionally, m may be greater than 1 if the linker L1 is a polyfunctional linker that is capable of attaching more than one AV group. In one embodiment, the complex of Formula I may be prepared by combining an excess of Pr relative to (AV)m-L1. Preferably, the ratio of Pr to (AV)m-L1 is about 50 to 100. In another particular embodiment, the ratio is from about 10 to 30. In yet another particular embodiment, the ratio is from about 2 to 5.

In one embodiment, Pr is added to (AV)m-L1 in a ratio of at least about 1.1:1, more preferably at least about 1.2:1, and most preferably at least about 1.4:1. In the case where Pr is albumin, the preferred ratios are based on the assumption that there is 0.7 free thiol per albumin. Preferably, the resulting complex is formed as a 1:1 complex, since a Pr component such as albumin has only about 70% free thiol functionality for conjugation. An excess of Pr, such as HSA or recombinant HSA is pharmacologically safe and may not require further purification. Where there is an excess of Pr in the product mixture, optionally, the conjugated complex may be purified to a purity of at least 10%. In a particular embodiment, the conjugated complex may be purified to at least about 20% or at least about 30%.

In another embodiment, the complex of Formula I may be prepared by combining an excess of (AV)m-L1 relative to Pr. Preferably, the ratio of (AV)m-L1 to Pr is about 50 to 100. In another particular embodiment, the ratio is from about 10 to 30. In yet another particular embodiment, the ratio is from about 2 to 5. Where there is an excess of (AV)m-L1 in the product mixture, optionally, the conjugated complex may be purified to a purity of at least 10%. In a particular embodiment, the conjugated complex may be purified to at least about 20% or at least about 30%.

In another embodiment, the complexes of Formula I or Formula II may be prepared from a stoichiometric ratio of (AV)m-L1 with Pr or a stoichiometric ratio of AV with L2-(Pr)o, that is, in a 1:1 ratio. Optionally, the resulting product from these preparations may be further purified to a purity of at least 10%. In a particular embodiment, the conjugated complex may be purified to at least about 20% or to a purity of at least about 30%. In yet another particular embodiment, the 1:1 conjugated complex may be further purified to a purity of greater than about 90%.

In another embodiment, the conjugated cysteine present in albumin is reduced to the free cysteine prior to the reaction.

Optionally, the complex formed from the conjugate reaction may be further purified prior to administration.

In one embodiment, the complexes of Formula I or Formula II obtained from the conjugate reaction may be administered without further processing or purification since an excess of HSA or HSA related biologically materials present with the complexes are pharmacologically safe for use in vivo.

In each of the above embodiments, AV is a peptide anti-viral agent and Pr is HSA or recombinant HSA.

In one embodiment, the isolated complex comprising a protected or unprotected anti-viral agent with a linker and albumin may be optionally further purified and then returned to the host.

The complexes formed from the methods of the present invention may be tested in animal or human hosts until the physiology, pharmacokinetics, and safety profiles are well established over an extended period of time. Typically, the measured half-life of the complexes is about 5 to 7 days, more typically at least about 7 to 10 days, and preferably 15 to 20 days or more. In general, the duration is species dependent. For example, with human albumin, the half life is about 17-19 days. Depending on the nature of the anti-viral agent, the linking group and the purity of the albumin, the effective therapeutic concentration of the complexes may be at least 1 month or more.

Half lives may be determined by serial measurements of whole blood, plasma or serum levels of the complexes of Formula I or Formula II, the AV-L compounds, the L-Pr compounds, or the AV compounds following labeling of the complex or compounds with an isotope (e.g., 131I, 125I, Tc, Cr, 3H, etc . . . ) or fluorochrome and injection of a known quantity of labeled complex or compound intravascularly. Included are red blood cells (half life ca. 60 days), platelets (half life ca. 4-7 days), endothelial cells lining the blood vasculature, and long lived blood serum proteins, such as albumin, steroid binding proteins, ferritin, α2-macroglobulin, transferrin, thyroxin binding protein, immunoglobulins, especially IgG, etc. In addition to preferred half lives, the subject components are preferably in cell count or concentration sufficient to allow binding of therapeutically useful amounts of the compound of the present invention. For cellular long lived blood components, cell counts of at least 2,000/μl and serum protein concentrations of at least 1 μg/ml, usually at least about 0.01 mg/ml, more usually at least about 1 mg/ml, are preferred.

However, where the nature of the complex is designed such that the biologically active agent AV, such as an anti-viral agent, is to be cleaved from the complex and released into the host, the desired half life for the effective therapeutic concentration of the complex and/or the biologically active agent may vary from the measured half-life above. The rate of release of the biologically active agent depends in part, on the valency or the functionality on the biological agent which is to be released, the nature of the linking group, the purity and type of the protein, the composition for administration, the manner of administration, and the like. Thus, various linking groups and biological agents may be employed, where the environment of the blood, components of the blood, particularly enzymes, activity in the liver, or other agent may result in the cleavage of the linking group with release of the biological agent in the host at a desired rate.

The isolated complexes of the present invention provides biological active compounds that have improved pharmacokinetics, solubility, bioavailability, distribution, and/or immunogenicity characteristics as compared to the non-conjugated compounds.

Surprisingly, the complexes of Formula I and Formula II, when prepared and used according to the methods of the present invention, provides an effective therapeutic concentration for a significantly longer time than the AV component by itself. In addition, the complexes of the present invention provide improved solubility, distribution, pharmacokinetics, and result in decrease immunogenicity when compared to the administration of the AV component by itself.

The present inventors surprisingly have found that administration to a subject of a conjugate that is prepared ex vivo from purified components (specifically HSA, linker and an anti-viral agent) produces a remarkably efficient tissue vivo distribution of the conjugate compared to conjugates that are prepared by in vivo preparation of the conjugate by injection of an activated compound that binds in situ to endogenous albumin in the blood stream of the subject. Moreover, the present inventors have found that substantially all of the conjugate remains in circulation for hours or even days following administration, compared to the dramatic losses of compound that are observed when the conjugate is prepared in vivo. This efficiency reduces the number of times that the patient must be subjected to injection of active substance, and also reduces the amount of anti-viral agent that must be given in a single administration.

In the context of the present invention, a therapeutically effective amount of a composition is understood to mean an amount that, when administered to a subject, produces a desired physiological effect to a degree that is effective for treatment of a disease, condition, or syndrome in the patient, or that is effective in alleviating the symptoms disease, condition, or syndrome.

For preparation of fusion proteins containing the blood component and a peptide anti-viral, the genes encoding the fusion protein are placed into a suitable vector in frame, and the vector is used to transform a suitable host cell. The genes may be placed in either order (i.e. the anti-viral peptide may be placed at the N- or C-terminus of the fusion protein) and may be directly fused or separated by a linker peptide. Suitable linker peptides are known in the art and include peptide sequences that have little secondary structure of their own and that are hydrophilic, for example, linkers containing mixtures of glycine and serine residues. Methods for making fusion proteins of HSA are described in WO01/79271 and WO01/9258, and similar methods can be used for making fusion proteins with other blood components.

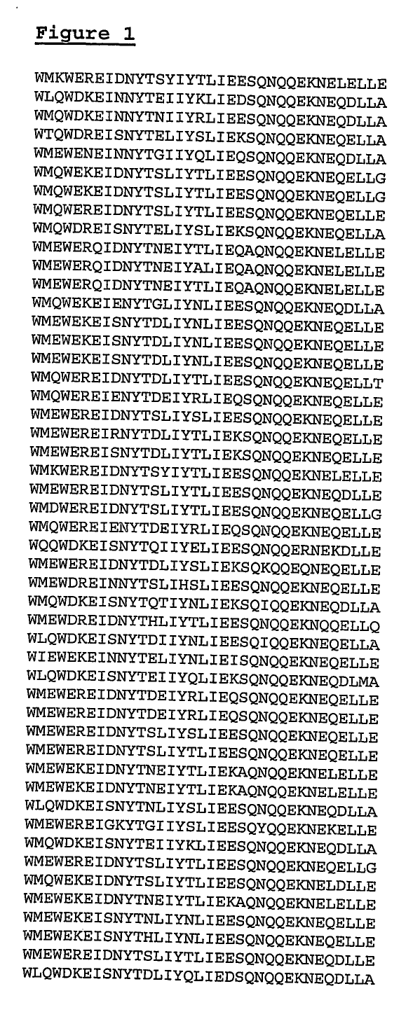

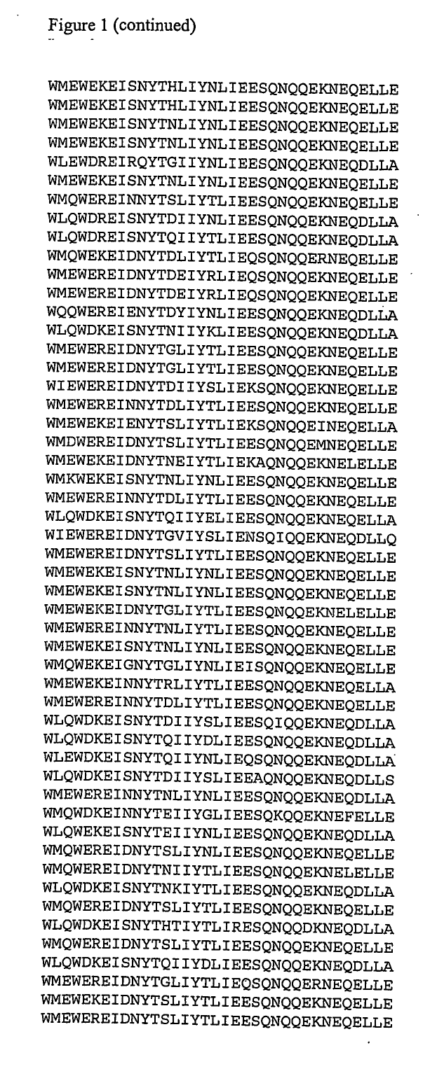

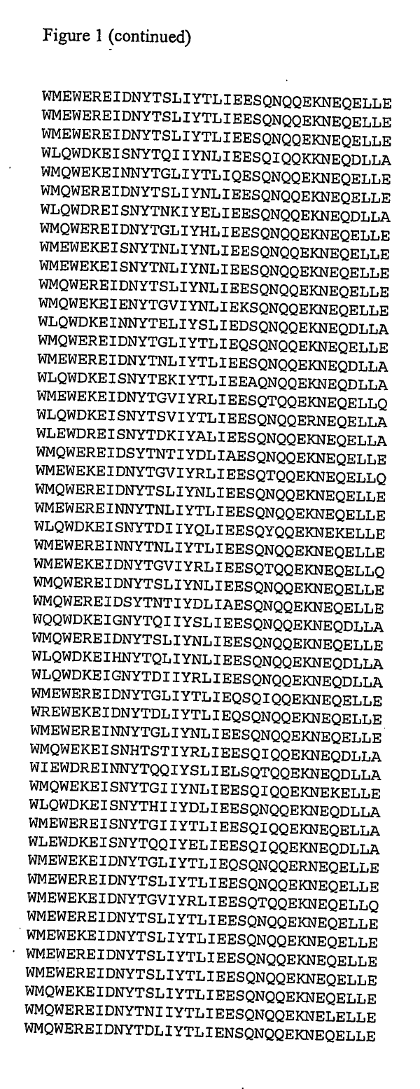

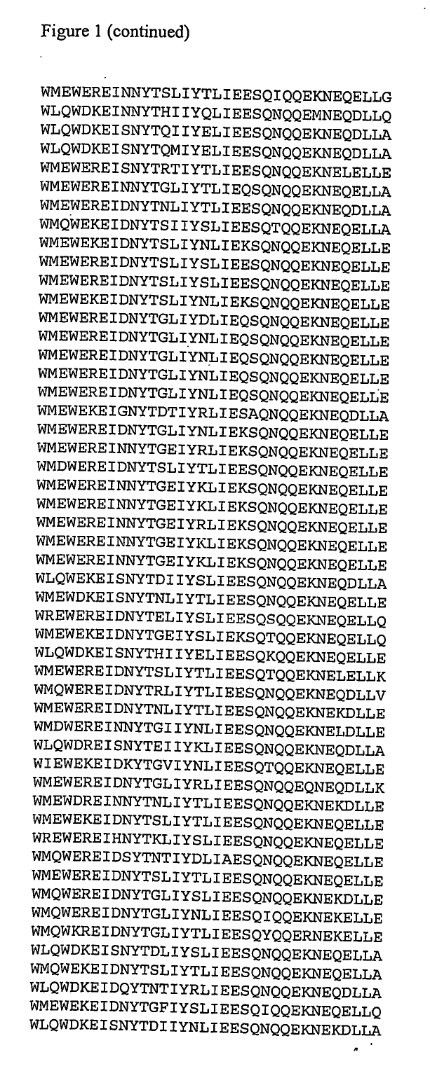

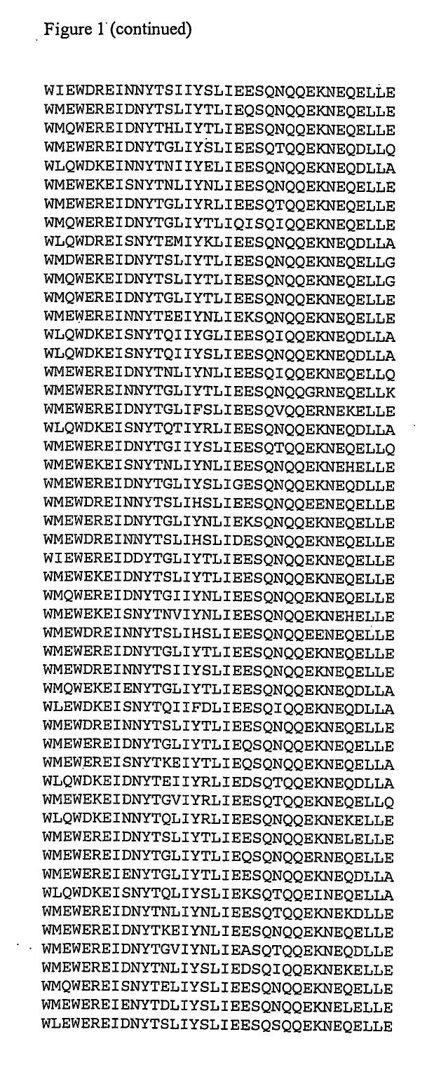

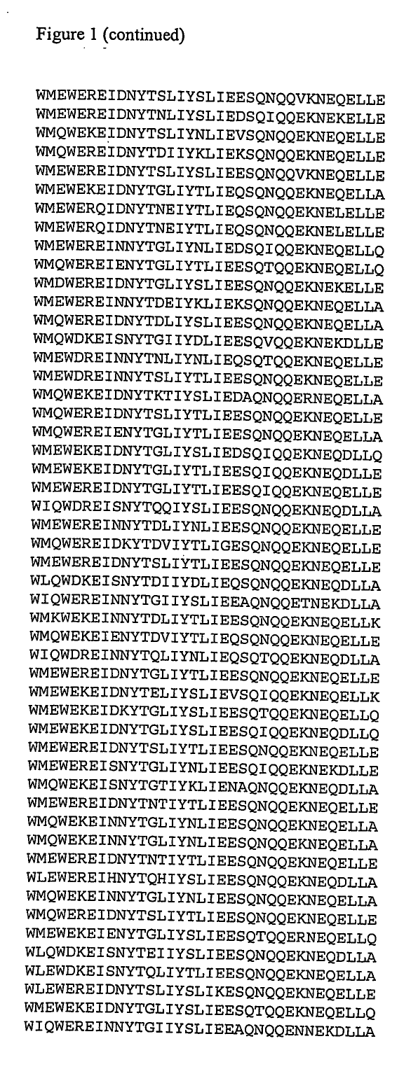

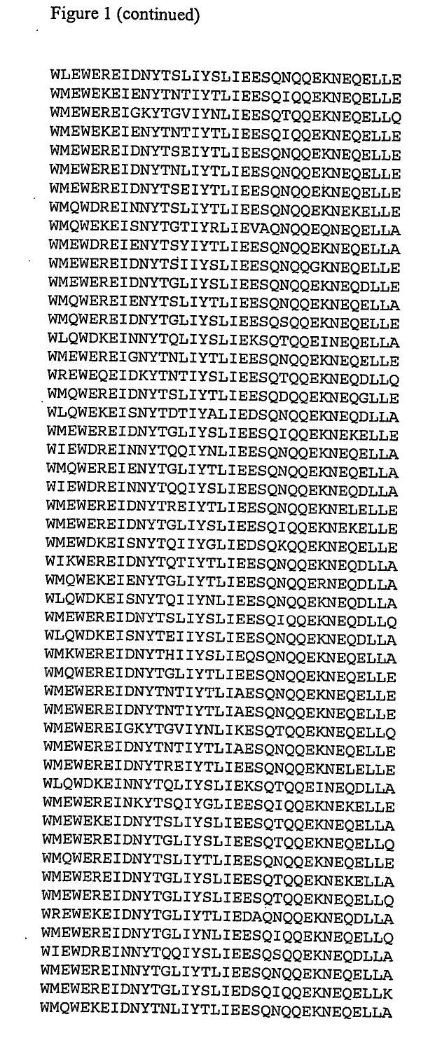

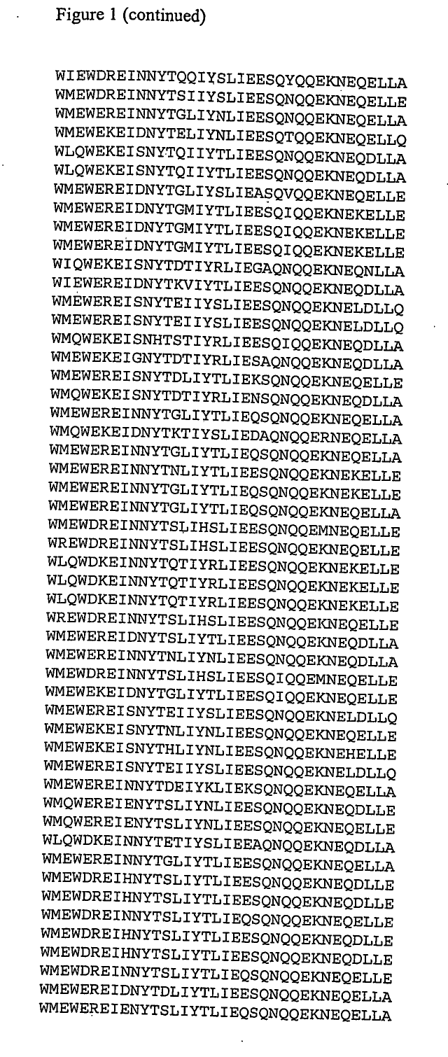

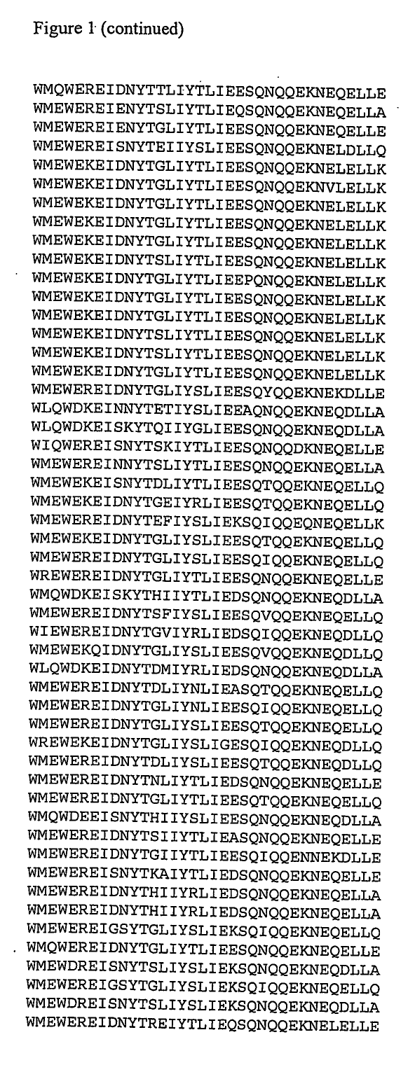

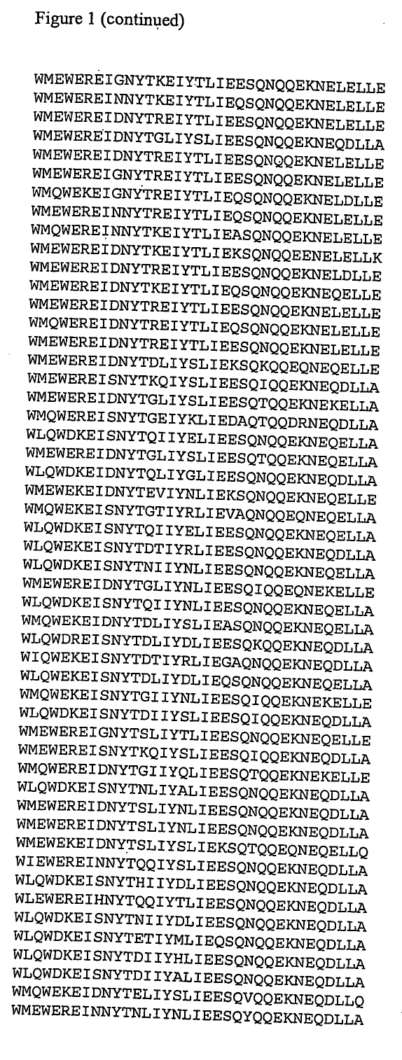

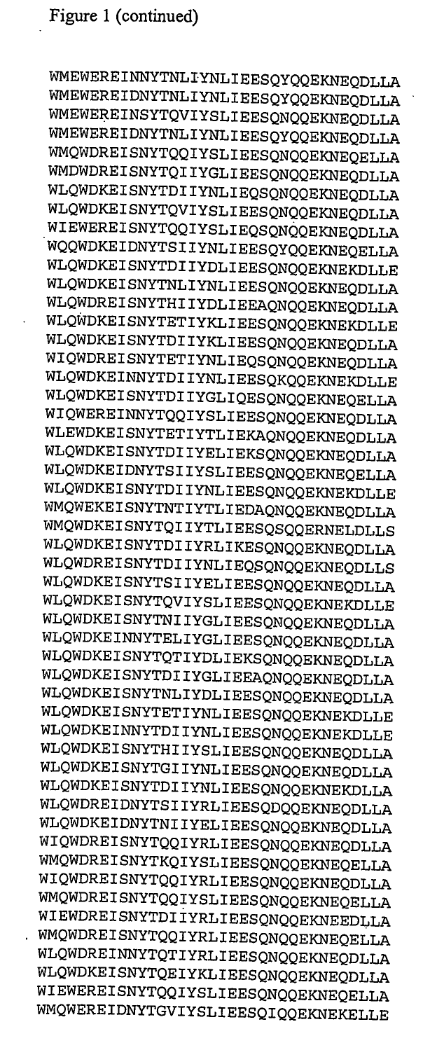

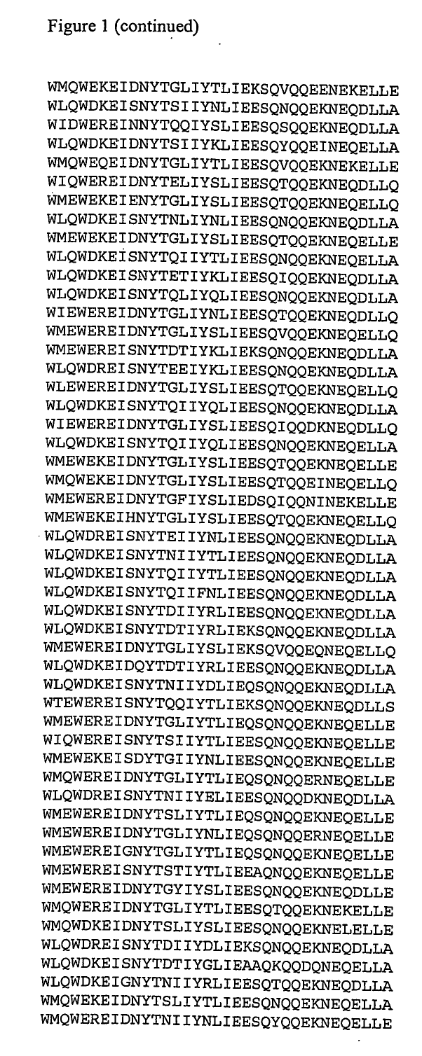

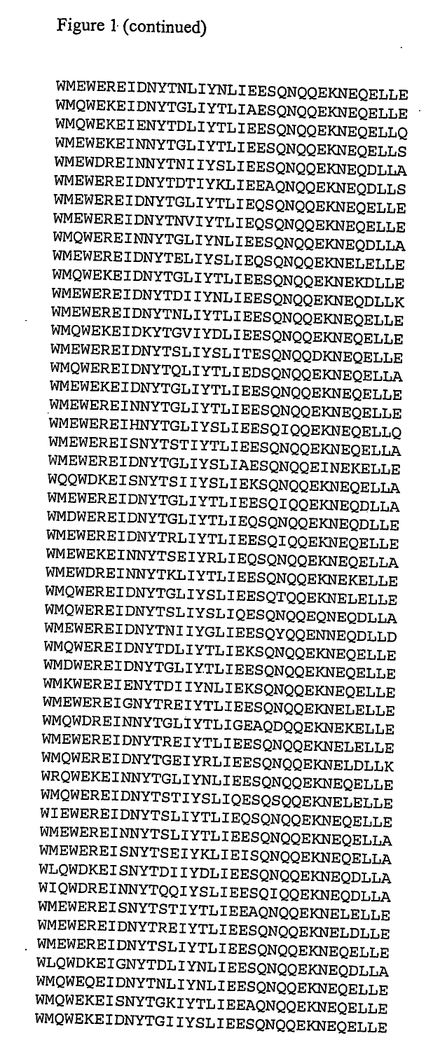

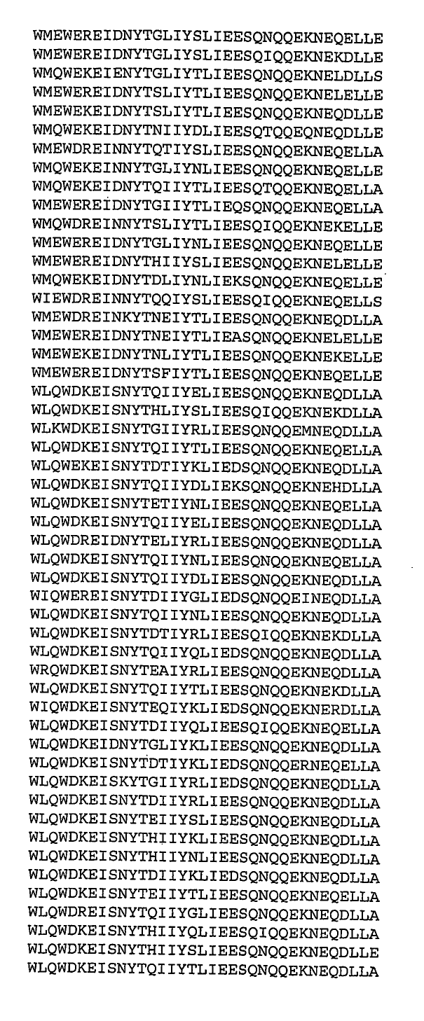

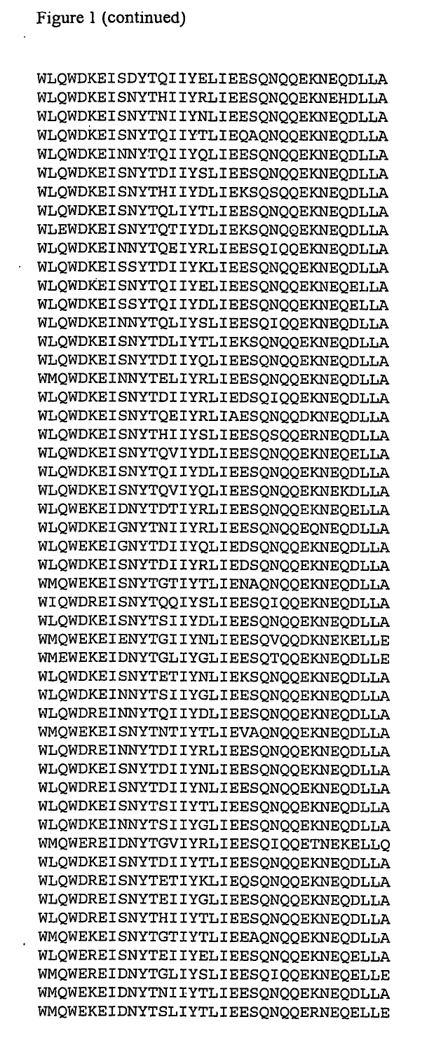

The present invention particularly contemplates use of peptides that inhibit viral fuision with the cell membrane, and in particular contemplates peptides that inhibit fusion of HIV. Specific peptide inhibitors typically contain up to about 51 amino acids, and contains a peptide having the sequences as discosed herein. In particular, the peptides may contain the sequences shown in FIG. 1. These sequences are derived from sequences found in EHIV isolates and, for peptides longer than the sequences shown in FIG. 1, for example, the remainder of the peptide sequence can be either N- or C- terminal to the sequence shown. Such additional sequences can, for example, consist of, or can contain, the sequences that occur adjacent to the defined sequences in those HIV isolates.

Administration of the Isolated Complexes of Formula I and Formula II:

In one embodiment, the administration of the isolated complex of the present invention may be accomplished using a bolus, but may be introduced slowly over time by transfusion using metered flow, or the like.

The complex of the present invention may be administered in a physiologically acceptable medium, e.g. deionized water, phosphate buffered saline, saline, mannitol, aqueous glucose, alcohol, vegetable oil, or the like. A single injection may be employed although more than one injection may be used, if desired. The complex may be administered by any convenient means, including syringe, trocar, catheter, or the like. The particular manner of administration, will vary depending upon the amount to be administered, whether a single bolus or continuous administration, or the like. The administration may be intravascularly, where the site of introduction is not critical to this invention, preferably at a site where there is rapid blood flow, e.g. intravenously, peripheral or central vein. Other routes may find use where the administration is coupled with slow release techniques or a protective matrix.

Surprisingly, it is noted that the administration of the isolated complexes prepared by the methods of the present invention, for example, from isolated blood protein, such as albumin, results in anti-viral conjugate complexes that maintain an effective therapeutic effect in the bloodstream for an extended period of time as compared to a non-conjugated anti-viral agents or as compared to complexes that are not prepared from isolated blood protein such as albumin.

In one embodiment, the present invention provides the compounds in the form of a pharmaceutically acceptable salt.

In another embodiment, the present invention provides the compounds present in a mixture of stereoisomers. In yet another embodiment, the present invention provides the compounds as a single stereoisomer.

In yet another embodiment, the present invention provides pharmaceutical compositions comprising the compound as an active ingredient. In yet another particular variation, the present invention provides pharmaceutical composition wherein the composition is a tablet or a solid for administration as a depot. In another particular variation, the present invention provides the pharmaceutical composition wherein the composition is a liquid formulation adapted for IV or subcutaneous administration. In yet another particular variation, the present invention provides pharmaceutical composition wherein the composition is a liquid formulation adapted for parenteral administration.

It is noted in regard to all of the embodiments, and any further embodiments, variations, or individual compounds described or claimed herein that all such embodiments, variations, and/or individual compounds are intended to encompass all pharmaceutically acceptable salt forms whether in the form of a single stereoisomer or mixture of stereoisomers unless it is specifically specified otherwise. Similarly, when one or more potentially chiral centers are present in any of the embodiments, variations, and/or individual compounds specified or claimed herein, both possible chiral centers are intended to be encompassed unless it is specifically specified otherwise.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al.(1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3rd edition, John Wiley & Sons, Inc. 1999.

Compounds of the present invention may also be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxane, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which is utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Peptides and Complexes:

In one embodiment of the invention, there is provided a peptide consisting of up to 51 amino acids comprising the sequence

Y1-X—X—Y2-X—X—X—Y3-X—X—X—Y4-X—X—Y5-X—X—Y6, wherein:

the sequence is located at the N-terninal, C-terminal or at an interior position of the peptide;

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of L V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C; and,

each X independently is any amino acid.

In another embodiment of the invention, there is provided a peptide consisting of up to 51 amino acids comprising the sequence

Y1-X—X—Y2-X—X—X—Y3-X—X—X—Y4-X—X—Y5-X—X—Y6-Y7, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q. K, R, H, E and D; and

each X independently is any amino acid.

In another embodiment of the invention, there is provided a peptide consisting of up to 51 amino acids comprising the sequence

Y1-X—X—Y2-X—X—X—Y3-X—X—X—Y4-X—X—Y5-X—X—Y6-Y7-X—X—X—Y8, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of L V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R,N, E, D, K and P; and

each X independently is any amino acid.

In another embodiment of the invention, there is provided a peptide consisting of up to 51 amino acids comprising the sequence

Y1-X—X—Y2-X—X—X—Y3-X—X—X—Y4-X—X—Y5-X—X—Y6-Y7-X—X—X—Y8-X—Y9, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P; and

each X independently is any amino acid.

In another embodiment of the invention, there is provided a peptide consisting of up to 51 amino acids comprising the sequence

Y1-X—X—Y2-X—X—X—Y3-X—X—X—Y4-X—X—Y5-X—X—Y6-Y7-X—X—X—Y8-X—Y9-Y10, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R. Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R; and

each X independently is any amino acid.

In another embodiment of the invention, there is provided a peptide of up to 51 amino acids comprising the sequence

Y1-X—X—Y2-X—X—X—Y3-X—X—X—Y4-X—X—Y5-X—X—Y6-Y7-X—X—X—Y8-X—Y9-Y10-X—X—Y11, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and is C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D; and

each X independently is any amino acid.

In another embodiment of the invention, there is provided a peptide of up to 51 amino acids comprising the sequence

Y1-X—X—Y2-X—X—X—Y3-X—X—X—Y4-X—X—Y5-X—X—Y6-Y7-X—X—X—Y8-X—Y9-Y10-X—X—Y11-Y12, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S; and

each X independently is any amino acid.

In another embodiment of the invention, there is provided a peptide of up to 51 amino acids comprising the sequence

Y1-X—X—Y2-X—X—X—Y3-X—X—Y4-X—X—Y5-X—X—Y6-Y7-X—X—X—Y8-X—Y9-Y10-X—X—Y11-Y12-X—X—Y13, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of L V, L, A, S and T;

Y4 is selected from the group consisting of T, S, L K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of L L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y1 I is selected from the group consisting of N, S, T, V, A and D;

Y12 is selected from the group consisting of E, V, K, G, R, Q, D, N, H, T and S;

Y13 is selected from the group consisting of L, I, V, K and R; and

each X independently is any amino acid.

In another embodiment of the invention, there is provided a peptide of up to 51 amino acids comprising the sequence

Y1-X—X—Y2-X—X—X—Y3-X—X—X—Y4-X—X—Y5-X—X—Y6-Y7-X—X—X—Y8-X—Y9-Y10-X—X—Y11-Y12-X—X—Y13-Y14, wherein

Y1 is selected from the group consisting of W, Y, F, H, L, N, Q, E, D, K, and R;

Y2 is selected from the group consisting of W, Y, F, H, L, N, Q, E, D, K, and R;

Y3 is selected from the group consisting of I, V, L, A, S and T;

Y4 is selected from the group consisting of T, S. I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide consisting of up to 51 amino acids comprising the sequence

Y2-X—X—X—Y3-X—X—X—Y4-X—X—Y5-X—X—Y6-Y7-X—X—X—Y8-X—Y9-Y10-X—X—Y11-Y12-X—X—Y13-Y14, wherein:

Y2 is selected from the group consisting of W, Y, F, H, L, N, Q, E, D, K, and R;

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide of up to 51 amino acids comprising the sequence

Y3-X—X—X—Y4-X—X—Y5-X—X—Y6-Y7-X—X—X—Y8-X—Y9-Y10-X—X—Y11-Y12-X—X—Y13-Y14, wherein:

Y3 is selected from the group consisting of I, V, L, A, S and T;

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide consisting of up to 51 amino acids comprising the sequence

Y4-X—X—Y5-X—X—Y6-Y7-X—X—X—Y8-X—Y9-Y10-X—X—Y11-Y12-X—X—Y13-Y14, wherein:

Y4 is selected from the group consisting of T, S, I, K, N, H, R, Q, E and D;

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide consisting of up to 51 amino acids comprising the sequence

Y5-X—X—Y6-Y7-X—X—X—Y8-X—Y9-Y10-X—X—Y11-Y12-X—X—Y13-Y14, wherein:

Y5 is selected from the group consisting of I, V, T, K, L, N, Q, D, E, R and H;

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T is and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide consisting of up to 51 amino acids comprising the sequence

Y6-Y7-X—X—X—Y8-X—Y9-Y10-X—X—Y11-Y12-X—X—Y13-Y14, wherein:

Y6 is selected from the group consisting of any amino acid except P, G and C;

Y7 is selected from the group consisting of I, L, V, N, Q, K, R, H, E and D;

Y8 is selected from the group consisting of Q, H, R, N, E, D, K and P;

Y9 is selected from the group consisting of Q, H, N, E, D, K, R, L and P;

Y10 is selected from the group consisting of Q, H, N, E, D, K and R;

Y11 is selected from the group consisting of N, S, T, V, A and D;

Y12 is selected from the group consisting of E, V, K, G, R, Q, D, N, H, T and S;

Y13 is selected from the group consisting of L, I, V, K and R;

Y14 is selected from the group consisting of L, S, M, Y, N, Q, E, D, K, and R; and

each X independently is any amino acid.

In another embodiment of the invention, there is provided a peptide consisting of up to 51 amino acids comprising the sequence

W—X—X—W—X—X—X—I—X—X—X-T-X—X—I—X—X-L-I—X—X—X-Q-X-Q-Q-X—X—N, wherein:

each X independently is any amino acid.

In another embodiment of the invention, there is provided a peptide consisting of up to 51 amino acids comprising the sequence

W—X1-X2-W—X3-X4-X5-I—X6-X7-X8-T-X9-X10-I—X11-X12-L-I—X13-X14-X15-Q-X16-Q-Q-X17-X18-N-X19-X20-X21-X22-X23, wherein:

X1 is selected from the group consisting of M, L, I, Q, T, R and K;

X2 is either E, D, Q and K;

X3 is selected from the group consisting of E, D and K;

X4 is selected from the group consisting of K, R, E, Q, N and T;

X5 is selected from the group consisting of E, L, R, K and Q;

X6 is selected from the group consisting of N, D, S, E, Q, K, R, H, T, I and G;

X7 is selected from the group consisting of N, Q, D, E, K, S, T and Y;

X8 is selected from the group consisting of Y, F, H, I, V and S;

X9 is selected from the group consisting of G, K, R, H, D, E, S, T, N and Q;

X10 is selected from the group consisting of K, H, E, Q , T, V, I, L, M, A, Y, F, and P;

X11 is selected from the group consisting of H, K, E, Y and F;

X12 is selected from the group consisting of T, S, Q, N, E, D, R, K, H, W, G, A, and M;

X13 is selected from the group consisting of D, E, Q, T, K, R, A, V and G;

X14 is selected from the group consisting of D, E, K, H, Q, N, S, I, L, V, A and G;

X15 is selected from the group consisting of S, A and (P);

X16 is selected from the group consisting of N, K, S, T, D, E, Y, I and V;

X17 is selected from the group consisting of E, D, N, K, G, and V;

X18 is selected from the group consisting of K, R, H, D, E, N, Q, T, M, I, and Y;

X19 is selected from the group consisting of E, V, Q, M, L, J, and G;

X20 is selected from the group consisting of Q, N, E, K, R, H, L, and F;

X21 is selected from the group consisting of E, D, N, S, K, A, and G;

X22 is selected from the group consisting of L, I, and Y; and

X23 is selected from the group consisting of I, L, M, Q, S, and Y.

In one variation of the above embodiment, the peptide comprises a sequence selected from the group consisting of the sequences shown in FIG. 1.

In another embodiment of the invention, there is provided an isolated complex of the Formula I or Formula II:

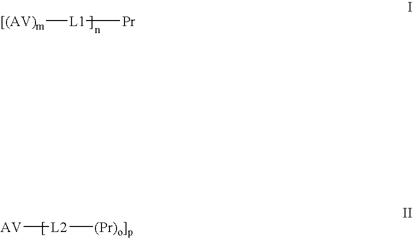

wherein:

m is an integer from 1-5;

n is an integer from 1-100;

o is an integer from 1-5;

p is an integer from 1-100;

AV is an antiviral compound;

L1 and L2 are polyvalent linkers covalently linking AV to Pr, or where L1 and L2 are absent;

Pr is a protein; and

wherein the complex possesses antiviral activity in vivo.

In one variation of the above embodiment, the antiviral compound is a peptide. In another variation, the peptide has a mass of less than about 100 kDA.

In another variation, the peptide has a mass of less than about 30 kDA. In yet another variation, the peptide has a mass of less than about 10 kDA.

In one particular variation, the peptide is a peptidomimetic.

In another embodiment of the invention, the peptide consists of up to 51 amino acids comprising a sequence selected from the group consisting of:

Y1-X-X-Y2-X-X-X-Y3-X-X-X-Y4-X-X-Y5-X-X-Y6;
Y1-X-X-Y2X-X-X-Y3-X-X-X-Y4-X-X-Y5-X-X-Y-X-Y6-Y7;
Y1-X-X-Y2-X-X-X-Y3-X-X-X-Y4X-X-Y5X-X-Y6-Y7-X-X-X-
Y8-X-Y9;
Y1-X-X-Y2-X-X-X-Y3-X-X-X-Y4X-X-Y5X-X-Y6-Y7-X-X-X-
Y8-X-Y9-Y10;
Y1-X-X-Y2-X-X-X-Y3-X-X-X-Y4-X-X-Y5-X-X-Y6-Y7-X-X-
X-Y8-X-Y9-Y10-X-X-Y11;
Y1-X-X-Y2-X-X-X-Y3-X-X-X-Y4-X-X-Y5-X-X-Y6-Y7-X-X-
X-Y8-X-Y9-Y10-X-X-Y11-Y12;
Y1-X-X-Y2-X-X-X-Y3-X-X-X-Y4-X-X-Y5-X-X-Y6-Y7-X-X-
X-Y8-X-Y9-Y10-X-X-Y11-Y12-X-X-Y13;
Y1-X-X-Y2-X-X-X-Y3-X-X-X-Y4-X-X-Y5-X-X-Y6-Y7-X-X-
X-Y-X-Y9-Y10-X-X-Y11-Y12-X-X-Y13-Y14;
Y2-X-X-X-Y3-X-X-X-Y4-X-X-Y5-X-X-Y6-Y7-X-X-X-Y8-X-
Y9-Y10-X-X-Y11-Y12-X-X-Y13-Y14;
Y3-X-X-X-Y4-X-X-Y5-X-X-Y6-Y7-X-X-X-Y8-X-Y9-Y10-X-
X-Y11-Y12-X-X-Y13-Y14;
Y4-X-X-Y5-X-X-Y6-Y7-X-X-X-Y8-X-Y9-Y10-X-X-Y11-Y12-
X-X-Y-13-Y14;
Y5-X-X-Y6-Y7-X-X-X-Y8-X-Y9-Y10-X-X-Y11-Y12-X-X-
Y13-Y14;
Y6-Y7-X-X-X-Y8-X-Y9-Y10-X-X-Y11-Y12-X-X-Y13-Y14;
W-X-X-W-X-X-X-I-X-X-X-T-X-X-I-X-X-L-I-X-X-X-Q-X-Q-
Q-X-X-N;
W-X1-X2-W-X3-X4-X5-I-X6-X7-X8-T-X9-X10-I-X11-X12-
L-I-X13-X14-X15-Q-X16-Q-Q-X17-X18-N-X19-X20-X21-
X22-X23;

peptide DP178 (T-20); and

peptide T-1249;

wherein:

X1 is selected from the group consisting of M, L, I, Q, T, R and K;

X2 is either E, D, Q and K;

X3 is selected from the group consisting of E, D and K;

X4 is selected from the group consisting of K, R, E, Q, N and T;

X5 is selected from the group consisting of E, L, R, K and Q;

X6 is selected from the group consisting of N, D, S, E, Q, K, R, H, T, I and G;

X7 is selected from the group consisting of N, Q, D, E, K, S, T and Y;

X8 is selected from the group consisting of Y, F, H, I, V and S;

X9 is selected from the group consisting of G, K, R, H, D, E, S, T, N and Q;

X10 is selected from the group consisting of K, H, E, Q, T, V, I, L, M, A, Y, F, and P;

X11 is selected from the group consisting of H, K, E, Y and F;

X12 is selected from the group consisting of T, S, Q, N, E, D, R, K, H, W, G, A, and M;

X13 is selected from the group consisting of D, E, Q, T, K, R, A, V and G;

X14 is selected from the group consisting of D, E, K, H, Q, N, S, I, L, V, A and G;

X15 is selected from the group consisting of S, A and (P);

X16 is selected from the group consisting of N, K, S, T, D, E, Y, I and V;

X17 is selected from the group consisting of E, D, N, K, G, and V;

X18 is selected from the group consisting of K, R, H, D, E, N, Q, T, M, I, and Y;

X19 is selected from the group consisting of E, V, Q, M, L, J, and G;

X20 is selected from the group consisting of Q, N, E, K, R, H, L, and F;

X21 is selected from the group consisting of E, D, N, S, K, A, and G;

X22 is selected from the group consisting of L, I, and Y; and

X23 is selected from the group consisting of I, L, M, Q, S, and Y.

The peptide sequence disclosed herein comprising the fragment of the peptide that consists of up to 51 amino acids may be a fragment of the 51 amino acid peptide that is located at the N-terminus, the C-terminus or anywhere in the interior of the 51 amino acid peptide. In one variation, the peptides are the C-termninus amides (—CONH₂) and their protected derivatives. In another variation, the peptides are the C-terminus esters (i.e. —COOR, where R is substituted or unsubstituted (C₁₋₁₅)alkyls).

Optionally, the peptide sequence disclosed herein comprising the fragment may be further protected by standard protecting groups known in the art. Protected derivatives of these peptides are useful in the preparation of the antiviral compounds or are useful in themselves as active antiviral compounds in their partially or fully protected forms. That is, the derivatized or protected or partially protected peptide fragments in the complex may still retain the ability to bind the target and manifest therapeutic biological activities. For example, representative protecting groups for amino groups of the peptide fragments include acetyl, tert-butoxycarbonyl, benzyloxycarbonyl, and the like. Suitable and representative protecting groups can be found in T. W. Greene, Protecting Grouips in Organic Synthesis, 3rd edition, John Wiley & Sons, Inc. 1999.

In one embodiment, the protecting group for the peptide fragments comprise C-terminal amides and/or N-terminal acetyl groups and their derivatives. The peptides may have any functional groups of the amino acids, including —NH, —SH, —OH, —COOH, and the like, that may be attached to the linker.

In one variation of the invention, there is provided a complex of the above embodiments and variations wherein the protein is a blood component. In another variation, the blood component is selected from the group consisting of red blood cells, immunoglobulins, IgM, IhG, serum albumin, transferrin, P90 and P38, ferritin, a steroid binding protein, thyroxin binding protein, and α-2-macroglobulin. In yet another variation, the blood component is human serum albumin and the linker is a peptide linker.

In one particular variation, the blood component is human serum albumin and the linker is a non-peptide linker.

In one particular embodiment of the invention, the complex is a fusion protein.

In one variation of the invention, the linler L1 or L2 is a non-labile linker that is stable toward hydrolytic cleavage in vivo. Therefore, the complexes of the present invention provides compounds that are stable toward hydrolytic cleavage in vivo. In addition, the complexes of the present invention are also active compounds in themselves and are not simply a prodrug of the peptides that are generated or released upon hydrolysis in vivo.

WO 00/76550 (F. Kratz) discloses pharmaceuticals and/or diagnostic active substances attached to a spacer molecule that is attached to a thiol binding group, such as native or recombinant albumin. However, the disclosure teaches that the release of the pharmaceutical compounds or the diagnostic active substances is prefered since the low molecular weight active substance must interact with the target molecule to that it is pharmacologically active. In addition, Kratz teaches that the spacer molecule are selected from compounds that are hydrolytically and/or pH-dependent and/or are ezymatically scissile. Preferably, these spacer molecules are acid sensitive or acid-unstable spacers.

The linker L1 or L2 can be a hydrophobic linker, a hydrophilic linker, or combinations thereof when more than one linker is present. The variety of different linker L1 or L2 can be selected to provide different solubility characteristics and cell penetrability chracteristics.

Where the antiviral compound of the present invention is a peptide that is attached to one or more linkers, the linker L1 or L2 may be attached to the peptide at the N-terminus, the C-teminus, at a reactive side chain on an internal amino acid(s) such as, for example, with a lysine, aspartic acid, glutamic acid, or cysteine, or combinations thereof.

In one variation of the invention, the linker L1 or L2 comprises at least two functional groups covalently linking AV to Pr. In another variation, the linker L1 or L2 is hydrolytically stable in human serum for an extended period of time.

It was determined that the complexes of the present invention are compounds that are themselves more stable toward hydrolytic cleavage or degradation than the non-complexed compounds. In one variation, the complex of the present invention are stable toward hydrolytic cleavage or degradation, having half lives in human serum for a period of 4 hours to 120 days. In a particular variation, the complex of the present invention are stable toward hydrolytic cleavage or degradation for a period of about 8 hours to about 30 days.

In one particular variation, there is provided the complexes of the invention wherein the linker L1 or L2 is stable in human serum for half lives of 8 hours to 30 days.

In another particular variation of the above variations and embodiments, the linker L1 or L2 is a derivative of a compound selected from the group consisting of acyloxymethylketones, aziridines, diazomethyl ketones, epoxides, iodo-, bromo- or chloroacetamides, α-haloesters, α-haloketones, sulfoniums, chloroethylsulfides, O-alkylisoureas, alkyl halides, vinylsulfones, acrylamides, acrylates, vinylpyridines, organometallic compounds, aryldisulfides, thiosulfonates, aldehydes, nitriles, α-diketones, α-ketoamides, α-ketoesters, diaminoketones, semicarbazones, and dihydrazides.

In another particular variation of the above variations and embodiments, the linker L1 or L2 is a derivative of a compound selected from the group consisting of azidobenzoyl hydrazide, N-[4-(p-azidosalicylamino)butyl]-3′-(2′-pyridyldithio)propionamide, bis-sulfosuccinimidyl suberate, dimethyl adipimidate, disuccinimidyl tartrate, N-y-maleimidobutyryloxysuccinimide ester, N-hydroxy sulfosuccinimidyl-4-azidobenzoate, N-succinimidyl[4-azidophenyl]-1,3′-dithiopropionate, N-succinimidyl [4-iodoacetyl]aminobenzoate, glutaraldehyde, succinimidyl 4-[N-maleimidomethyl]cyclohexane-1-carboxylate, N-hydroxysulfosuccinimide, maleimide-benzoyl-succinimide, γ-maleimido-butyryloxy succinimide ester, maleimidopropionic acid, N-hydroxysuccinimide, isocyanate, thioester, thionocarboxylic acid ester, imino ester, carbodiimide, anhydride and carbonate ester.

In one particular embodiment, there is provided a complex of the invention wherein the protein is albumin. In one variation of the above embodiment, the albumin is HSA or recombinant HSA that is at least 10% pure on a dry matter basis.

In another variation, the linkage is to a Cys-34 of human albumin. In yet another variation, the linkage is to a lysine of human albumin.

In one particular variation of the invention, there is provided the above disclosed complex wherein m is 1, n is 1, and the protein is HSA or recombinant HSA. In another variation, n is 1, the protein is HSA or recombinant HSA, and wherein the complex is further purified to a purity of at least 30%. In yet another particular variation, m is 1, n is 2, and the protein is HSA or recombinant HSA.

In one embodiment, the complex is prepared by combining a stoichiometric ratio of (AV)ₘ-L1 with Pr or a stoichiometric ratio of AV with L2-(Pr)ₒ. Thus, the ratio of (AV)ₘ-L1 to Pr, or the ratio of AV to L2-(Pr)ₒ are 1:1. In another particular variation, the complex is prepared by combining a mixture of Pr to (AV)ₘ-L1 in a ratio of at least about 1.3:1.

In one variation of the above embodiment, L1 and L2 are absent, and wherein the complex is prepared by forming an activated intermediate of AV followed by the condensation of the activated AV intermediate with Pr. In a variation of the above, the activated intermediate of AV is prepared from a mixed anhydride or N,N′-carbonyldiimidazole reagent.

According to the above variations, the complex is further purified to a purity of at least about 30%. Unexpectedly, it was determined that the formulation of the conjugate compound ex vivo produces unanticipated advantages over forming of the conjugated compound in vivo. For example, in the case of the relatively insoluble antiviral agents, conjugation ex vivo forms a more soluble agent or complex. In addition to improved the stability of the compounds, the formation of the complex of the present invention result in a more soluble complex for formulation, which is a significant advantage for the administration (via injection) over the administration of the insoluble unconjugated drug. Because of the ex vivo conjugation of the antiviral agent forms a soluble drug formulation, the present method allows the preparation of stable, physiological solutions. The ability to prepare stable, soluble solution compositions containing the complex of the present invention allows the preparation of physiological saline solution of the complex for ease of oral or parenteral administration.

In addition, the formation of the complex ex vivo has been found to be preferable over the in vivo formation of the complex because the administration of the complex results in less irritation at the injection site, avoids the non-specific reaction with other proteins, and achieves an improved therapeutic blood levels of the complex than the in vivo approach.

In another embodiment, the invention provides an anti-viral composition comprising a non-peptidic anti-viral compound covalently linked to a blood component.

According to each of the above embodiments and variations, there is provided according to the above embodiments and variations a composition comprising the complex and a physiologically acceptable carrier. In one variation, the composition is formulated with saline or formulated without saline. In another variation, the composition is formulated for parenteral administration.

Administration of the composition of the present invention may include parenteral administration, including by injection through other route such as subcutaneous, intramuscular, intraorbital, intracapsular, intraspinal, intrasternal, intracerebral ventricular (ICV), intravenous, and the like.

In one variation of the above, the composition is selected from the group consisting of solutions, dry products for combining with a solvent prior to use, suspensions, emulsions, and liquid concentrates.

In another embodiment of the invention, there is provided a method for inhibiting the activity of HIV gp41 and HIV in vivo, the method comprising:

administering to the bloodstream of a mammalian host an isolated conjugate complex of the above embodiments and variations, wherein the complex is formed by attaching an antiviral compound to a linker having at least one reactive functional group which reacts with the protein to form stable covalent bonds; and

wherein the isolated conjugate complex is administered in an amount to maintain an effective therapeutic effect in the bloodstream for an extended period of time as compared to a non-conjugated antiviral compound.

In one variation of the above method, the method may be applicable to the complexes disclosed in the above embodiments and variations.

In another variation, the method employs a protein wherein the protein is HSA or recombinant HSA.

In a particular variation of the above method, the linker comprising a reactive functional group is a compound selected from the group consisting of acyloxymethylketones, aziridines, diazomethyl ketones, epoxides, iodo-, bromo- or chloroacetamides, α-haloesters, α-haloketones, sulfoniums, chloroethylsulfides, O-alkylisoureas, alkyl halides, vinylsulfones, acrylamides, acrylates, vinylpyridines, organometallic compounds, aryldisulfides, thiosulfonates, aldehydes, nitriles, α-diketones, α-ketoamides, α-ketoesters, diaminoketones, semicarbazones, and dihydrazides.

In one embodiment, there is provided a method for eliciting antiviral activity in vivo, said method comprising:

administering into the bloodstream of a mammalian host the complex of the above embodiments and variations in an amount sufficient to provide an effective amount for antiviral activity;

whereby said complex is maintained in the bloodstream over an extended period of time as compared to the lifetime of unbound antiviral compound.

In another embodiment of the invention, there is provided a method for eliciting antiviral activity in a host, said method comprising:

a) preparing a compound AV-L1 or AV-L2 wherein AV is a peptide antiviral compound with a mass of less than 60 kD and L1 or L2 is a linker covalently bound to AV;

b) treating the compound AV-L1 or AV-L2 with isolated protein ex vivo for a time sufficient for the compound AV-L1 or AV-L2 to covalently bond to the protein to form the protein complex of the above embodiments and variations, and

c) administering the treated protein complex to the host.

In one variation of the above embodiment, the protein is albumin. In another variation of the above, the albumin is HSA or recombinant HSA.

According to one variation, the albumin is obtained from blood, purified and isolated from blood prior to treating the albumin with the compound AV-L1 or AV-L2. In another variation of the above methods, the albumin is purified to a purity level of at least 10% on a dry matter basis. In yet another variation, the albumin is purified to a purity level of more than 95%.

In another embodiment, the invention provides a method for eliciting antiviral activity in a host, said method comprising:

a) preparing a compound AV-L1 or AV-L2 wherein AV is an antiviral compound peptide with a mass of less than 60 kD and L1 or L2 is a linker covalently bound to AV;

b) treating the compound AV-L1 or AV-L2 with isolated one or more protein Pr ex vivo for a time sufficient for the compound AV-L1 or AV-L2 to covalently bond to one or more of the isolated proteins to form one or more modified protein complex of the above embodiments and variations; and

c) administering the modified protein or proteins to the host.

In one variation of the embodiment, the protein is albumin. In another variation, the albumin is obtained from blood, purified and isolated from blood prior to treating with the compound AV-L1 or AV-L2. In yet another variation, the albumin is HSA or recombinant HSA.

In one embodiment, the invention provides a pharmaceutical composition comprising a therapeutically effective amount of a complex of the above embodiments and variations, or a physiologically acceptable salt thereof, and a pharmaceutically acceptable carrier, excipient, or diluent.

In one embodiment, the invention provides a process for inhibiting the action of the HIV virus which process comprises administering to a host in recognized need of such treatment an effective amount of a complex of the above embodiments and variations, or a pharmaceutically acceptable salt thereof.

In one variation, the invention provides a method of treating a subject suffering from a viral infection, comprising administering to said subject an effective amount of a composition of the above embodiments and variations. According to the above variations, the subject is suffering from HIV infection.

In another embodiment, the invention provides a method of prophylaxis in a patient suspected of being exposed to a viral infection, comprising administering to said subject an effective amount of a composition of the above variations.

Methods of Treatment

The present invention takes advantage of the properties of existing anti-viral agents. The viruses that may be inhibited by the compounds of the present invention include, but are not limited to all strains of viruses listed, e.g., in U.S. Pat. No. 6,013,263 and U.S. Pat. No. 6,017,536 at Tables V-VII and IX-XIV therein. These viruses include, e.g., human retroviruses, including HIV-1, HIV-2, and human T-lymphocyte viruses (HTLV-I and HTLV-II), and non-human retroviruses, including bovine leukosis virus, feline sarcoma virus, feline leukemia virus, simian immunodeficiency virus (SIV), simian sarcoma virus, simian leukemia, and sheep progress pneumonia virus. Non-retroviral viruses may also be inhibited by the compounds of the invention, for example human respiratory syncytial virus (RSV), canine distemper virus, Newcastle Disease virus, human parainfluenza virus (HPIV), influenza viruses, measles viruses (MeV), Epstein-Barr viruses, hepatitis B viruses, and simian Mason-Pfizer viruses. Non-enveloped viruses may also be inhibited, and include, but are not limited to, picomaviruses such as polio viruses, hepatitis A virus, enteroviruses, echoviruses, coxsackie viruses, papovaviruses such as papilloma virus, parvoviruses, adenoviruses, and reoviruses.

The compounds of the present invention may be administered to patients according to the methods described below and other methods known in the art. Effective therapeutic dosages of the compounds derivatives may be determined through procedures well known by those in the art.

The compounds also can be administered prophylactically to previously uninfected individuals. This can be advantageous in cases where an individual has been exposed to a virus, as can occur when individual has been in contact with an infected individual where there is a high risk of viral transmission. This can be especially advantageous where there is no known cure for the virus, such as the HIV virus. As an example, prophylactic administration of a compound of the invention would be advantageous in a situation where a health care worker has been exposed to blood from an HIV-infected individual, or in other situations where an individual engaged in high-risk activities that potentially expose that individual to the HIV virus.

The preferred route of administration of the compounds of the invention is via intravenous administration, which allows the compounds to circulate in the bloodstream and reach their desired target. However, the methods of the present invention comprehend any method of administration that permits circulation of the compounds in the body of the patient.

The compounds and pharmaceutical compositions of the present invention may be used alone or in combination with other anti-viral compounds. For example, compounds and pharmaceutical compositions of the present invention may be used in a variety of drug ‘cocktails’, or combinations of three or more antiretroviral agents, that can potently suppress viral replication and prevent or delay the onset of AIDS.

The invention having been fully described can be further appreciated and understood with reference to the following non-limiting examples.

Compounds according to the present invention may optionally be synthesized according to the following general reaction schemes:
Preparation of Complex of Formula I: 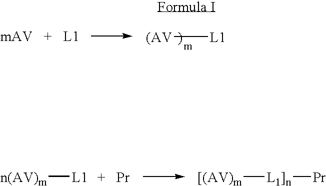
Preparation of Complex of Formula II:
L2+oPr→L2-(Pr)ₒ
pL2-(Pr)ₒ+AV→AV-[L2-(Pr)ₒ]ₚ Formula II

As shown in the Scheme above for the preparation of the complex of Formula I, the antiviral is first attached to the linker L1 to form the antiviral-linker AV-L1, which is followed by the reaction with protein Pr to form the complex of Formula I. However, it is also feasible to first attach the linker L1 to the protein Pr to form a linker-protein compound, L1 -Pr, which is then linked with the antiviral agent to form the complex of formula I. Similarly, the present invention also teaches that the reverse sequence as noted above may also be applicable for the preparation of the complex of Formula II.

### EXAMPLE 1

### Design and Preparation of HIV Fusion Inhibitor Peptides

Sequences of putative HIV fusion inhibitor peptides were modeled using the crystal structure of the gp41 trimeric helical fiisogenic complex (reviewed in Jiang et al, 2002). Peptide sequences were modeled to form helical segments that can fit into the grooves formed by the N-terminal triple helical core of the fusogenic complex. Evaluation of the inner binding and outer exposed surfaces of the modeled helical peptides were used to determine the sequence and composition of amino acids in the model peptides. Amino acids that have exposed side chains after complex formation are varied to improve solubility and other physical-chemical characteristics of the model peptide. Amino acids that bind to the N-terminal triple helical core are determinative of binding affinity and antiviral activity.

Most of the peptides in Tables 1 and 2 reflect changes in surface residues and were predicted to be equally potent antiviral compounds against the HIV HXB2 strain. All of the peptides in Table 1 have an acetyl group at the N-terminus and a C-terminal amide.

The peptides were prepared using standard solid phase techniques on Tentagel-S-RAM resin (Rapp Polymer), 0.25 mmol/g. All gave HPLC purities>90% and correct mass spec.

Synthesis Protocol on Resin:

1. Deprotection-25% piperidine/DMF (5+25 min)

2. Washing—DMF (6×1 min)

3. Coupling—3 eq. Fmoc-amino acid+3 eq. TCTU+6 eq. DEA to negative Kaiser test, (approx. 1 h); 3 h for Asn³¹ and Lys³³

4. Washing—DMF (5×1 min)

5. Terminal acetylation—Acetic anhydride/DEEA

Cleavage From Resin:

TFA—m-cresol—thioanisol—triisopropylsilane (85:5:5:5) 2 h, RT evaporation in vacuum

precipitation by ether (crude yield ˜80%)

Purification:

HPLC using a Biosphere C-18 column

mobile phase—A: water/0.1% TFA B: acetonitrile/0.1% TFA

gradient—10-20% B/10 min, 20-40% B/90 min

detection—UV 220 nm

Fractions over 95% were collected and lyophilized

Analysis:

HPLC: column—Luna C 18
mobile phase—A: water/0.04% H₃PO₄ B: acetonitrile/0.04% H₃PO₄
gradient—5-65% B/30 min
detection—UV 220 nm

MS[MH]⁺

TCTU=O-(1H-6-Chlorobenzotriazole-1-yl)-1,1,3,3-tetramethyluronium tetrafluoroborate

DIEA=diisopropylethylamine

### EXAMPLE 2

### Evaluation of Antiviral Activity of Peptides

Antiviral potency of the peptides was analyzed against HIV-1 HXB2 or NLA-3 strains using a cytotoxicity assay with MT4 cells as previously described (ref 1-3) with minor modifications. MT-4 cells (1.5×10⁴/ml) were exposed to 200 50% tissue culture infective doses (TCID50) of viruses in the presence of various concentrations of test compound in 96 well microtiter plates and incubated at 37° C. for 5 days. Cytotoxicity of HIV was measured by the addition of 3-(4,5-dimethylthiazol-2-yl)-2,5-diphenytetrazolium bromide (MMI) solution to each well to a final concentration of 0.75 mg/ml, and incubation for 1 hour a 37° C. After incubation, cells were dissolved in isopropanoyrriton-X 100/HCl (1000:50:25) solution. Absorbance was monitored in a microplate reader (Spectramax, Molecular Devices) at 540 rm and 690 nm. MT-4 cells were obtained from the AIDS Research and Reference Reagent Program (ARRRP, Division of AIDS, NIAID, NIH: MT-4 from Dr. D. Richman). Cells were propagated in RPMI 1640 growth medium supplemented with 10% fetal bovine serum, 50U of penicillin and 50 μg of streptomycin per ml ([Ivitrogen, Carlsbad Calif.). IC₅₀ values for all compounds tested are listed in Table 1.

References

1. Kodama, E., S. Shigeta, T. Sizuzki, E. De Clerq. 1996. Application of a gastric cancer cell line (MKN-28) for anti-adenovirus screening using the MTT method. Antiviral Res. 31:159-164.

2. Pauwels, R., J. Balzarini, M. Baba, R.Snoeck, D. Schols, P. Herdeweijn, J. Desmyter, E. De Clerq. 1988. Rapid and automated tetrazolium-based colorimetric assay for the detection of anti-HIV compounds. J. Virol. Methods. 20:309-321.

3. Yoshimura, K., R. Kato, M. F. Kavlick, A. Nguyen, V. Maroun, K. Maeda, K. A. Hussain, A. K. Ghosh, S. V. Gulnik, J. W. Erickson, H. Mitsuya. 2002. A potent HIV-1 protease inhibitor, UIC-94003(TMC-126), and selection of novel (A28S) mutation in the protease active site. J.Virol. 76:1349-1358.

TABLE 1
Antiviral activity of unmodified peptides

IC₅₀* (nM)

| | |
|---|---|
| HIV-1 | HIV |
| IIIB | NL4-3 |

| | | | |
|---|---|---|---|
| peptide 2 | Ac-W-X-E-W-D-R-E-Q-N-N-Y- | 2.4 | 18 |
| | T-S-L-I-H-S-L-I-E-E-S-Q-N- | | |
| | Q-Q-E-K-N-E-Q-E-L-L-NH2 | | |
| peptide 3 | Ac-W-X-E-W-D-R-E-W-N-N-Y- | 1.7 | 7.6 |
| | T-S-L-I-H-S-L-I-E-E-S-Q-N- | | |
| | Q-Q-E-K-N-E-Q-E-L-L-NH2 | | |
| peptide 4 | Ac-W-X-E-W-D-R-E-I-N-N-Y- | 2.0 | 9.5 |
| | T-S-L-I-H-S-Y-E-E-S-Q-N-Q- | | |
| | Q-E-K-N-E-Q-E-L-L-NH2 | | |
| peptide 5 | Ac-W-X-E-W-D-R-E-I-N-N-Y- | 6.6 | 200 |
| | T-S-L-I-H-S-Q-I-E-E-S-Q-N- | | |
| | Q-Q-E-K-N-E-Q-E-L-L-NH2 | | |
| peptide 6 | Ac-W-X-E-W-D-R-K-I-E-E-Y- | 2.4 | 1.2 |
| | T-K-K-I-K-K-L-I-E-E-S-Q-E- | | |
| | Q-Q-E-K-N-E-K-E-L-K-NH2 | | |
| peptide 7 | Ac-W-X-E-W-D-R-K-I-E-E-Y- | 2.4 | 1.1 |
| | T-K-K-I-K-K-L-I-E-E-S-Q-E- | | |
| | L-Q-E-K-N-E-K-E-L-K-NH2 | | |
| peptide 8 | Ac-W-X-E-W-D-R-K-I-E-E-Y- | 2.6 | 1.2 |
| | T-K-K-I-K-K-L-I-E-E-A-Q-E- | | |
| | Q-Q-E-K-N-E-K-E-L-K-NH2 | | |
| peptide 9 | Ac-W-X-E-W-D-R-K-I-E-E-Y- | 1.7 | 0.9 |
| | T-K-K-I-E-E-L-I-K-K-S-Q-E- | | |
| | Q-Q-E-K-N-E-K-E-L-K-NH2 | | |
| peptide 10 | Ac-W-X-E-W-D-R-K-I-E-E-Y- | 2.0 | 1.2 |
| | T-K-K-I-E-E-L-I-K-K-S-Q-E- | | |
| | L-Q-E-K-N-E-K-E-L-E-K-NH2 | | |
| peptide 11 | Ac-W-X-E-W-D-R-K-I-E-E-Y- | 2.1 | 1.1 |
| | T-K-K-I-E-E-L-I-K-K-A-Q-E- | | |
| | Q-Q-E-K-N-E-K-E-L-K-NH2 | | |
| T20 | | 4.7 | 120 |
| T1249 | | 2.0 | 1.5 |
| c34 | | 1.4 | 1.7 |
| SIV c34 | | 3.8 | 72 |

*average of 2 or 3 experiments
Ac = acetyl

### EXAMPLE 3a

### Preparation of Chemically-reactive Modified HIV Fusion Inhibitor Peptides

Analogues of peptides 2 and 7 demonstrate the general applicability of the procedure for enhancing the pharmacokinetic activity of peptides with diverse sequences. SPI-30014 and SPI-70038 (see below, Table 2) were prepared using solid phase synthesis techniques as described above. Instead of acetylating the N-terminus it is reacted with Fmoc-8-amino-3,6-dioxaoctanoic acid, TCTU, DIEA for 3 h, washed as above and then reacted with 3-maleimidopropionic acid, TCTU, DIEA for 3 h. Cleavage and purification was as described above.

SPI-30014 MH⁺4545

SPI-70038 MH⁺4673

### EXAMPLE 3b

### Preparation of Quenched, Modified HIV Fusion Inhibitor Peptides

To generate unreactive controls maleirnide containing peptides were quenched using an excess of β-mercaptoethanol. The resulting quenched peptides (SPI-30014Q and SPI-70038Q) are unable to form covalent conjugates with HSA. The quenched derivatives were prepared as follows:

1 mg of SPI-30014 was dissolved in 22.0 ul DMSO. 5 ul of this solution was added to 45 ul of 53 mM aqueous 9-mercaptoethanol (final concentration 47.7 mM) and incubated for 5 hours at 37° C. The molar ratio of peptide: β-mercaptoethanol was 1:51 and the final concentration of peptide was 0.9 mM.

Similarly 1 mg of SPI-70038 was dissolved in 21.4 ul DMSO. 5 ul of this solution was added to 45 ul of 53 mM aqueous β-mercaptoethanol (final concentration 47.7 mM) and incubated for 5 hours at 37° C. The molar ratio of peptide: β-mercaptoethanol was 1:58 and the final concentration of peptide was 0.8 mM.

### EXAMPLE 4

### Preparation of Long-acting HIV Fusion Inhibitor HSA-peptide Conjugates

1 mg of SPI-30014 was dissolved in 22.0 ul DMSO. 10 ul of this solution was added to 90 ul 25% HSA (Seracare) and incubated for 5 hours at 37° C. The molar ratio of peptide: HSA in the final reaction mixture is approximately 1:4 and final peptide concentration is 0.9 mM.

Similarly 1 mg of SPI-70038 was dissolved in 21.4 ul DMSO. 10 ul of this solution was added to 90 ul 25% HSA (Seracare) and incubated for 5 hours at 37° C. The molar ratio of peptide: HSA in the final reaction mixture is approximately 1:4 and final peptide concentration is 0.8 mM.

The resulting HSA-peptide conjugates, SPI-30014HSA and SPI-70038HSA, were tested for antiviral and pharmacokinetic profile compared to the reactive intermediates (SPI-30014 and SPI-70038) and the quenched, unconjugated peptides (SPI-30014Q and SPI-70038Q).

### EXAMPLE 5

### Evaluation of Antiviral Activity of Modified and Conjugated HIV Fusion Inhibitor Peptides

Antiviral potency of maleimide containing peptides; quenched, unconjugated peptides; and, peptide-HSA conjugates was analyzed against HBX2 HIV-1 using a cytotoxicity assay with MT4 cells as described in Example 2. IC₅₀ values for these compounds are listed in Table 2. The activities of the reactive and quenched peptides are similar. The antiviral activity of each HSA-peptide conjugates is reduced by about 34 fold.

TABLE 2
Antiviral activity of modified peptides and conjugates

| | | IC₅₀ (nM)* |
|---|---|---|
| SPI-30014 | 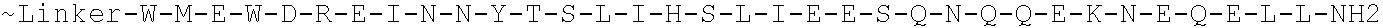 | 1.9 |
| SPI-30014Q | 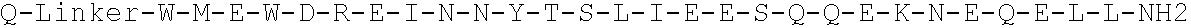 | 1.2 |
| SPI-30014HSA | 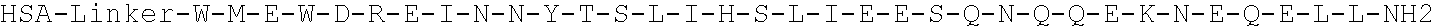 | 7.8 |
| SPI-70038 | 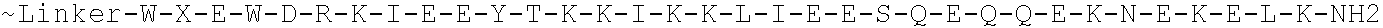 | 2.0 |
| SPI-70038Q | 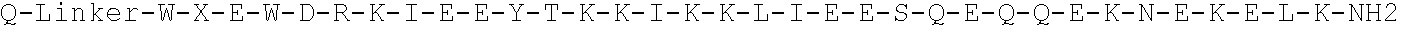 | 2.5 |
| SPI-70038HSA | 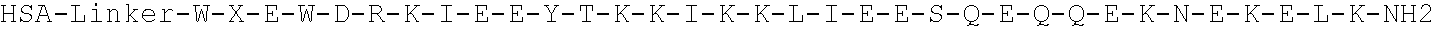 | 7.9 |

*average of 2-3 experiments
˜Linker - refers to maleimidopropionylaminoethoxyethoxyacetyl

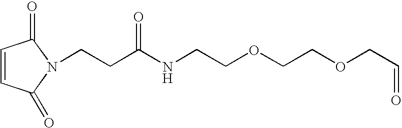

Q-Linker refers to maleimidopropionylaminoethoxyethoxyacetyl derivative quenched using β-mercaptoethanol

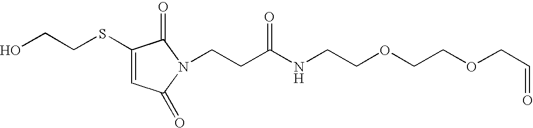

X — Norleucine (unnatural amino acid)

### EXAMPLE 6

### Evaluation of Pharmacokinetic Properties of Long-acting HIV Fusion Inhibitors

Peptide-HSA conjugates (SPI-30014HSA and SPI-70038HSA) and quenched, unconjugated peptides (SPI-30014Q and SPI-70038Q) were administered intravenously to Sprague-Dawley rats weighing between 400-500 g. Test compounds were formulated in DMSO (peptides) or phosphate-buffered saline (HSA-peptide conjugates) and were administered intravenously in a single dose of 0.5-0.6 umol/kg; rate of infusion was 2.0 ml/min; total infusion time was about 20 sec. Serum samples were collected at 5 min, 30 min, 1, 2, 8, 24, 48, and 72 hours post dose. The concentrations of antiviral peptides and conjugates in the rat plasma were analyzed using a cell-based antiviral bioassay. For each time point the serum sample was initially diluted 1:10 with growth medium and subsequently used for multiple serial dilutions analyzed in the standard antiviral assay using MT4 cells and HIV-1 HXB2 virus described above. The IC₅₀ value was determined and expressed as the percent of sample serum necessary to inhibit 50% of the cytotoxic activity of HIV-1 HXB2. A reference sample was prepared that contains predose serum of the corresponding animal diluted 10-fold by medium. To this diluted sample a defined concentration of peptide or peptide-HSA conjugate in aqueous DMSO was added. This sample was then analyzed in the antiviral assay in the same way as test time point samples. This IC₅₀ reference value was determined and expressed as a concentration of peptide or peptide-HSA conjugate. The concentration of peptide or peptide-HSA conjugate in each test time point serum sample was then calculated based on the IC₅₀ values obtained with the reference sample in the serum from the corresponding animal:
Concentration (nM)=[IC₅₀ ref(nM)/IC₅₀(% of sample serum)]×100%

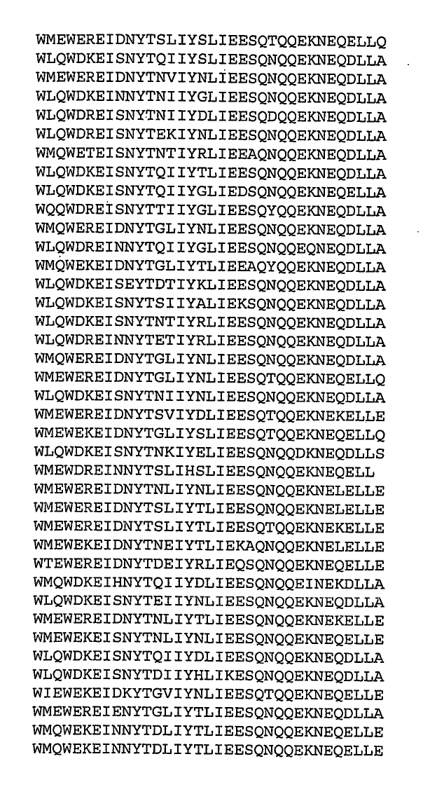

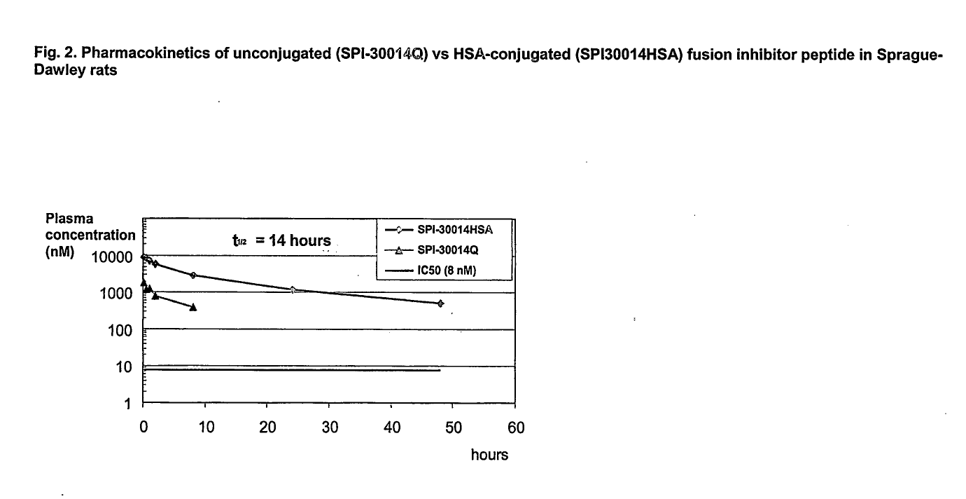

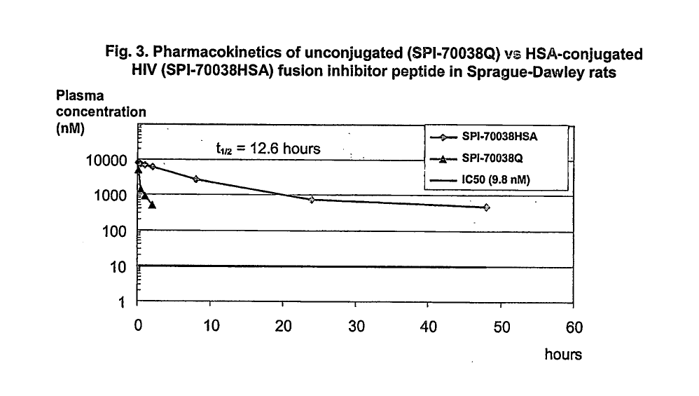

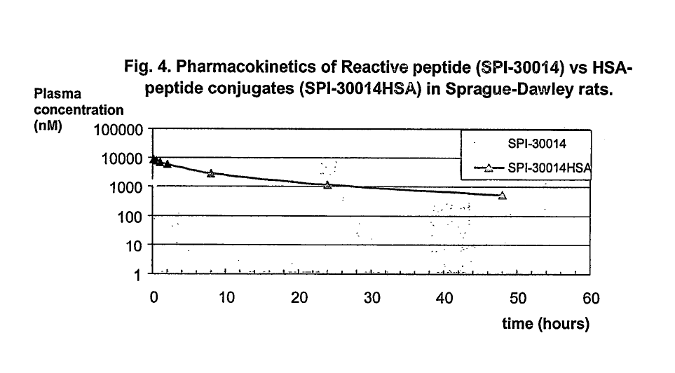

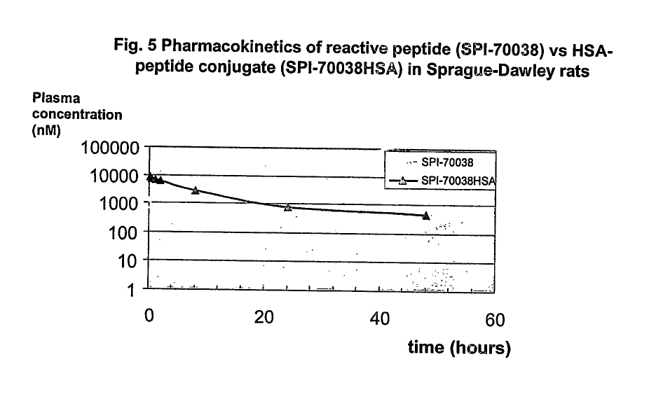

The concentration vs time profiles of test compounds in rat plasma are shown in FIGS. 2 and 3 (average of 2-3 rats). The unconjugated (control) peptides display a rapid clearance profile: by 8 hours 80% of peptide is lost. In contrast, the terminal half-life of the two HSA-peptide conjugates ranged from 12 to 14 hours. The half-life of the HSA-peptide conjugates is similar to the reported half-life of HSA alone in rodents (15.8 hours) (1 ). I. M. Gaizutis, Pesce A. J., Pollak V. E. 1975. Renal clearance of human and rat albumins in the rat. Proc. Soc. Exp. Biol. Med. 148(4):947-952.

These results indicate that the half-life, distribution, and elimination of the antiviral compounds was determined by the cloaking protein (HSA), while the antiviral activity was determined by the warhead peptide. The prolongation of plasma activity in the animal is unexpected in light of the retention of potent biological activity of the conjugate. The latter finding would suggest that the warhead portion of the molecule is clearly exposed and therefore would be expected to be subject to metabolism, degradation, elimination and clearance processes in the body, but our results suggest that it is protected from these processes.

### EXAMPLE 7

Conjugation Reaction to Form the Complex of the Invention:

A 10 mM solution of maleimidopropionylaminoethoxyethoxyacetyl derivatized antiviral peptide in DMSO was added to a 25% water solution of HSA. The final peptide concentration in the reaction mixture was 1 mM and the molar ratio in reaction mixture of peptide: HSA was 1:4. The solution was incubated 5 hours at 37° C. Once incubation was complete, the conjugate was stored at 4° C.

### EXAMPLE 8

Conjugation Reaction to Form the Complex of the Invention:

A 10 mM solution of trans-4-(maleimidylmethyl)cyclohexane-1-carbonyl derivatized antiviral peptide in DMSO is added to a 25% water solution of HSA. The final peptide concentration in the reaction mixture is 1 mM and the molar ratio in reaction mixture of peptide: HSA is 1:4. The solution is incubated 5 hours at 37° C. Once incubation is complete, the conjugate is stored at 4° C.

### EXAMPLE 9

Conjugation Reaction to Form the Complex of the Invention:

A 10 mM solution of N-(3-{2-[2-(3-amino-propoxy)-ethoxy]-ethoxy]-ethoxy}-propyl)-2-bromoacetamide derivatized antiviral peptide in DMSO is added to a 25% water solution of HSA. The final peptide concentration in the reaction mixture is 1 mM and the molar ratio in reaction mixture of peptide: HSA is 1:4. The solution is incubated 5 hours at 37° C. Once incubation was complete, the conjugate is stored at 4° C.

### EXAMPLE 10

Conjugation Reaction to Form the Complex of the Invention:

A 10 mM solution of maleimidopropionylaminoethoxyethoxyacetyl derivatized antiviral peptide in DMSO is added to a 25% water solution of HSA. The final peptide concentration in the reaction mixture is 1 mM and the molar ratio in reaction mixture of peptide: HSA is 1:1. The solution is incubated 5 hours at 37° C. Once incubation is complete, the conjugate is stored at 4° C.

### EXAMPLE 11

Conjugation Reaction to Form the Complex of the Invention:

A 10 mM solution of maleimidopropionylaminoethoxyethoxyacetyl derivatized antiviral peptide in DMSO is added to a 25% water solution of HSA. The final peptide concentration in the reaction mixture is 1 mM and the molar ratio in reaction mixture of peptide: HSA is 10:1. The solution is incubated 5 hours at 37° C. Once incubation is complete, the conjugate is stored at 4° C.

### ASSAY EXAMPLES

### EXAMPLE 12

### Binding Assay for Peptidyl Inhibitors of IV Fusion

The binding affinity of conjugated inhibitors of HIV fuision is measured using a chimeric peptide (IQN17) that contains a segment of GCN4 at the N-terminal and 17 residues from the first heptad repeat region of HIV-1 GP41 at the C-terminal (Cell 99, 103-115). A 28-residue peptide from the second heptad repeat region of GP41 (C28) is labeled with a fluorescent molecule Alexa-430 (Molecular Probes) at its carboxyl terminal. The binding is measured by titration of labeled C28 with IQN17. The concentrations of bound and unbound C28 were measured by capillary zone electrophoresis. At 3 μM C28 and 8 μm IQN17, about 80% C28 is bound to IQN17. For unlabeled peptides, the amount that competes 50 % C28 off IQN17 gives its IC50 value.

Renin Inhibitor Compounds and Complexes

The present invention relates to biologically active compounds that may be used to react with proteins to form covalently linked complexes wherein the resulting complexes are found to exhibit renin inhibition activities in vivo. More specifically, the complexes are isolated complexes comprising a renin inhibitor and a linkig group, and the blood component is a protein such as albumin. The present invention also provides methods for inhibiting renin activity in vivo comprising administering to the bloodstream of a mammalian host the novel isolated complexes of the present invention.

In one embodiment, a pharmaceutical composition is provided that comprises a purified renin inhibitor complex according to the present invention as an active ingredient. Pharmaceutical compositions according to the invention may optionally comprise 0.001%-100% of one or more renin inhibitors complexes of this invention. These pharmaceutical compositions may be admninistered or coadministered by various methods known in the art for administering biologically active agents to the bloodstream. In a preferred aspect of the invention, the compositions may be administered by injection. In another preferred aspect, the compositions may be administered by infusion.

In another embodiment, methods and compositions are provided for delivery of isolated conjugated complexes comprising biologically active agents, particularly therapeutic agents such as renin inhibitors, where the complexes comprising the agents have an extended half-life in the bloodstream as compared to non-conjugated agents.

The invention comprises using a biologically active compound covalently attached or linked to a linking group, the linking group comprising at least one chemically reactive moiety which is capable of forming covalent bonds with functionalities present on the protein. By preparing the isolated complexes before administration of the complexes into the blood of the host, particularly the bloodstream of the host, a biologically active complex is generated that maintain an effective therapeutic effect in the bloodstream for an extended period of time as compared to a non-conjugated biologically active agent.

In one embodiment, the invention provides an isolated complex of the Formula I or Formula II:
[(Ih)ₘ-L1]ₙ-Pr I
Ih-[L2-(Pr)ₒ]ₚ II

wherein:

m is an integer from 1-5;

n is an integer from 1-100;

o is an integer from 1-5;

p is an integer from 1-100;

Ih is a renin inhibitor;

L1 and L2 are polyvalent linkers covalently linking Th to Pr, or where L1 and L2areabsent;

Pr is a protein; and

wherein the complex possesses renin inhibitory activity in vivo.

In another embodiment, the renin inhibitor Ih is a peptide. In another embodiment, the peptide has a mass of less than about 60 kDA. In another embodiment, the peptide has a mass of less than about 10 kDA. In yet another embodiment, the peptide has a mass of less than about 1000 DA.

In one particular embodiment, the peptide is a peptide mimetic. In another embodiment, the peptide is a transition state mimetic at the C-terminus.

In one embodiment, the transition state mimetic at the C-terminus is selected from the group consisting of

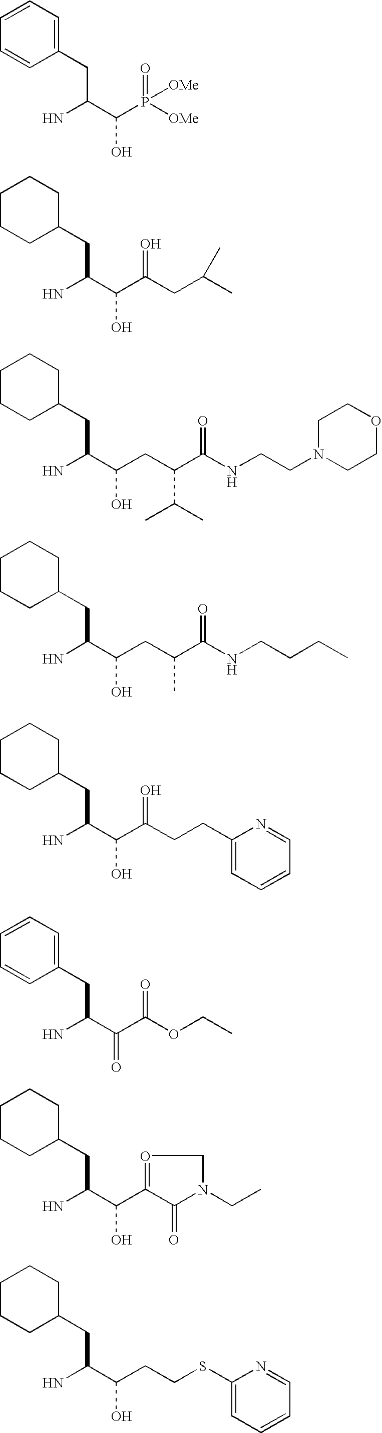

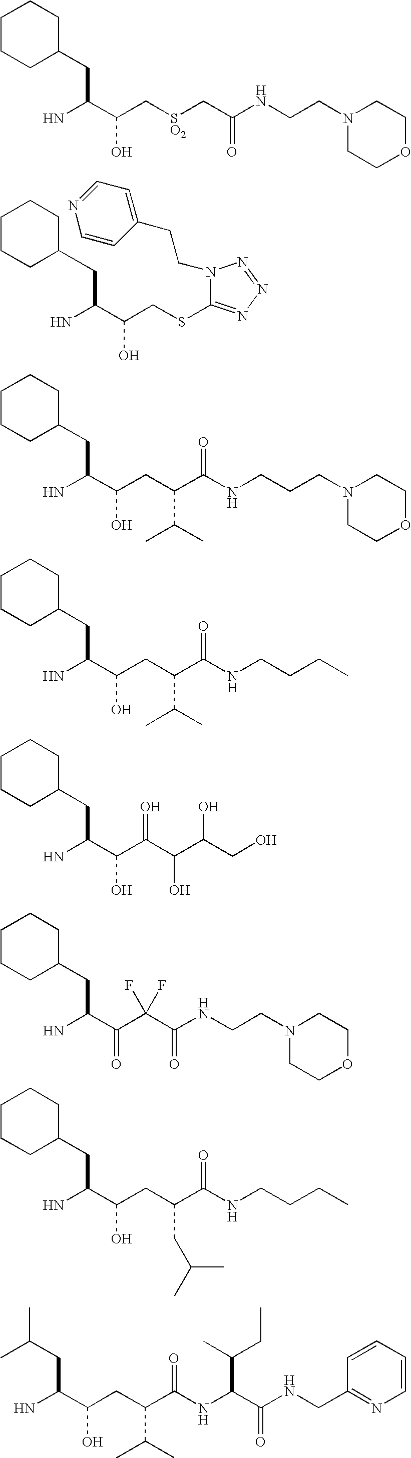

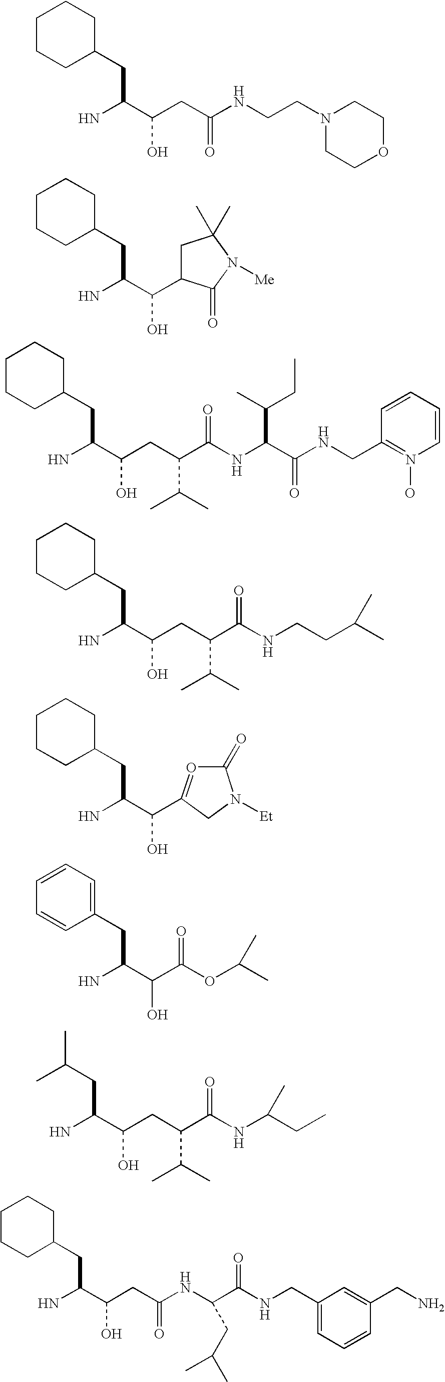

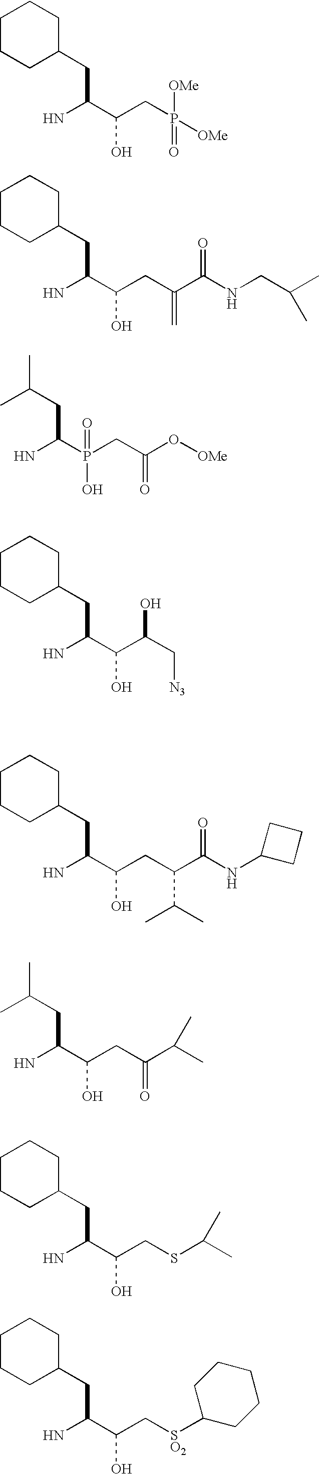

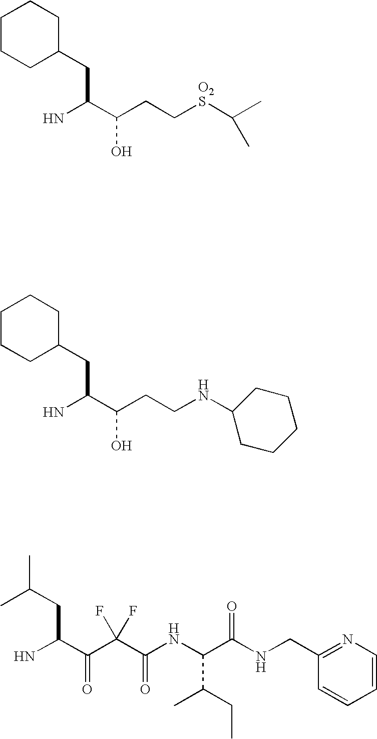

In another embodiment, Ih is a renin inhibitor peptide selected from the group consisting of Iva-Val-Val-Sta-Ala-Sta, Boc-Phe-His-Sta-Ile-AMP, Boc-Phe-His-Sta-Ala-Sta-OMe, Boc-Phe-His-Sta-Leu—NHCH2Ph, Boc-Phe-His-ACHPA-Leu-AMB, Boc-Phe-His-Sta-Leu-AMB, Boc-Pro-Phe-His-Sta-Ile-AMP, Iva-Phe-NMe-Sta-Ala-Sta, Iva-His-Pro-Phe-His-Sta-Ala-Sta, Iva-His-Pro-Phe-His-Sta-Leu-Phe-NH2, Ac-His-Pro-Phe-Val-Sta-Leu-Phe-NH2, Ac-His-Pro-Phe-His-ACHPA-Leu-Phe-NH2, Ac-Trp-Pro-Phe-His-Sta-Ile—NH2, Ac-(HCO-Trp)-Pro-Phe-His-Sta-Ile-NH2, Pro-His-Pro-Phe-His-Sta-Ile-His-D-Lys, Pro-His-Pro-Phe-His-Sta-Ile-Phe-NH2, Z-Arg-Arg-Pro-Phe-His-Sta-Ile-His-Lys(Boc)-OMe, Pro-His-Pro-Phe-His-Phe-Phe-Val-Tyr-Lys, His-Pro-Phe-His-Leu-D-Leu-Val-Tyr—OH, Pro-His-Pro-Phe-His-Leu(CH2NH)Val-Ile-His-Lys (H-142), Boc-Phe-His-Cha-(CH2NH)Val-NH2(S)-Me(Bu), Pro-His-Pro-Phe-His-Leu-Phe-Val-Tyr-OH, Boc-His-Pro-Phe-His-Leu(CH(OH)CH2)Val-Ile-His—OH (H-26 1), and PEC-Phe-His-ACHPA-ILeNHC(CH2OH)2CH3.

In another embodiment, Ih is a renin inhibitor peptide selected from the group consisting of Iva-Val-Val-Sta-Ala-Sta, Boc-Phe-His-Sta-Ile-AMP, Boc-Phe-His-Sta-Ala-Sta-OMe, Boc-Phe-His-Sta-Leu-NHCH2Ph, Boc-Phe-His-ACHPA-Leu-AMB, Boc-Phe-His-Sta-Leu-AMB, Boc-Pro-Phe-His-Sta-Ile-AMP, Iva-Phe-Noe-Sta-Ala-Sta, Iva-His-Pro-Phe-His-Sta-Ala-Sta, Iva-His-Pro-Phe-His-Sta-Leu-Phe-NH2, Ac-His-Pro-Phe-Val-Sta-Leu-Phe-NH2, Ac-His-Pro-Phe-His-ACHPA-Leu-Phe-NH2, Ac-Trp-Pro-Phe-His-Sta-Ile-NH2, Ac-(HCO-Trp)-Pro-Phe-His-Sta-Ile-NH2, Pro-His-Pro-Phe-His-Sta-Ile-His-D-Lys, Pro-His-Pro-Phe-His-Sta-Ile-Phe-NH2, Z-Arg-Arg-Pro-Phe-His-Sta-Ile-His-Lys(Boc)-OMe, Pro-His-Pro-Phe-His-Phe-Phe-Val-Tyr-Lys, His-Pro-Phe-His-Leu-D-Leu-Val-Tyr-OH, Pro-His-Pro-Phe-His-Leu(CH2NH)Val-Ile-His-Lys (H-142), Boc-Phe-His-Cha-(CH2NH)Val-NH-2(S)-Me (Bu), Pro-His-Pro-Phe-His-Leu-Phe-Val-Tyr-OH, Boc-His-Pro-Phe-His-Leu(CH(OH)CH2)Val-Ile-His—OH (H-26 1), and PEC-Phe-His-ACHPA-ILeNHC(CH2OH)2CH3, and Pr is albumin.

In a particular embodiment, the linker L1 or L2 comprises at least two functional groups covalently linking Ih to Pr. In another embodiment, the linker L1 or L2 is hydrolytically stable in human serum for an extended period of time. In particular, the linker is sufficiently hydrolytically stable that, when administered to a subject, the active conjugate produces a sustained decrease in blood pressure over an extended period of time. In particular embodiments, the linker is sufficiently stable that the conjugate can produce a sustained decrease in blood pressure for 1 day, 2 days, 3 days, 4 days, 5 days, 6 days, 7 days or more, or 14 days or more. In yet another embodiment, the linker L1 or L2 is stable in human serum for half lives of between 8 hours and 30 days.

In another embodiment of the invention, the linker L1 or L2 is a derivative of a compound selected from the group consisting of acyloxymethylketones, aziridines, diazomethyl ketones, epoxides, iodo-, bromo- or chloroacetamides, α-haloesters, α-haloketones, sulfoniums, chloroethylsulfides, O-alkylisoureas, alkyl halides, vinylsulfones, acrylamides, acrylates, vinylpyridines, organometallic compounds, aryldisulfides, thiosulfonates, aldehydes, nitriles, α-diketones, α-ketoamides, α-ketoesters, diaminoketones, semicarbazones, and dihydrazides.

In one embodiment, the linker L1 or L2 is a derivative of a compound selected from the group consisting of azidobenzoyl hydrazide, N-[4-(p-azidosalicylamino)butyl]-3′-[2′-pyridyldithio)propionamide), bis-sulfosuccinimidyl suberate, dimethyl adipimidate, disuccinimidyl tartrate, N-y-maleimidobutyryloxysuccinimide ester, N-hydroxy sulfosuccinimidyl-4-azidobenzoate, N-succinimidyl [4-azidophenyl]-1,3′-dithiopropionate, N-succinimidyl[4-iodoacetyl]aminobenzoate, glutaraldehyde, succinimidyl 4-[N-maleimidomethyl]cyclohexane-1-carboxylate, N-hydroxysulfosuccinimide, maleimide-benzoyl-succinimide, emaleimido-butyryloxy succinimide ester, maleimidopropionic acid, N-hydroxysuccinimide, isocyanate, thioester, thionocarboxylic acid ester, imino ester, carbodiimide, anhydride and carbonate ester.

In one particular embodiment of the invention, the protein is selected from the group consisting of red blood cells, and immunoglobulins, such as IgM and IgG, serum albumin, transferrin, p90 and p38. In another particular variation, the protein is albumin. In another variation, the albumin is HSA or recombinant HSA that is at least 10% pure on a dry matter basis. In a further variation, the linkage is to a Cys-34 of human albumin. In yet another variation, the linkage is to a lysine of human albumin.

In one embodiment, the invention provides a complex of Formula I or Formula II, wherein m is 1, n is 1 or 2, and the protein is HSA or recombinant HSA. In another variation of the above embodiment, n is 1, the protein is HSA or recombinant HSA, and wherein the complex is further purified to a purity of at least 30%. In yet another variation, m is 1, n is 2, and the protein is HSA or recombinant HSA.

In one variation, the complex is prepared by combining a stoichiometric ratio of (Ih)m-L1 with Pr or a stoichiometric ratio of Ih with L2-(Pr)o. In another variation, the complex is prepared by combining a mixture of Pr to (Ih)m-L1 in a ratio of at least about 1.3:1.

In another embodiment, the invention provide the complex of Formula I or Formula II wherein L1 and L2 are absent, and wherein the complex is prepared by forming an activated intermediate of Ih followed by the condensation of the activated Ih intermediate with Pr. In another variation, the activated intermediate of Ih is prepared from a mixed anhydride or N,N′-carbonyldiimidazole reagent. Optionally, in the above variations, the complex may be further purified to a purity of at least about 30%.

In one embodiment of the invention, the renin inhibitor is a peptidomimetic with a mass of less than about 1000 DA.

In another embodiment, there is provided a composition comprising the complex of Formula I or Formula II and a physiologically acceptable carrier. In another embodiment, the composition above is formulated for parenteral administration. In another embodiment, the composition above is selected from the group consisting of solutions, dry products for combining with a solvent prior to use, suspensions, emulsions, and liquid concentrates.

In another embodiment, the invention provides a method for inhibiting renin activity in vivo, said method comprising:

administering to the bloodstream of a mammalian host an isolated conjugate complex of Formula I or Formula II, wherein the complex is formed by attaching a renin inhibitor to a linker having at least one reactive functional group which reacts with the protein to form stable covalent bonds; and

wherein the isolated conjugate complex is administered in an amount to maintain an effective therapeutic effect in the bloodstream for an extended period of time as compared to a non-conjugated renin inhibitor.

In one variation, the invention provides a method wherein the complex is the complex according to any of the above complexes. In another variation of the above methods, the protein is HSA or recombinant HSA.

In one variation of the above methods, the linker comprises a reactive functional group is a compound selected from the group consisting of acyloxymethylketones, aziridines, diazomethyl ketones, epoxides, iodo-, bromo- or chloroacetamides, α-haloesters, oLhaloketones, sulfoniums, chloroethylsulfides, O-alkylisoureas, alkyl halides, vinylsulfones, acrylamides, acrylates, vinylpyridines, organometallic compounds, aryldisulfides, thiosulfonates, aldehydes, nitriles, a diketones, α-ketoamides, α-ketoesters, diaminoketones, semicarbazones, and dihydrazides.

In one variation, the invention provides a method for inhibiting renin activity in vivo, said method comprises:

administering into the bloodstream of a mammalian host the complex of Formula I or Formula II in an amount sufficient to provide an effective amount for renin inhibition;

whereby said complex is maintained in the bloodstream over an extended period of time as compared to the lifetime of unbound renin inhibitor.

In yet another embodiment, there is provided a method for inhibiting renin activity in a host, said method comprising:

a) preparing a compound Ih-L1 or Ih-L2 wherein Ih is a renin inhibitor peptide with a mass of less than 60 kD and L1 or L2 is a linker covalently bound to Ih;

b) treating the compound rh-L1 or Ih-L2 with isolated protein ex vivo for a time sufficient for the compound Ih-L1 or Ih-L2 to covalently bond to the protein to form the protein complex of Formula I or Formula II, and

c) administering the treated protein complex to the host

In one variation of the above method, the protein is albumin. In another variation, the albumin is HSA or recombinant HSA. In yet another variation of the above method, the albumin is obtained from blood, purified and isolated from blood prior to treating the albumin with the compound Ih-L1 or Ih-L2. In another variation, the albumin is purified to a purity level of at least 10% on a dry matter basis. In yet another variation, the albumin is purified to a purity level of more than 95%.

In another embodiment, the invention provides a method for inhibiting renin activity in a host, said method comprising:

a) preparing a compound Ih-L1 or Ih-L2 wherein Ih is a renin inhibitor peptide with a mass of less than 60 kD and L1 or L2 is a linker covalently bound to Ih;

b) treating the compound Ih-L1 or Ih-L2 with isolated one or more protein Pr ex vivo for a time sufficient for the compound Ih-L1 or Ih-L2 to covalently bond to one or more of the isolated proteins to form one or more modified protein complex of Formula I or Formula II; and

c) administering the modified protein or proteins to the host.

In one variation of the above method, the protein is albumin. In another variation, the albumin is obtained from blood, purified and isolated from blood prior to treating with the compound rh-L1 or Ih-L2. In yet another variation of the method, the albumin is HSA or recombinant HSA.

In one embodiment, there is provided a pharmaceutical composition comprising a therapeutically effective amount of a complex as describe above, or a physiologically acceptable salt thereof, and a pharmaceutically acceptable carrier, excipient, or diluent. In another variation, there is provided a method of reducing the blood pressure of a subject comprising administering to the subject a therapeutically effective amount of the above composition. In yet another variation, the invention provides the above method, wherein the patient suffers from hypertension. In yet another variation of the above method, the patient suffers from mild, moderate or severe hypertension.

In another embodiment of the invention, the transition state mimetic is a compound of the formula:

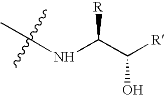

wherein:

R is selected from the group consisting of (C₁₋₁₀)alkyl, (C₆₋₁₂)cycloalkyl, carbonyl(C₁₋₁₀)alyl, sulfonyl(C₁₋₃)allyl, sulfnyl(C₁₋₃)alkyl, (C₂₋₁₂)alkenyl, (C₂₋₁₂)alkynyl, aryl, aryl(C₁₋₁₀)alkyl, heteroaryl, heteroaryl(C₁₋₁₀)alkyl, each substituted or unsubstituted; and

R′ is selected from the group consisting of (C₁₋₁₀)alkyl, (C₆₋₁₂)cycloalkyl, carbonyl(C₁₋₁₀)alkyl, (C₁₋₁₀)alkoxycarbonyl, (C₁₀)alkylaminocarbonyl, sulfonyl(C₁₋₃)alkyl, sulfinyl(C₁₋₃)alkyl, (C₂₋₁₂)alkenyl, (C₂₋₁₂)alkynyl, aryl, aryl(C₁₋₁₀)alkyl, heteroaryl, heteroaryl(C₁₋₁₀)alkyl, alkylsulfonyl(C₁₋₁₀)alkyl, arylsulfonyl(C₁₋₁₀)alkyl, heteroarylsulfonyl(C₁₋₁₀)alkyl, (C₁₋₁₀)alkylphosphonate and (C₁₋₁₀)alkyl phosphonyl, each substituted or unsubstituted.

In another embodiment, the transition state mimetic is a compound of the formula:

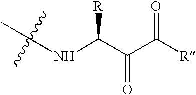

wherein:

R is selected from the group consisting of (C₁₋₁₀)alkyl, (C₆₋₁₂)cycloalkyl, carbonyl(C₁₋₁₀)alkyl, sulfonyl(C₁₋₃)alkyl, sulfinyl(C₁₋₃)alkyl, (C₂₋₁₂)alkenyl, (C₂₋₁₂)alkynyl, aryl, aryl(C₁₋₁₀)alkyl, heteroaryl, heteroaryl(C₁₋₁₀)alkyl, each substituted or unsubstituted; and

R″ is selected from the group consisting of (C₁₋₄)alkyl, (C₆₋₁₂)cycloalkyl, heterocycloalkyl, bicycloalkyl, carbonyl (C₁₋₁₀)alkyl, thiocarbonyl (C₁₋₃)alkyl, sulfonyl (C₁₋₃)alkyl, sulfinyl(C₁₋₃)alkyl, amino, imino(C₁₋₃)alkyl, (C₁₋₁₀)alkoxy, aryloxy, heteroaryloxy, (C₂₋₁₂)alkenyl, (C₂₋₁₂)alkynyl, aryl, aryl(C₁₋₁₀)alkyl, heteroaryl, heteroaryl(C₁₋₁₀)allyl, (C₉₋₁₂)bicycloaryl, hetero(C₈₋₁₂)bicycloaryl, aminosulfonyl, alkylsulfonyl, alkylsulfonyl(C₁₋₁₀)alkyl, arylsulfonyl, arylsulfonyl(C₁₋₁₀)alkyl, heteroarylsulfonyl, heteroarylsulfonyl(C₁₋₁₀)alkyl, phosphonate, (C₁₋₁₀)alkylphosphonyl, sulfonyl group and sulfinyl group, each substituted or unsubstituted.

In yet another embodiment, the transition state mimetic is a compound of the formula:

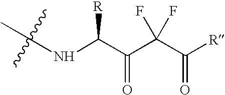

wherein:

R is selected from the group consisting of (C₁₋₁₀)alkyl, (C₆₋₁₂)cycloalkyl, carbonyl(C₁₋₁₀)alkyl, sulfonyl(C₁₋₃)alkyl, sulfinyl(C₁₋₃)alkyl, (C₂₋₁₂)alkenyl, (C₂₋₁₂)alkynyl, aryl, aryl(C₁₋₁₀)alkyl, heteroaryl, heteroaryl(C₁₋₁₀)alkyl, each substituted or unsubstituted; and

R″ is selected from the group consisting of (C₁₋₄)alkyl, (C₆₋₁₂)cycloalkyl, heterocycloalkyl, bicycloalkyl, carbonyl (C₁₋₁₀)alkyl, thiocarbonyl (C₁₋₃)alkyl, sulfonyl (C₁₋₃)alkyl, sulfinyl(C₁₋₃)alkyl, amino, imino(C₁₋₃)alkyl, (C₁₋₁₀)alkoxy, aryloxy, heteroaryloxy, (C₂₋₁₂)alkenyl, (C₂₋₁₂)alkynyl, aryl, aryl(C₁₋₁₀)alkyl, heteroaryl, heteroaryl(C₁₋₁₀)alkyl, (C₉₋₁₂)bicycloaryl, hetero(C₈₋₁₂)bicycloaryl, aminosulfonyl, alkylsulfonyl, alkylsulfonyl(C₁₋₁₀)alkcyl, arylsulfonyl, arylsulfonyl(C₁₋₁₀)alkyl, heteroarylsulfonyl, heteroarylsulfonyl(C₁₋₁₀)alkyl, phosphonate, (C₁₋₁₀)alkylphosphonyl, sulfonyl group and sulfinyl group, each substituted or unsubstituted.

In another embodiment, the transition state mimetic is a compound of the formula:

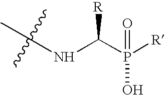

R is selected from the group consisting of (C₁₋₁₀)alkyl, (C₆₋₁₂)cycloalkyl, carbonyl(C₁₋₁₀)alkyl, sulfonyl(C₁₋₃)alkyl, sulfinyl(C₁₋₃)alkyl, (C₂₋₁₂)alkenyl, (C₂₋₁₂)alkynyl, aryl, aryl(C₁₋₁₀)alkyl, heteroaryl, heteroaryl(C₁₋₁₀)alkyl, each substituted or unsubstituted; and

R″ is selected from the group consisting of (C₁₋₄)alkyl, (C₆₋₁₂)cycloalkyl, heterocycloalkyl, bicycloalkyl, carbonyl (C₁₋₁₀)alkyl, thiocarbonyl (C₁₋₃)alkyl, sulfonyl (C₁₋₃)alkyl, sulfinyl(C₁₋₃)alkyl, amino, imino(C₁₋₃)alkyl, (C₁₋₁₀)alkoxy, aryloxy, heteroaryloxy, (C₂₋₁₂)alkenyl, (C₂₋₁₂)alkynyl, aryl, aryl(C₁₋₁₀)alkyl, heteroaryl, heteroaryl(C₁₋₁₀)alkyl, (C₉₋₁₂)bicycloaryl, and hetero(C₈₋₁₂)bicycloaryl, each substituted or unsubstituted.

The present invention relates to compounds and compositions that may be used as renin inhibitors with extended lifetime as compared to a non-conjugated renin inhibitor.

The invention comprises using a biologically active compourid covalently attached or linked to a linking group, the linking group comprising at least one chemically reactive moiety which is capable of forming covalent bonds with functionalities present on a protein or a blood protein. In one embodiment, the protein is albumin. By preparing the isolated complex ex vivo before the administration of the complex into the blood of the host, particularly the bloodstream of the host, the biologically active complex maintains an effective therapeutic effect in the bloodstream for an extended period of time as compared to a non-conjugated biologically active agent.

The extended life-time at a useful dosage will usually be at least 2 days, more preferably at least 5 days, even more preferably, at least 10 days, and most preferably at least 15 days. The protein that may be conjugated to include red blood cells, immunoglobulins, such as IgM and IgG, serum albumin, transferrin, p90 and p38. In a preferred embodiment, the protein is albumin. In another embodiment, the protein is recombinant albumin.

A large number of biologically active agents or therapeutic agents may be used as the conjugate with the protein. In a preferred embodiment, the biologically active or therapeutic agent Ih, is a renin inhibitor. The renin inhibitor, which may be depicted as Ih, comprises an active functional group that may be reacted with a linidng group, depicted as L1 or L2, to form an inhibitor-linking group compound, Ih-L1 or Ih-L2, which may react with one or more protein Pr. In one embodiment, the protein has a number of different functional groups which may react with the inhibitor-inking group compound to form a complex of Formula I:
[(Ih)ₘ-L1]ₙ-Pr I

In another embodiment, the protein has a number of different functional groups which may react with the inhibitor-linking group compound to form a complex of Formula II:
Ih-[L2-(Pr)ₒ]ₚ II

wherein Ih is a biologically active agent, L1 and L2 are linking groups that link Ih to Pr, Pr is a blood component, m and o are integers from 1-5, and n and p are integers from 1-100.

A number of functional groups are available on the protein such as albumin. Non-limiting functional groups include amino groups, carboxyl groups and thio groups. While any of these functional groups in the protein may be employed to form a covalent bond with the linker group, depending on the nature of the functional group(s) on the linking group and the linker, certain functional groups will be preferred over the others. For example, the reaction of amine groups may form conjugates having an amide group, carboxyl groups may form conjugates having an amide or ester groups, and thio groups may form thioethers or tioesters.

The Biologically Active Agents Ih:

The biologically active agent Ih may be any compound, such as an enzyme inhibitor, that will elicit a desired biological response and induce minimal immune response when administered in a mammalian host. Preferably, the biologically active agent is a renin inhibitor. More preferably, the agent is a peptide or peptidomimetic renin inhibitor. A large variety of renin inhibitors may be used in the present invention. Non-exclusive examples of peptide or peptidomimetic renin inhibitors are shown in the Table. Preferably, the renin inhibitors are peptidomimetics with a mass of less than about 60 kDA, more preferably less than about 10 kDA, and most preferably less than about 1000 DA.

TABLE
Peptide or Peptidomimetic Renin Inhibitors

| | | |
|---|---|---|
| | IC50 (nM) | |
| | Human | |
| Inhibitor | plasma renin | References |

| | | |
|---|---|---|
| Iva-Val-Val-Sta-Ala-Sta | 14000 | JMC 1152 (86) |
| (pepstatin) | | |
| Boc-Phe-His-Sta---Ile-AMP | 6 | JMC 1837 (87) |
| Boc-Phe-His-Sta---Ala-Sta-OMe | 27 | JMC 1152 (86) |
| Boc-Phe-His-Sta---Leu-NHCH₂Ph | 26 | JMC 1853 (87) |
| Boc-Phe-His-ACHPA---Leu-AMB | 1 | JMC 1918 (88) |
| Boc-Phe-His-Sta---Leu-AMB | 9 | JMC 1918 (88) |
| Boc-Pro-Phe-His-Sta---Ile-AMP | 4.1 | JMC 671 (88) |
| Iva-Phe-Nle-Sta-Ala-Sta | 28 | JMC 1152 (86), JMC 2287 (87) |
| Iva-His-Pro-Phe-His-Sta---Ala-Sta | 1.9 (Ki) | |
| Iva-His-Pro-Phe-His-Sta---Leu-Phe-NH₂ | 3 | JMC 1853 (87), JMC |
| | | 2080 (90), Nature 81 (83) |
| Ac-His-Pro-Phe-Val-Sta---Leu-Phe-NH₂ | 32 | JMC 1679 (88) |
| Ac-His-Pro-Phe-His-ACHPA---Leu-Phe-NH₂ | 0.5 | JMC 1679 (88) |
| Ac-Trp-Pro-Phe-His-Sta---Ile-NH₂ | 16 | JMC 18 (88) |
| Ac-(HCO-Trp)-Pro-Phe-His-Sta---Ile-NH₂ | 0.1 | JMC 18 (88) |
| Pro-His-Pro-Phe-His-Sta---Ile-His-D-Lys | 26 | JMC 1377 (88) |
| Pro-His-Pro-Phe-His-Sta---Ile-Phe-NH₂ | 3 | JMC 1287 (87) |
| Z-Arg-Arg-Pro-Phe-His-Sta--- | 1 | JMC 18 (88), |
| Ile-His-Lys(Boc)-OMe | | Hypertension 797 (85) |
| Pro-His-Pro-Phe-His-Phe-Phe- | 5200 | JMC 1287 (87), PNAS. |
| Val-Tyr-Lys (RIP) | | 5476 (80), Tetrahedron |
| | | 661 (88) |
| His-Pro-Phe-His-Leu-D-Leu-Val-Tyr-OH | — | Biochemistry 3877 (73) |
| Pro-His-Pro-Phe-His- | 10 | JMC 671 (88), Biochem |
| Leu(CH₂NH)Val-Ile-His-Lys (H-142) | | Soc Trans1029(85); Szelke |
| | | review |
| Boc-Phe-His-Cha-(CH₂NH)Val- | 86 | BBRC 982 (86) |
| NH-2(S)-Me(Bu) | | |
| Pro-His-Pro-Phe-His-Leu-Phe---Val-Tyr-OH | — | Biochemistry 3892 (75) |
| Boc-His-Pro-Phe-His- | 0.7 | Szelke review |
| Leu(CH(OH)CH₂)Val-Ile-His-OH | | |
| (H-261) | | |
| PEC-Phe-His-ACHPA- | <0.01 | J. Hypertens.S23 (87) |
| ILeNHC(CH₂OH)₂CH₃ | | |

AMP = 2-aminomethylpyridine
AMB = 3-aminomethylbenzylamine

The Linkers L1 and L2:

A variety of different linkers or linking groups L1 and L2 may be used to link the blood component with the renin inhibitor. The linking groups may be divalent or polyvalent. For example, in the complex of Formula I, L1 may be n-valent where it is attached to Pr, and m-valent where it attaches to Ih where m and n are integers as defined above. Similarly, in the complex of Formula II, L2 may be o-valent where it is attached to Pr and p-valent where it is attached to Ih, where o and p are as defined above. Non-exclusive examples of functional groups that may be present in a linking group include compounds that have a hydroxyl groups, such as N-hydroxysucciimmde, N-hydroxysulfosuccinimide, and other compounds such as maleimide-benzoyl-succinimide, emaleimido-butyryloxy succinimide ester, maleimidopropionic acid, N-hydroxysuccinimide, isocyanate, thioester, thionocarboxylic acid ester, imino ester, carbodiimide, anhydride, or ester.

In addition, certain linng groups having functional groups such as carboxylate, acid halide, azido, diazo, carbodiimide, anhydride, hydrazine, aldehydes, thiols, or amino group may be used to form amides, esters, imines, thioethers, disulfides, substituted amines, or the like. Other specific examples of functional groups that may be employed include acyloxymethylketones, aziridines, diazomethyl ketones, epoxides, iodo-, bromo- or chloroacetamides, olhaloesters, oe haloketones, sulfoniums, chloroethylsulfides, O-alkylisoureas, alkyl halides, vinylsulfones, acrylamides, vinylpyridines, organometallic compounds, aryldisulfides, thiosulfonates, aldehydes, nitrites, α-diketones, α-ketoamides, α-ketoesters, diaminoketones, semicarbazones, and dihydrazides.

The nature and type of compounds that may be selected as the linker depends on the type of reactions, the relative reactivities, selectivities, reversibility and stability characteristics that are desired among the renin inhibitors, the linker and the functional groups on albumin or the blood component. For example, certain reactions that form the conjugate complex arise from an alkylation reaction, a Michael type reaction, an addition-elimination reaction, an addition to sulfur, carbonyl, or cyano groups, or the formation of a metal bond.

Typically, the covalent bond that is formed from these reactions are stable during the active lifetime of the renin inhibitor. In one embodiment, the covalent bond that is formed in these complexes remain stable unless the biologically active subunit is intended to be released at the active site.

The linkers may comprise of compounds having bifunctional or polyfunctional groups that are available for linking a protein such as albumin to multiple renin inhibitors or for linking multiple albumins to a single renin inhibitor.

In a particular preferred embodiment, the linker comprises polyfunctional groups that link a HSA to one or more renin inhibitors. In one embodiment, lining compounds as used herein include any compounds that can link the renin inhibitor to the protein in a single step. In another embodiment, the linking compounds are linked to the renin inhibitor first to form a inhibitor-linker intermediate that can be further reacted with the protein. In another embodiment, the linking compounds are reacted with the protein first to form a protein-linker intermediate that can be further reacted with the renin inhibitor. In each of the above permutations, optionally, the linked compounds may be further purified and/or isolated before submitting to further reactions to form the complex of Formula I or Formula II.

Non-exclusive examples of such polyfunctional compounds include compounds having at least one functional group selected from the group consisting of azidobenzoyl hydrazide, N-[4-(p-azidosalicylamino)butyl]-3′-[2′-pyridyldithio)propionamide), bis-sulfosuccinimidyl suberate, dimethyl adipimidate, disuccinimidyl tartrate, N-y-maleimidobutyryloxysuccinimide ester, N-hydroxy sulfosuccinimidyl-4-azidobenzoate, N-succinimidyl[4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate.

Any linker or linking group that is convenient for use and subject to standard chemical transformations, or linkers that form compounds that are physiologically acceptable at the desired dosages, and are stable in the bloodstream for the desired period of time, may be employed. The linking group may be aliphatic, alicyclic, aromatic, heterocyclic, or combinations thereof. Examples of groups that may be employed as a linking group include alkylenes, arylenes, aralkylenes, cycloalkylenes, polyethers and the like. In a particular embodiment, polyfunctional polyethylene glycol (PEG) and their derivatives may also be employed as linkers.

The linking groups may have at least one atom in the linking chain, more preferably between 1 and 200 atoms in the chain, most preferably between 2 and 50 atoms in the chain. The atoms in the chain can be linear or the chain can be part of one or more rings, each substituted or unsubstituted, and the chain may include carbons or heteroatoms selected from the group consisting of O N, P and S. The rings may be aliphatic, heterocylic, aromatic or heteroaromatic or mixtures thereof, each substituted or unsubstituted. In some embodiments, amino acids or peptides or amino acids employed with mixtures of the above may be used as a linking group.

In one embodiment, L1 is absent and Ih is attached directly to Pr. In another embodiment, L2 is absent and Ih is attached directly to Pr.

In another embodiment for the complex of Formula I, L1 is a linking group that is capable of linking more than one Ih to one Pr, for example, where m is 2 or is more. In one embodiment, m is 1, 2 or 3 and n is 1-30. In one preferred embodiment for the complex of Formula I, Pr is albumin and n is 1. In another particular embodiment, Pr is albumin, Ih is a renin inhibitor, and n is 2-25.

In another embodiment for the complex of Formula II, L2 is a linking group that is capable of linking more than one Pr to one Ih, for example, in the case where o is 2 or more. In one embodiment, Pr is albumin, Ih is a renin inhibitor, o is 1, 2 or 3 and p is 1-5.

In another embodiment, the linking group may be absent in cases where the inhibitor, such as a renin inlnbitor, can be reacted directly with a protein, optionally using a catalyst or coupling agent, such that the complex that is formed comprises only of the renin inhibitor that is directly attached to the protein. An example of such a direct coupling reaction is a mixed anhydride activated coupling reaction of a carboxylic acid followed by the coupling reaction of the intermediate mixed anhydride.

The Protein Component Pr:

Various blood components may be used to prepare the isolated complexes of the present invention. Naturally occurring blood components include blood proteins, which include red blood cells, and immunoglobulins, such as IgM and IgG, serum albumin, transferrin, p90 and p38. In a preferred embodiment, the blood component or blood protein is albumin. More preferably, the albumin is a protein human serum albumin (HSA).

The albumin used in the present invention may also be recombinant albumin. For example, the recombinant human albumin may be produced by transforming a microorganism with a nucleotide coding sequence encoding the amino acid sequence of human serum albumin.

Generally, there exists a very broad range of different methods available for the isolation of compounds from blood or blood plasma that provide a very broad range of final purities, and yields of the product. Albumin is the main protein present in blood plasma, and may be extracted from blood, for examples as disclosed in JP 03/258 728, EP 428 758, EP 452 753, and U.S. Pat. No. 6,638,740 and references cited therein. Further examples of non-exclusive methods for the isolation of various compounds may be based on selective reversible precipitation, ion exchange chromatography, protein affinity chromatography, hydrophobic chromatography, thiophilic chromatography (J. Porath et al; FEBS Letters, vol. 185, p.306, 1985; K. L. Knudsen et al, Analytical Biochemistry, vol 201, p.170, 1992), and various resin matrices (WO 96/00735; WO 96/09116). Certain blood components of established purity are commercially available.

Preparation of Linked Compounds Ih-L1 and Ih-L2:

In one embodiment, the linked compounds Ih-L1 or Ih-L2 of the present invention may be prepared and used in the conjugation with albumin without further purification and/or isolation. The purity of the linked compounds will depend on the nature of the linker, the nature of Ih, and the type of reaction and reaction conditions employed to attach Ih to the linker. In another particular embodiment, the unpurified linked compounds are prepared and obtained with a purity of at least 90%, preferably at least 95%, more preferably at least 97%, and most preferably at least 98%.

In a particular embodiment, the present invention relates to methods for the preparation of the isolated linked compounds, that is, Ih-L1 or Ih-L2. In a preferred embodiment, the isolated linked compounds Ih-L1 and IhL2 are renin inlubitors that are attached to a linker. In one embodiment, the isolated linked compounds may be purified before conjugating with Pr. In another particular embodiment, the linked compounds Ih-L1 or Ih-L2 are isolated and purified to a purity of at least 95%, preferably at least 97%, more preferably at least 98%, and most preferably at least 99% or more.

The linked compounds may be prepared using standard methods known in the art of chemical synthesis. The compounds may be purified using standard methods known in the art, such as by column chromatography or HPLC to provide purified products suitable for in vivo applications. The linked compounds may be frther conjugated with a protein, such as albumin to form the complex of Formulae I and II.

Preparation of Linked Compounds Pr-L1 and Pr-L2:

For certain applications of the present invention, the compounds as represented by Pr may be albumin, may be used as obtained from commercial sources without further purification or isolation, to prepare the linked compounds Pr-L1 and Pr-L2. In a particular embodiment, Pr is HSA. In another embodiment, the albumin may be further purified using various methods known in the art as disclosed herein.

In one embodiment, the linked compounds Pr-L1 and Pr-L2 may be prepared by treating a linker L1 or L2, which may be derivatized or activated, with Pr, in a solution and monitoring the reaction mixture until the reaction is substantially complete. In a particular preferred embodiment, Pr is a protein. In another preferred embodiment, the protein is HSA or recombinant HSA.

In another preferred embodiment, the linked compounds Pr-L1 or Pr-L2 obtained are substantially pure; that is, the linked compounds are obtained with a purity of at least 10%, preferably at least 30%, and more preferably at least 50%. Where the Pr is HSA or recombinant HSA, components that may be present with the linked compounds may comprise of unreacted HSA and various biological components that are present in the HSA starting material. Preferably, the HSA or recombinant HSA is at least 10% pure on a dry matter basis.

An excess of HSA or HSA related biologically materials present with the linked compounds will not significantly interfere with the subsequent conjugation step with Ih. In addition, the related biological materials and the conjugated complexes will also be pharmacologically safe for use in vivo.

However, in certain embodiments, the purity of the linked compounds Pr-L1 or Pr-L2 may be at least 10% on a dry matter basis to enable the selective reaction of the compounds with Ih without a significant amount of interferences or without the formation of undesirable by-products obtained from the conjugate reaction with other undesired blood components. However, the desired purity of Pr, such as HSA or recombinant HSA, for example, will depend on the nature of the functional groups on Ih as well as the functional groups employed on the linker. Typically, higher purities of HSA or recombinant HSA is required if the functional groups on the linker are more reactive and may form undesired by-products than functional groups on the linker that are less reactive.

The albumin maybe obtained from plasma or blood albumin from a host, purified to a desired level of purity, and linked with the linker. Purification of the albumin from blood or blood plasma may be performed using well established standard methods known in the art for the purification of albumin. Using purified blood albumin, the isolated complexes of the present will comprise of a relatively homogeneous population of functionalized proteins.

Preparation of the Complexes of Formula I or Formula II:

In one embodiment, the complexes of Formula I or Formula II may be prepared by the conjugation of lh-L1 or Ih-L2 with Pr, the conjugation of Pr-L1 or Pr-L2 with Ih, or the conjugation of Ih with Pr to form a complex wherein the linker is absent.

In one embodiment, a solution of Ih-L1 or Ih-L2 is combined with Pr under conditions such that the conjugation reaction is deemed to be complete. In a particular embodiment, the linked compound is a renin inhibitor that is attached to a linker, and the linked compound is added to an aqueous solution of HSA. The resulting solution is incubated until the reaction is substantially complete.

In one embodiment, the Ih-L1 or Ih-L2 is combined with an excess of HSA to ensure that the conjugation reaction proceeds selectively to a single site on the HSA. For example, the formation of Ih-L1 on a single site on HSA may permit ease of identification of a single complex of Formula I, for example, where n is 1. In one particular embodiment, the conjugate reaction of Ih-L1 or Ih-L2 with HSA occurs on a single cysteine of HSA. Without being bound by any particular theory, for some reactions, it is believed that the conjugate reaction may also occur initially with a cysteine —SH group to form a kinetic product that is then rearranged to another amino acid functional group, such as a lysine, to form the thermodynamic product.

In another embodiment, the conjugate reaction may form the complex of Formula I, for example, wherein more than one Ih is linked to a single HSA to form the complex of Formula I; that is, wherein n is greater than 1. Optionally, m may be greater than 1 if the linker L1 is a polyfunctional linker that is capable of attaching more than one Ih group. In one embodiment, the complex of Formula I may be prepared by combining an excess of Pr relative to (Ih)m-L1. Preferably, the ratio of Pr to (Ih)m-L1 is about 50 to 100. In another particular embodiment, the ratio is from about 10 to 30. In yet another particular embodiment, the ratio is from about 2 to 5.

In one embodiment, Pr is added to (Ih)m-L1 in a ratio of at least about 1.1:1, more preferably at least about 1.2:1, and most preferably at least about 1.4:1. In the case where Pr is albumin, the preferred ratios are based on the assumption that there is 0.7 free thiol per albumin. Preferably, the resulting complex is formed as a 1:1 complex, since a Pr component such as albumin has only about 70% free thiol functionality for conjugation. An excess of Pr, such as HSA or recombinant HSA is pharmacologically safe and may not require fither purification. Where there is an excess of Pr in the product mixture, optionally, the conjugated complex may be purified to a purity of at least 10%. In a particular embodiment, the conjugated complex may be purified to at least about 20% or at least about 30%.

In another embodiment, the complex of Formula I may be prepared by combining an excess of (Ih)m-L1 relative to Pr. Preferably, the ratio of (Ih)m-L1 to Pr is about 50 to 100. In another particular embodiment, the ratio is from about 10 to 30. In yet another particular embodiment, the ratio is from about 2 to 5. Where there is an excess of (Ih)m-L1 in the product mixture, optionally, the conjugated complex may be purified to a purity of at least 10%. In a particular embodiment, the conjugated complex maybe purified to at least about 20% or at least about 30%.

In another embodiment, the complexes of Formula I or Formula II maybe prepared from a stoichiometric ratio of (Ih)m-L1 with Pr or a stoichiometric ratio of Ih with L2-(Pr)o, that is, in a 1:1 ratio. Optionally, the resulting product from these preparations may be further purified to a purity of at least 10%. In a particular embodiment, the conjugated complex may be purified to at least about 20% or to a purity of at least about 30%. In yet another particular embodiment, the 1:1 conjugated complex may be further purified to a purity of greater than about 90%.

In another embodiment, the conjugated cysteine present in albumin is reduced to the free cysteine prior to the reaction.

Optionally, the complex formed from the conjugate reaction may be further purified prior to administration.

In one embodiment, the complexes of Formula I or Formula II obtained from the conjugate reaction may be administered without further processing or purification since an excess of HSA or HSA related biologically materials present with the complexes are pharmacologically safe for use in vivo.

In each of the above embodiments, Ih is a peptide or peptidomimetic renin inhibitor and Pr is HSA or recombinant HSA.

In one embodiment, the isolated complex comprising a protected or unprotected renin inhibitor with a linker and albumin may be optionally further purified and then returned to the host.

The complexes formed from the methods of the present invention may be tested in animal or human hosts until the physiology, pharmacokinetics, and safety profiles are well established over an extended period of time. Typically, the measured half-life of the complexes is about 5 to 7 days, more typically at least about 7 to 10 days, and preferably 15 to 20 days or more. In general, the duration is species dependent. For example, with human albumin, the half life is about 17-19 days. Depending on the nature of the renin inhibitor, the linking group and the purity of the albumin, the effective therapeutic concentration of the complexes may be at least 1 month or more.

Half lives may be determined by serial measurements of whole blood, plasma or serum levels of the complexes of Formula I or Formula A, the Ih-L compounds, the L-Pr compounds, or the Ih compounds following labeling of the complex or compounds with an isotope (e.g., 131I, 125I, Tc, Cr, 3H, etc . . . ) or fluorochrome and injection of a known quantity of labeled complex or compound intravascularly. Included are red blood cells (half life ca. 60 days), platelets (half life ca. 4-7 days), endothelial cells lining the blood vasculature, and long lived blood serum proteins, such as albumin, steroid binding proteins, ferritin, α2-macroglobulin, transferrin, thyroxin binding protein, immunoglobulins, especially IgG, etc. In addition to preferred half lives, the subject components are preferably in cell count or concentration sufficient to allow binding of therapeutically useful amounts of the compound of the present invention. For cellular long lived blood is components, cell counts of at least 2,000/μl and serum protein concentrations of at least 1 μg/ml, usually at least about 0.01 mg/ml, more usually at least about 1 mg/ml, are preferred.

However, where the nature of the complex is designed such that the biologically active agent Ih, such as a renin inhibitor, is to be cleaved from the complex and released into the host, the desired half life for the effective therapeutic concentration of the complex and/or the biologically active agent may vary from the measured half-life above. The rate of release of the biologically active agent depends in part, on the valency or the functionality on the biological agent which is to be released, the nature of the linking group, the purity and type of the protein, the composition for administration, the manner of administration, and the like. Ihus, various linking groups and biological agents may be employed, where the environment of the blood, components of the blood, particularly enzymes, activity in the liver, or other agent may result in the cleavage of the linking group with release of the biological agent in the host at a desired rate.

The isolated complexes of the present invention provides biological active compounds that have improved pharmacokinetics, solubility, bioavailability, distribution, and/or immunogenicity characteristics as compared to the non-conjugated compounds.

Surprisingly, the complexes of Formula I and Formula II, when prepared and used according to the methods of the present invention, provides an effective therapeutic concentration for a significantly longer time than the Ih component by itself. In addition, the complexes of the present invention provide improved solubility, distribution, pharmacokinetics, and result in decrease immunogenicity when compared to the administration of the Ib component by itself.

The present inventors surprisingly have found that administration to a subject of a conjugate that is prepared ex vivo from purified components (specifically HSA, linker and a renin inhibitor) produces a remarkably efficient tissue vivo distribution of the conjugate compared to conjugates that are prepared by in vivo preparation of the conjugate by injection of an activated compound that binds in situ to endogenous albumin in the blood stream of the subject. Moreover, the present inventors have found that substantially all of the conjugate remains in circulation for hours or even days following administration, compared to the dramatic losses of compound that are observed when the conjugate is prepared in vivo. This efficiency reduces the number of times that the patient must be subjected to injection of active substance, and also reduces the amount of renin inhibitor that must be given in a single administration.

In the context of the present invention, a therapeutically effective amount of a composition is understood to mean an amount that, when administered to a subject, produces a desired physiological effect to a degree that is effective for treatment of a disease, condition, or syndrome in the patient, or that is effective in alleviating the symptoms disease, condition, or syndrome. In particular, a therapeutically effective amount of an antihypertensive complex or composition is understood to mean an amount that, upon administration to a hypertensive subject, produces a desired reduction in systolic and/or diastolic pressure.

Administration of the Isolated Complexes of Formula I and Formula II:

In one embodiment, the administration of the isolated complex of the present invention may be accomplished using a bolus, but may be introduced slowly over time by transfusion using metered flow, or the like.

The complex of the present invention may be administered in a physiologically acceptable medium, e.g. deionized water, phosphate buffered saline, saline, mannitol, aqueous glucose, alcohol, vegetable oil, or the like. A single injection may be employed although more than one injection may be used, if desired. The complex may be administered by any convenient means, including syringe, trocar, catheter, or the like. The particular manner of administration, will vary depending upon the amount to be administered, whether a single bolus or continuous administration, or the like. The administration may be intravascularly, where the site of introduction is not critical to this invention, preferably at a site where there is rapid blood flow, e.g. intravenously, peripheral or central vein. Other routes may find use where the administration is coupled with slow release techniques or a protective matrix.

Surprisingly, it is noted that the administration of the isolated complexes prepared by the methods of the present invention, for example, from isolated blood protein, such as albumin, results in renin inhibitor conjugate complexes that maintain an effective therapeutic effect in the bloodstream for an extended period of time as compared to a non-conjugated renin inhibitor or as compared to complexes that are not prepared from isolated blood protein such as albumin.

In one embodiment, the present invention provides the compounds in the form of a pharmaceutically acceptable salt.

In another embodiment, the present invention provides the compounds present in a mixture of stereoisomers. In yet another embodiment, the present invention provides the compounds as a single stereoisomer.

In yet another embodiment, the present invention provides pharmaceutical compositions comprising the compound as an active ingredient. In yet another particular variation, the present invention provides pharmaceutical composition wherein the composition is a tablet or a solid for administration as a depot. In another particular variation, the present invention provides the pharmaceutical composition wherein the composition is a liquid formulation adapted for IV or subcutaneous administration. In yet another particular variation, the present invention provides pharmaceutical composition wherein the composition is a liquid formulation adapted for parenteral administration.

It is noted in regard to all of the embodiments, and any further embodiments, variations, or individual compounds described or claimed herein that all such embodiments, variations, and/or individual compounds are intended to encompass all pharmaceutically acceptable salt forms whether in the form of a single stereoisomer or mixture of stereoisomers unless it is specifically specified otherwise. Similarly, when one or more potentially chiral centers are present in any of the embodiments, variations, and/or individual compounds specified or claimed herein, both possible chiral centers are intended to be encompassed unless it is specifically specified otherwise.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al.(1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3rd edition, John Wiley & Sons, Inc. 1999.

Compounds of the present invention may also be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxane, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynarnics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which is utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Indications for Use of Renin Inhibitors

The complexes of Formula I and Formula II of the present invention may also be used as renin inhibitors. Renin is an endopeptidase which plays an important role in the control of blood pressure. The renin angiotension system is a multiregulated proteolytic cascade in which renin cleaves the protein substrate angiotensinogen to give angiotensin I. Angiotensin converting enzyme (ACE) catalyses the removal of the terminal dipeptide from the decapeptide angiotensin I to form angiotensin II which exhibits potent pressor activity. Renin is an aspartyl protease with high substrate specificity and is the first proteolytic step in the renin-angiotensin system which is involved in the control of blood pressure. Renin inhibitors have been shown to lower blood pressure in primates, [J. Hypertension, 1, 399 (1983), J. Hypertension 1 (suppl 2), 189 (1983)] and in man, [Lancet II, 1486 (1983), Trans. Assoc. Am. Physicians, 96, 365 (1983), J. Hypertension, 3, 653 (1985] and thus are potentially useful in the control of hypertension.

Injectables

The present invention is also directed to compositions designed to administer the renin inhibitors of the present invention by parenteral administration, generally characterized by injection, either subcutaneously, intramuscularly or intravenously. Injectables may be prepared in any conventional form, for example as liquid solutions or suspensions, solid forms suitable for solution or suspension in liquid prior to injection, or as emulsions.

Examples of excipients that maybe used in conjunction with injectables according to the present invention include, but are not limited to water, saline, dextrose, glycerol, ethanol, or DMSO. The injectable compositions may also is optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut is oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may used include phenols or cresols, mercurials, benzyl alcohol, chlorobutanol, methyl and propyl p-hydroxybenzoic acid esters, thinerosal, benzalkonium chloride and benzethonium chloride.

Examples of isotonic agents that may be used include sodium chloride and dextrose. Examples of buffers that may be used include phosphate and citrate. Examples of antioxidants that maybe used include sodium bisulfite. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of a renin inhibitor complex in the parenteral formulation may be adjusted so that an injection administers a pharmaceutically effective amount sufficient to produce the desired pharmacological effect. The exact concentration of a renin inhibitor complex and/or dosage to be used will ultimately depend on the age, weight and condition of the patient or animal as is known in the art.

Unit-dose parenteral preparations may be packaged in an ampule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of the renin inhibitor to the treated tissue(s). The renin inhibitor complexes may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The renin inhibitor complexes may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

Suitable formulations for each of these methods of administration may be found in, for example, “Remington: The Science and Practice of Pharmacy”, A. Gennaro, ed., 20th edition, (2000), Lippincott, Williams & Wilkins, Philadelphia, Pa.

### REFERENCES

Various methods for the alkylation of albumin have been reported, for example:

Self-quenched fluorogenic substrates for proteolytic enzymes have been prepared by alkylation of thiol groups in reduced bovine serum albumin with iodoacetamidofluorescein or iodoacetamidoeosin. Anal. Biochem. 176:261-264.

Fluorescent derivative with acrylodan. Biophysical Journal Volume 75 August 1998 1084-1096.

The alkylating reagents iodoacetamide, 4-vinylpyridine, and acrylamide are all successful in improving the sequence coverage for albumin.

Alkylation of Cysteines:

Benzyl chlorides: Saunders; BIJOAK; Biochem.J.; 28; 1934; 1977; Kwon, Yeondae; Zhang, Ruoheng; Bemquerer, Marcelo P.; Tominaga, Mineko; Hojo, Hironobu; Aimoto, Saburo; Chem.Lett.; EN; 5; 1993; 881-884.

Alkyl halide: Foti, Salvatore; Saletti, Rosaria; Marletta, Donata; Org.Mass Spectrom.; EN; 26; 10; 1991; 903-907; Jin, Lixia; Baillie, Thomas A.; Chem.Res.Toxicol.; EN; 10; 3; 1997; 318-327; Franzen, Henry M.; Nagren, Kjell; Grehn, Leif; Langstroem, Bengt; Ragnarsson, Ulf; J.Chem.Soc.Perkin Trans.1; EN; 1988; 497-502.

Bromoacetamide; Ziegler,E. et al.; Z.Naturforsch.B Anorg. Chem. Org. Chem. Biochem. Biophys. Biol.; GE; 25; 1970; 1417-1420.

Aziridines: Hata, Yoshiteru; Watanabe, Masamichi; Tetrahedron; EN; 43; 17; 1987; 3881-3888.

Methacrylate: Kasai, Takanori; Nishitoba, Tsuyoshi; Shiroshita, Yoshinari; Sakamura, Sadao; Agric. Biol. Chem.; EN; 48; 9; 1984; 2271-2278.

Vinyl sulfones: Homer, L.; Lindel, H.; Phosphorus Sulfur; GE; 15; 1983; 1-8.

α-Halo ketones: Silva, Claudius D′; Seddon, Andrew P.; Douglas, Kenneth T.; J. Chem. Soc. Perkin Trans.1; EN; 1981; 3029-3033; Chari, Ravi V. J.; Kozarich, John W.; J. Amer. Chem. Soc.; EN; 105; 24; 1983; 7169-7171.

Haloacetate: Climie, Ian J. G.; Evans, David A.; Tetrahedron; EN; 38; 5; 1982; 697-711.

Unsaturated ketones: Spanton, Stephen G.; Prestwich, Glenn D.; Tetrahedron; EN; 38; 13; 1982; 1921-1930. Biophysical Journal Volume 75 August 1998 1084-1096.

Acrylonitrile: Climie, Ian J. G.; Evans, David A.; Tetrahedron; EN; 38; 5; 1982; 697-711.

Acrylamide: Harrison, M. E.; Baldwin, M. A.; Org. Mass Spectrom.; EN; 24; 1989; 689-693.

β-Chloroketones: Vince, R.; Daluge, S.; J. Med. Chem.; EN; 14; 1971; 35-37.

Epoxide: Jin, Lixia; Baillie, Ihomas A.; Chem. Res. Toxicol.; EN; 10; 3; 1997; 318-327.

Allyl halide: Jin, Lixia; Baillie, Ihomas A.; Chem. Res. Toxicol.; EN; 10; 3; 1997; 318-327.

The entire disclosure of all documents cited throughout this application are incorporated herein by reference.

### RENIN INHIBITOR EXAMPLES

Preparation of Renin Inhibitors Conjugate Complexes

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the formation of mixtures of different stereoisomers (enantiomers, diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this Application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3rd edition, John Wiley & Sons, Inc. 1999.

Compounds according to the present invention may be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taling advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization.

As used herein the symbols and conventions used in these processes, schemes and examples are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials are obtained from commercial suppliers and used without further purification.

Synthetic Schemes for Renin Inhibitors of the Present Invention

Renin inlubitors according to the present invention may be synthesized according to a variety of reaction schemes. Some illustrative schemes are provided herein in the examples. Other reaction schemes could be readily devised by those skilled in the art.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

Compounds according to the present invention may optionally be synthesized according to the following general reaction schemes:

### Preparation of Complex of Formula I:

mIh+L1→(Ih)ₘ-L1
n(Ih)ₘ-L1+Pr→[(Ih)ₘ-L1]ₙ-Pr Formula I

### Preparation of Complex of Formula II:

L2+oPr→L2(Pr)ₒ
pL2-(Pr)ₒ+Ih→Ih -[L2-(Pr)ₒ]ₚ Formula II

### EXAMPLE 13

### Conjugation Reaction to Form the Complex

A 10 mM solution of maleimidopropionylaminoethoxyethoxyacetyl derivatized peptide in DMSO was added to a 25% water solution of HSA. The final peptide concentration in the reaction mixture was 1 mM and the molar ratio in reaction mixture of peptide: HSA was 1:4. The solution was incubated 5 hours at 37° C. Once incubation was complete, the conjugate was stored at 4° C.

### EXAMPLE 14

### Conjugation Reaction to Form the Complex

A 10 mM solution of trans4-(maleimidyhmethyl)cyclohexane-1-carbonyl derivatized peptide in DMSO is added to a 25% water solution of HSA. The final peptide concentration in the reaction mixture is 1 mM and the molar ratio in reaction mixture of peptide: HSA is 1:4. The solution is incubated 5 hours at 37° C. Once incubation was complete, the conjugate is stored at 4° C.

### EXAMPLE 15

### Conjugation Reaction to Form the Complex

A 10 mM solution of N-(3-{2-[2-(3-amino-propoxy)-ethoxy]-ethoxy]-ethoxy}-propyl)-2-bromoacetamide derivatized peptide in DMSO is added to a 25% water solution of HSA. The final peptide concentration in the reaction mixture is 1 mM and the molar ratio in reaction mixture of peptide: HSA is 1:4. The solution is incubated 5 hours at 37° C. Once incubation IS complete, the conjugate is stored at 4° C.

### EXAMPLE 16

### Conjugation Reaction to Form the Complex

A 10 mM solution of maleimidopropionylaminoethoxyethoxyacetyl derivatized peptide in DMSO is added to a 25% water solution of HSA. The final peptide concentration in the reaction mixture is 1 mM and the molar ratio in reaction mixture of peptide: HSA is 1:1. The solution is incubated 5 hours at 37° C. Once incubation is complete, the conjugate is stored at 4° C.

### EXAMPLE 17

### Conjugation Reaction to Form the Complex

A 10 mM solution of maleimidopropionylaminoethoxyethoxyacetyl derivatized peptide in DMSO is added to a 25% water solution of HSA. The final peptide concentration in the reaction mixture is 1 mM and the molar ratio in reaction mixture of peptide: HSA is 10:1. The solution is incubated 5 hours at 37° C. Once incubation is complete, the conjugate is stored at 4° C.

It will be apparent to those skilled in the art that various modifications and variations can be made to the compounds, compositions, kits, and methods of the present invention without departing from the spirit or scope of the invention. Ihus, it is intended that the present invention cover the modifications and variations of this invention provided they come within the scope of the appended claims and their equivalents.

### Examples of In vitro Assays

Various assays to measure renin inhibition activity are descnbed in Cartledge, et al. Ann. Clin. Biochem. 262-278 (2000).

### EXAMPLE 18

### Measurement of Renin Inhibitory Activity In Vitro

Fluorescence Measurement of Renin Inhibitory Activity

One method of measuring renin enzyme activity uses the cleavage of a synthetic peptide substrate in a fluorescence-based microplate reader. The peptide substrate for renin, is linked to a fluorophore (5-(aminoethyl)aminonaphthalene sulfonate, EDANS) at one end and to a nonfluorescent chromophore (4′-dimethylaininoazobenzene-4-carboxylate, DABCYL) at the other. After cleavage by renin, the product (peptide-EDANS) is brightly fluorescent. A 500 μM stock solution of renin substrate can be prepared by adding 877 μL of dimethyl sulfoxide (DMSO) to 1 mg of substrate. This stock solution is added into the assay buffer to a final concentration of 2 μM. A small amount (<3% of the final volume) of renin-containing solution is diluted in the assay buffer. The initial rate of cleavage of fluorogenic substrate is measures by monitoring the increase in fluorescence signal at 490 nm for 5-8 min at 37° C. Our conjugates show activity from subnanomolar to high micromolar in this assay.

1. J Protein Chem 10, 553 (1991)

2. Anal Biochem 210, 351 (1993)

3. Science 247, 954 (1990)

4. J Protein Chem 9, 663 (1990).

Measurement of Plasma Renin Inhibitors

Plasma renin activity is determined based on method originally described by Haber et al. (1). Briefly, plasma samples are divided on two aliquots. One aliquot is incubated for 3-18 h at 37 C, while another aliquot is kept on ice. The angiotensin I concentration is determined using commercial kits in RIA or ELISA format according to manufacturer protocol. The angiotensin I concentration in the aliquot kept at 0-4 C is subtracted from that in 37 C aliquot to give a measure of renin activity.

Activity of renin in plasma can also be measured towards externally added renin peptide substrate using HPLC separation of cleavage products. Cleavage products may be detected by LC-MS analysis. Alternatively, peptide substrate can be modified by fluorophore or chromophore to allow spectrophotometric detection. Plasma proteins can be removed by precipitation prior the HPLC analysis.

Concentration of renin inhibitor and/or renin inhibitor-HSA conjugate in plasma is determined by enzyme-based assay (2), which measures inhibitory potential of plasma sample towards externally added recombinant human renin using commercially available quenched fluorescent substrate (3) at pH 7.0-8.0.

1. Haber E, Koerner T, Page L B, Kliman B, Purnode A. Application of a radioimmunoassay for angiotensin I to the physiologic measurements of plasma renin activity in normal human subjects. J Clin Endocrinol Metab. 1969, 29(10): 1349-55

2. Gulnik S., Erickson, J. W., Yu, B. Protease assay for therapeutic drug monitoring. 2003, WO03040390

3. Wang G T, Chung C C, Holzman T F, Krafft G A. A continuous fluorescence assay of renin activity. Anal Biochem. 1993, 210(2):351-9.

### EXAMPLE 18

### In Vivo Testing

The conjugate is administered intravenously to rats. The inhibitor not conjugated with albumin is administered in a control group. Serum samples are collected at 5 min, 30 min, 1 h, 2 h, 8 h, 24 h, 48 h, and 72 h post dose. Renin inhibitory activity is measured by one of the methods described above. Serum concentrations of peptide or peptide-HSA conjugates were calculated from the calibration curves. Based on results of these experiments the following conclusions maybe drawn:

The control peptide displayed a clearance profile with rapid elimination.

The terminal half-life of HSA conjugates range from 12 to 14 hours, similar to that of HSA in this species.

Antihypertensive activity due to human renin inhibition can be measured in hypertensive rats doubly transgenic for human angiotensinogen with endogenous promoter and human renin with endogenous promoter.

Bohlender, et al. Hypertension 428-434 (1997)

For cases where the inhibition of rat renin is comparable to that of human renin antihypertensive activity can be measured in sodium depleted rats.

Allan, et al. JPET 283:661-665, (1997).

### EXAMPLE 19

### Bio Activity of Renin Inhibitor Derivatives

The information presented above clearly demonstrates that the biotin ring on the Ih-L-Pr complex is accessible for binding to avidin. The next series of experiments is designed to address whether Ih-L-Pr complex, which has an IC50 of about 50 nM in its soluble free acid form, is still bioactive after conjugation to target proteins.

Materials and Methods: The following procedures are done under sterile conditions. Rabbit plasma is obtained from freshly drawn heparinized blood. One 8 mL aliquot of plasma is incubated with 5 micromoles of the Ih-L-Pr complex for 60 minutes at room temperature. Another equal aliquot is similarly incubated with 5 micromoles of the Ih-L-Pr complex. The reaction mixtures are stored at 4° C. overnight. Aliquots of these samples are saved for analysis of total renin inhibitor content by a standard renin radioimmunoassay (RIA). After warming to 37° C., the plasma samples are injected into two autologous rabbits. The rabbits are then bled at defined intervals. The blood is centrifiged for 5 minutes at 2500 rpm and then aliquots of the plasma are analyzed by RIA.

Results: Plasma proteins derivatized with the NHS ester of the Ih-L-Pr complex did indeed maintain the inhibitor in a conformation which remained bioavailable and inhibitory after an extended period of circulation in the blood. Again, the amount of inhibitor detectable has been normalized for the effect of dilution of the plasma by the volume of blood in circulation.

The data shows that the level of free acid of the renin inhibitor Ih falls rapidly and is not detectable after one hour. On the other hand, the modified plasma proteins as the Ih-L-Pr complexes are inhibitory in the renin assay, indicating that the conjugation did not destroy the bioactivity of the inhibitor Ih. Furthermore, the level of the inhibition observed does not significantly decrease until day 10. Several abundant plasma proteins (albumin and immunoglobulins) are long-lived and could account for this delivery profile. These results, therefore, clearly demonstrate that covalent attachment of a derivatized renin inhibitor to plasma proteins, such as albumin, does not destroy the bioactivity of the molecule and significantly increases the lifetime of the inhibitor Ih in the blood.

It is evident from the above results that the subject invention provides for greatly improved treatment involving renin inhibition Ih by the use of the complexes of Formula I and Formula II. By use of the subject invention, the renin inhibitors Ih maintain for extended periods of time, so that repetitive dosages are not required, compliance by the patient is not required, and protection is ensured. The derivatized renin inhibitors of the present invention covalently attach to erythrocytes, plasma proteins and various other vascular components to form the complexes of Formula I and Formula II, while retaining biological activity and are not immunogenic.

While the invention has been described in connection with specific embodiments thereof, it will be understood that it is capable of further modifications, and this application is intended to cover any variations, uses or adaptations of the invention following, in general, the principles of the invention, and including such departures from the present description as come within known or customary practice within the art to which the invention pertains, and as may be applied to the essential features hereinbefore set forth, and as follows in the scope of the appended claims.

1-17. (canceled)

18. An isolated complex of the Formula I or Formula II: 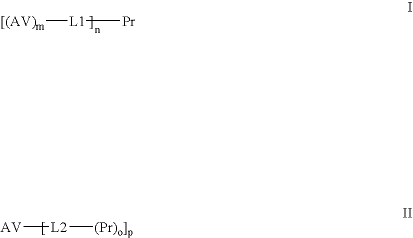
wherein:
m is an integer from 1-5;
n is an integer from 1-100;
o is an integer from 1-5;
p is an integer from 1-100;
AV is an antiviral compound;
L1 and L2 are polyvalent linkers covalently linking AV to Pr, or where L1 and L2 are absent;
Pr is a protein; and
wherein the complex possesses antiviral activity in vivo.

19. The complex of claim 18, wherein the antiviral compound is a peptide.

20. The complex of claim 19 wherein the peptide has a mass of less than about 100 kDA.

21. The complex of claim 19, wherein the peptide has a mass of less than about 30 kDA.

22. The complex of claim 19, wherein the peptide has a mass of less than about 10 kDA.

23. The complex of claim 19 wherein the peptide is a peptidomimetic.

24. The complex of claim 19 wherein the peptide consists of up to 51 amino acids comprising a sequence selected from the group consisting of:
Y1-X-X-Y2-X-X-X-Y3-X-X-X-Y4-X-X-Y5-X-X-Y6;
Y1-X-X-Y2X-X-X-Y3-X-X-X-Y4-X-X-Y5-X-X-Y-X-Y6-Y7;
Y1-X-X-Y2-X-X-X-Y3-X-X-X-Y4X-X-Y5X-X-Y6-Y7-X-X-X-
Y8-X-Y9;
Y1-X-X-Y2-X-X-X-Y3-X-X-X-Y4X-X-Y5X-X-Y6-Y7-X-X-X-
Y8-X-Y9-Y10;
Y1-X-X-Y2-X-X-X-Y3-X-X-X-Y4-X-X-Y5-X-X-Y6-Y7-X-X-
X-Y8-X-Y9-Y10-X-X-Y11;
Y1-X-X-Y2-X-X-X-Y3-X-X-X-Y4-X-X-Y5-X-X-Y6-Y7-X-X-
X-Y8-X-Y9-Y10-X-X-Y11-Y12;
Y1-X-X-Y2-X-X-X-Y3-X-X-X-Y4-X-X-Y5-X-X-Y6-Y7-X-X-
X-Y8-X-Y9-Y10-X-X-Y11-Y12-X-X-Y13;
Y1-X-X-Y2-X-X-X-Y3-X-X-X-Y4-X-X-Y5-X-X-Y6-Y7-X-X-
X-Y-X-Y9-Y10-X-X-Y11-Y12-X-X-Y13-Y14;
Y2-X-X-X-Y3-X-X-X-Y4-X-X-Y5-X-X-Y6-Y7-X-X-X-Y8-X-
Y9-Y10-X-X-Y11-Y12-X-X-Y13-Y14;
Y3-X-X-X-Y4-X-X-Y5-X-X-Y6-Y7-X-X-X-Y8-X-Y9-Y10-X-
X-Y11-Y12-X-X-Y13-Y14;
Y4-X-X-Y5-X-X-Y6-Y7-X-X-X-Y8-X-Y9-Y10-X-X-Y11-Y12-
X-X-Y-13-Y14;
Y5-X-X-Y6-Y7-X-X-X-Y8-X-Y9-Y10-X-X-Y11-Y12-X-X-
Y13-Y14;
Y6-Y7-X-X-X-Y8-X-Y9-Y10-X-X-Y11-Y12-X-X-Y13-Y14;
W-X-X-W-X-X-X-I-X-X-X-T-X-X-I-X-X-L-I-X-X-X-Q-X-Q-
Q-X-X-N;
W-X1-X2-W-X3-X4-X5-I-X6-X7-X8-T-X9-X10-I-X11-X12-
L-I-X13-X14-X15-Q-X16-Q-Q-X17-X18-N-X19-X20-X21-
X22-X23;
peptide DP178 (T-20); and
peptide T-1249;
wherein:
X1 is selected from the group consisting of M, L, I, Q, T, R and K;
X2 is either E, D, Q and K;
X3 is selected from the group consisting of E, D and K;
X4 is selected from the group consisting of K, R, E, Q, N and T;
X5 is selected from the group consisting of E, I, R, K and Q;
X6 is selected from the group consisting of N, D, S, E, Q, K, t, H, T, I and G;
X7 is selected from the group consisting of N, Q, D, E, K, S, T and Y;
X8 is selected from the group consisting of Y, F, H, I, V and S;
X9 is selected from the group consisting of G, K, R, H, D, E, S, T, N and Q;
X10 is selected from the group consisting of K, H, E, Q, T, V, I, L, M, A, Y, F, and P;
X11 is selected from the group consisting of H, K, E, Y and F;
X12 is selected from the group consisting of T, S, Q, N, E, D, R, K, H, W, G, A, and M;
X13 is selected from the group consisting of D, E, Q, T, K, R, A, V and G;
X14 is selected from the group consisting of D, E, K, H, Q, N, S, I, L, V, A and G;
X15 is selected from the group consisting of S, A and (P);
X16 is selected from the group consisting of N, K, S, T, D, E, Y, I and V;
X17 is selected from the group consisting of E, D, N, K, G, and V;
X18 is selected from the group consisting of K, R, H, D, E, N, Q, T, M, I, and Y;
X19 is selected from the group consisting of E, V, Q, M, L, J, and G;
X20 is selected from the group consisting of Q, N, E, K I, H, L, and F;
X21 is selected from the group consisting of E, D, N, S, K, A, and G;
X22 is selected from the group consisting of L, I, and Y; and
X23 is selected from the group consisting of I, L, M, Q, S, and Y.

25. The complex of claim 24 wherein the protein is a blood component.

26. The complex of claim 25, wherein the blood component is selected from the group consisting of red blood cells, immunoglobulins, IgM, IhG, serum albumin, transferrin, P90 and P38, ferritin, a steroid binding protein, thyroxin binding protein, and α-2-macroglobulin.

27. The complex of claim 25, wherein the blood component is human serum albumin and the linker is a peptide linker.

28. The complex of claim 25, wherein the blood component is human serum albumin and the linker is a non-peptide linker.

29. (canceled)

30. The complex of claim 18, wherein the linler L1 or L2 is a non-labile linker that is stable toward hydrolytic cleavage in vivo.

31-47. (canceled)

48. An anti-viral composition comprising a non-peptidic anti-viral compound covalently linked to a blood component.

49-52. (canceled)

53. A method for inhibiting the activity of HIV gp41 and HIV in vivo, the method comprising:
administering to the bloodstream of a mamnmalian host an isolated conjugate complex of claim 18, wherein the complex is formed by attaching an antiviral compound to a linker having at least one reactive functional group which reacts with the protein to form stable covalent bonds; and
wherein the isolated conjugate complex is administered in an amount to maintain an effective therapeutic effect in the bloodstream for an extended period of time as compared to a non-conjugated antiviral compound.

54-80. (canceled)

81. An isolated complex of the Formula I or Formula II:
[(Ih)ₘ-L1]ₙ-Pr I
Ih-[L2-(Pr)ₒ]ₚ II
wherein m is an integer from 1-5; n is an integer from 1-100; o is an integer from 1-5; p is an integer from 1-100; Ih is a renin inhibitor; L1 and L2 are polyvalent linkers covalently linking Ih to Pr, or where L1 and L2 are absent; Pr is a protein; and wherein the complex possesses renin inhibitory activity in vivo.

82-135. (canceled)

136. An isolated compound comprising a pharmacologically active moiety covalently conjugated to a macromolecular carrier,
wherein the carrier is pharmacologically inert,
wherein the linkage between said pharmacologically active moiety and said carrier is stable in vivo,
wherein the intact compound substantially retains the pharmacological activity of said pharmacologically active moiety,
and wherein the active half-life of said compound when administered to a mammal is at least about twice that of said pharmacologically active moiety.

137. The compound according to claim 136, wherein said macromolecular carrier is a protein.

138. The compound according to claim 136, wherein said macromolecular carrier is an albumin of homologous origin to said mammal.

139. The compound according to claim 138, wherein said albumin is a human serum albumin.

140. The compound according to claim 136, wherein said pharmacologically active moiety is conjugated to said carrier via a linker moiety.

141. The compound according to claim 136, wherein said pharmacologically active moiety is directly linked to said carrier.

142. The compound according to claim 136, wherein at least two phannacologically active moiety molecules are conjugated to said carrier.

143. The compound according to claim 137, wherein the linkage to said carrier is via a lysine side chain on said carrier.

144. The compound according to claim 137, wherein the linkage to said carrier is via a cysteine side chain on said carrier,

145. The compound according to claim 136, wherein said carrier is HSA and the linkage is via C34 of the HSA.
